(19) United States
(12) Patent Application Publication
(10) Pub. No.: US 2023/0235353 A1
(43) Pub. Date: Jul. 27, 2023

(54) COMPOSITIONS AND METHODS FOR REDUCING REVERSE PACKAGING OF CAP AND REP SEQUENCES IN RECOMBINANT AAV

(71) Applicant: Ultragenyx Pharmaceutical Inc., Novato, CA (US)

(72) Inventors: Brady Camplin, Quincy, MA (US); Stewart Craig, Cambridge, MA (US); Matthew Scott Fuller, Millis, MA (US); Samuel Wadsworth, Monterey, MA (US)

(21) Appl. No.: 17/911,601

(22) PCT Filed: Mar. 19, 2021
(86) PCT No.: PCT/US2021/023151

§ 371 Date: Sep. 14, 2022

Related U.S. Application Data
(60) Provisional application No. 62/991,768, filed on Mar. 19, 2020.

(51) Int. Cl. C12N 15/86

(52) U.S. Cl. CPC C12N 15/86; C12N 2830/42; C12N 2750/14122; C12N 2750/14143; C12N 2750/14152

(57) ABSTRACT

The present disclosure provides compositions including recombinant nucleic acid constructs, vectors, and host cells, and methods of their use for reducing reverse packaging of cap and/or rep DMA sequences in the production of recombinant adeno-associated vims (rAAV). Also provided are pharmaceutical compositions comprising an rAAV produced from a composition or method of the invention and a pharmaceutically acceptable carrier or excipient. These pharmaceutical compositions may be useful in gene therapy for the prevention or treatment of a disease, condition, or disorder in a subject.

### CROSS-REFERENCE TO RELATED APPLICATIONS

The present application is a U.S. national stage application, filed under 35 U.S.C. § 371, of International Application No. PCT/US2021/023151, filed on Mar. 19, 2021, which claims the benefit of and priority to U.S. Provisional Application No. 62/991,768, filed on Mar. 19, 2020, the entire disclosure of each of which is incorporated herein by reference in its entirety for all purposes.

### SEQUENCE LISTING

The instant application contains a Sequence Listing which has been submitted electronically in ASCII format and is hereby incorporated by reference in its entirety. Said ASCII copy, created on Mar. 15, 2021, is named ULP-008WO_SL.txt and is approximately 156 kilobytes in size.

### TECHNICAL FIELD OF THE INVENTION

The present disclosure relates generally to compositions and methods for producing recombinant adeno-associated virus (rAAV).

### BACKGROUND OF THE INVENTION

Adeno-associated virus (AAV) is a non-pathogenic, replication-defective parvovirus. Recombinant AAV (rAAV) have many unique features that make them attractive as delivery vectors for gene therapy. In particular, rAAV vectors can deliver therapeutic genes to dividing and non-dividing cells, and these genes can persist for extended periods without integrating into the genome of the targeted cell. Given the widespread therapeutic applications of rAAV, there exists an ongoing need for improved methods of rAAV vector production.

A current problem being faced by rAAV manufacturers is a phenomenon in which host cell and plasmid DNA gets inadvertently incorporated into the packaged vector genome of the rAAV. Aberrantly packaged host cell DNA and plasmid DNA are referred to as “co-packaged” DNA and “reverse packaged” DNA, respectively. The presence of these impurities in rAAV preparations can provide a persistent source of antigen capable of being recognized by the immune system, leading to unwanted clearance of transduced cells. Thus, decreasing the incorporation of aberrantly packaged DNA remains a priority in the manufacture of AAV-based therapeutic products.

Previous attempts to reduce reverse packaging of rep and cap plasmid DNA are described in the art. For example, Cao and colleagues describe the creation of intron-modified Ad helper plasmids in which an intron is inserted in the rep gene. See Cao et al., 2000, J. Virology 74(24): 11456-63. However, this strategy was found to significantly reduce genome titers and was ineffective at eliminating the production of undesired replication-competent AAV. Moreover, Cao et al. observed that a relatively high proportion of total packaged particles (˜0.02%) still contained at least some rep DNA sequences. Meanwhile, Halbert et al. successfully decreased reverse packaging of cap DNA by introducing a large intron in the cap gene (termed “captron”), which was found to reduce Cap expression in cells exposed to vectors made with the captron plasmid. See Halbert et al., 2011, Gene Therapy 18(4): 411-7 and U.S. Pat. No. 10,415,056. However, this approach was shown to have no effect on reducing reverse packaged rep DNA and has limited applicability for robust commercial applications since it relies on a four plasmid (quadruple) transfection in which Rep and Cap are expressed from different plasmids, thereby significantly increasing GMP plasmid costs relative to a traditional three plasmid (triple) transfection in which Rep and Cap are expressed from a single plasmid. Moreover, the captron strategy described by Halbert et al. targets an inherently sensitive region harboring native Rep and Cap splice sites, which has the potential to disrupt normal Rep and Cap splicing and the generation of desired protein isoforms.

Given the limitations of the aforementioned strategies, an improved approach is needed that can reduce aberrant packaging of rep and cap DNA, yet still facilitate unaltered Rep and Cap protein expression enabling robust production of rAAV preparations with decreased DNA impurities. The present invention addresses this need via the development of improved nucleic acid constructs (e.g., Rep/Cap expression plasmids) capable of reducing levels of both reverse packaged rep and reverse packaged cap sequences.

### SUMMARY OF THE INVENTION

This invention provides, among other things, compositions and methods of their use for reducing reverse packaging of cap and/or rep DNA sequences in the production of recombinant adeno-associated virus (rAAV).

In one aspect, the present disclosure provides a recombinant nucleic acid construct comprising an AAV Rep coding sequence and an AAV Cap coding sequence, wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb.

In another aspect, the present disclosure provides a vector comprising a recombinant nucleic acid construct, wherein said recombinant nucleic acid construct comprises an AAV Rep coding sequence and an AAV Cap coding sequence, and wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb. In an exemplary embodiment, the vector is a plasmid.

In yet another aspect, the present disclosure provides a host cell comprising a recombinant nucleic acid construct, wherein said recombinant nucleic acid construct comprises an AAV Rep coding sequence and an AAV Cap coding sequence, and wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb. In an exemplary embodiment, the recombinant nucleic acid construct in said host cell is present on a vector, e.g., a plasmid. In some embodiments, the host cell further comprises a plasmid containing one or more adenoviral helper genes (e.g., E1, E2A, E4, VA RNA, etc.) (an “Ad helper” plasmid). In some embodiments, the host cell further comprises a plasmid comprising a 5′-inverted terminal repeat (5′-ITR) sequence, a promoter, a transgene coding sequence, and a 3′-inverted terminal repeat (3′-ITR) sequence (a “Cis” plasmid). In some embodiments, the host cell comprises (a) a plasmid comprising a recombinant nucleic acid construct comprising an AAV Rep coding sequence and an AAV Cap coding sequence, wherein said AAV Cap coding sequence has been modified via the insertion of one or more heterologous excisable intron sequences in the VP3 region of said AAV Cap coding sequence; (b) an Ad helper plasmid, and (c) a Cis plasmid. In certain embodiments, the host cell is selected from a Hek293, HeLa, Cos-7, A549, BHK, Vero, RD, ARPE-19, or MRC-5 cell. In an exemplary embodiment, the host cell is a Hek293 cell.

In yet another aspect, the present disclosure provides a method of producing a preparation of recombinant AAV (rAAV), said method comprising culturing a host cell under suitable conditions that promote the production of rAAV, wherein said host cell comprises a recombinant nucleic acid construct comprising an AAV Rep coding sequence and an AAV Cap coding sequence, and wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb. In an exemplary embodiment, the host cell further comprises an Ad helper plasmid and a Cis plasmid. In an exemplary embodiment, the host cell is a Hek293 cell. In some embodiments, the preparation of rAAV contains reduced levels of cap DNA compared to a corresponding preparation of rAAV produced using an unmodified AAV Cap coding sequence. In some embodiments, the preparation of rAAV contains reduced levels of rep DNA compared to a corresponding preparation of rAAV produced using an unmodified AAV Cap coding sequence. In some embodiments, the preparation of rAAV contains reduced levels of rep DNA and cap DNA compared to a corresponding preparation of rAAV produced using an unmodified AAV Cap coding sequence.

In some embodiments, an rAAV produced using a composition or method described herein comprises a packaged vector genome comprising a coding sequence for a protein transgene. In one embodiment, the coding sequence is a native coding sequence. In another embodiment, the coding sequence is a codon-optimized coding sequence. In some embodiments, coding sequence expresses a protein transgene selected from ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit alpha (PCCA), propionyl-CoA carboxylase subunit beta (PCCB), survival motor neuron (SMN), iduronate-2-sulfatase (IDS), alpha-1-iduronidase (IDUA), tripeptidyl peptidase 1 (TPP1), low-density lipoprotein receptor (LDLR), myotubularin 1, acid alpha-glucosidase (GAA), dystrophia myotonica-protein kinase (DMPK), N-sulfoglucosamine sulfohydrolase (SGSH), fibroblast growth factor-4 (FGF-4), rab escort protein 1 (REP1), carbamoyl synthetase 1 (CPS1), argininosuccinate lyase (ASL), arginase, fumarylacetate hydrolase, alpha-1 antitrypsin, methyl malonyl CoA mutase, a cystic fibrosis transmembrane conductance regulator (CFTR) protein, and a dystrophin gene product (e.g., a minidystrophin or microdystrophin).

In some embodiments, the AAV Cap coding sequence encodes a capsid from an AAV of serotype 8, 9, 1, 2, 3, 4, 5, 6, 7, 10, 11, 12, rh10, hu37 (e.g., AAV8, AAV9, AAV1, AAV2, AAV3, AAV4, AAV5, AAV6, AAV7, AAV10, AAV11, AAV12, AAVrh10, AAVhu37), or an engineered variant thereof. In an exemplary embodiment, the AAV capsid is an AAV serotype 8 (AAV8) capsid, an AAV8 variant capsid, an AAV serotype 9 (AAV9) capsid, an AAV9 variant capsid, or an AAV serotype hu37 (AAVhu37) capsid.

In some embodiments, the recombinant nucleic acid construct further comprises one or more nucleic acid sequences selected from a promoter, an AAV intron, and a coding sequence for a selectable marker.

In some embodiments, a single heterologous excisable intron sequence is inserted into the VP3 region of an AAV Cap coding sequence. In some embodiments, at least two (e.g., two, three, four, or more) heterologous excisable intron sequences are inserted into the VP3 region of an AAV Cap coding sequence.

In some embodiments, the heterologous excisable intron sequence comprises at least one splice donor and at least one splice acceptor site.

In various aspects described herein, the heterologous excisable intron sequence has a length of at least 1 kb. In some embodiments, the heterologous excisable intron sequence has a length of at least 1.5 kb. In some embodiments, the heterologous excisable intron sequence has a length of at least 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, or at least 4.0 kb.

In some embodiments, the heterologous excisable intron has a length of 1.0 kb to 5.0 kb. In some embodiments, the heterologous excisable intron has a length of 1.5 kb to 4.5 kb. In some embodiments, the heterologous excisable intron has a length of 1.8 kb to 4.0 kb. In some embodiments, the heterologous excisable intron has a length of 2.0 kb to 3.5 kb.

In some embodiments, the heterologous excisable intron has a length of about 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, or about 4.5 kb.

In some embodiments, the total combined size of the one or more (e.g., one, two, three, four, or more) heterologous excisable introns is about 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, or about 4.5 kb.

In some embodiments, a heterologous excisable intron for use in the present invention is an intron sequence from a gene encoding a protein selected from eukaryotic translation initiation factor 2, subunit 1 (EIF2S1); collagen type I alpha 2 chain (COL1A2); secreted protein acidic and rich in cysteine (SPARC); signal transducer and activator of transcription 3 (STATS); enolase 1 (ENO1); pyruvate kinase (PKM); aldolase, fructose-bisphosphate A (ALDOA); Y-box binding protein 1 (YBX1); guanine nucleotide binding protein {G protein}, beta polypeptide 2-like 1 (GNB2L1); ribosomal protein S3 (RPS3); GNAS complex locus (GNAS); filamin A (FLNA), transferrin receptor (TFRC); polyA binding protein cytoplasmic 1 (PABPC1); ubiquitin like modifier activating enzyme 1 (UBA1); calnexin (CANX); and lactate dehydrogenase A (LDHA).

In some embodiments, the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the heterologous excisable intron is inserted at a location of cap VP3 having a exon splice donor/exon splice acceptor sequence of CAG/G (SEQ ID NO: 32).

In some embodiments, the heterologous excisable intron is inserted at a location of cap VP3 having a exon splice donor/exon splice acceptor sequence of CAG/CTG (SEQ ID NO: 33).

In some embodiments, the heterologous excisable intron is inserted at a location of cap VP3 having a exon splice donor/exon splice acceptor sequence of CAG/GTG (SEQ ID NO: 34).

In some embodiments, provided are recombinant nucleic acid constructs comprising an AAV Rep coding sequence and an AAV Cap coding sequence, wherein said AAV Cap coding sequence encodes a capsid protein of serotype AAV8 and has been modified via the insertion of one or more heterologous sequences in the VP3 region of said AAV Cap coding sequence, and wherein the total length of the one or more heterologous sequences together is at least 1 kb, and wherein the one or more heterologous sequences are selected from the group consisting of SEQ ID NO: 14 inserted at location C-1, SEQ ID NO: 2 inserted at location C-1, SEQ ID NO: 2 inserted at location A-11, SEQ ID NO: 5 inserted at location C-1, SEQ ID NO: 5 inserted at location A-11, SEQ ID NO: 20 inserted at location C-1, SEQ ID NO: 20 inserted at location A-11, SEQ ID NO: 9 inserted at location C-1, SEQ ID NO: 9 inserted at location A-11, and any combination(s) thereof. In some embodiments, the one or more heterologous sequences are excisable intron sequences.

In some embodiments, the AAV Cap coding sequence before modification comprises a nucleotide sequence that is at least 90%, 91%, 92%, 93%, 94%, 95%, 96%, 97%, 98%, or 99% identical to the nucleotide sequence of SEQ ID NO: 38. In some embodiments, the AAV Cap coding sequence before modification comprises the nucleotide sequence of SEQ ID NO: 38.

In some embodiments, provided are recombinant nucleic acid constructs comprising an AAV Rep coding sequence and an AAV Cap coding sequence, wherein said AAV Cap coding sequence encodes a capsid protein of serotype AAV9 and has been modified via the insertion of one or more heterologous sequences in the VP3 region of said AAV Cap coding sequence, wherein the total length of the one or more heterologous sequences together is at least 1 kb, and wherein the one or more heterologous sequences are selected from the group consisting of SEQ ID NO: 14 inserted at location A-4, SEQ ID NO: 2 inserted at location A-4, SEQ ID NO: 2 inserted at location A-5, SEQ ID NO: 5 inserted at location A-4, SEQ ID NO: 5 inserted at location A-5, SEQ ID NO: 20 inserted at location A-4, SEQ ID NO: 20 inserted at location A-5, SEQ ID NO: 9 inserted at location A-4, SEQ ID NO: 9 inserted at location A-5, and any combination(s) thereof. In some embodiments, the one or more heterologous sequences are excisable intron sequences.

In some embodiments, the AAV Cap coding sequence before modification comprises a nucleotide sequence that is at least 90%, 91%, 92%, 93%, 94%, 95%, 96%, 97%, 98%, or 99% identical to the nucleotide sequence of SEQ ID NO: 42. In some embodiments, the AAV Cap coding sequence before modification comprises the nucleotide sequence of SEQ ID NO: 42.

These and other aspects and features of the invention are described in the following sections of the present disclosure.

### BRIEF DESCRIPTION OF THE DRAWINGS

The invention can be more completely understood with reference to the following drawings.

FIG. 1 is an illustration of AAV8 Cap coding sequence (SEQ ID NO: 38). The underlined nucleotides represent the VP3 coding sequence. Fourteen exon splice donor/exon splice acceptor sequences of CAG/G (SEQ ID NO: 32) (bolded) are present in AAV8 Cap coding sequence; of these fourteen (designated A-1 to A-14 from 5′ to 3′), ten are located in the VP3 coding sequence. Two exon splice donor/exon splice acceptor sequences of CAG/CTG (SEQ ID NO: 33) (bolded, italicized, and boxed) are present in AAV8 Cap coding sequence; of these two (designated B-1 to B-2 from 5′ to 3′), one is located in the VP3 coding sequence. One exon splice donor/exon splice acceptor sequence of CAG/GTG (SEQ ID NO: 34) (designated C-1) is located in the VP3 coding sequence and (bolded, italicized, boxed, and shaded). The final three nucleotides (TAA) correspond to the Cap coding sequence stop codon.

FIG. 2 is an illustration showing a portion of the AAV8 Cap VP3 coding sequence (SEQ ID NO: 43) and the exon splice donor/exon splice acceptor sequences at insertion locations designated A-5, A-6, C-1, B-2, and A-7. FIG. 2 also depicts the translated amino acid sequence (SEQ ID NO: 44) that corresponds to the AAV8 Cap VP3 coding sequence.

FIG. 3 is an illustration showing the AAV8 Cap VP3 coding region and locations of the exon splice donor/exon splice acceptor sequences corresponding to the A-5, A-6, C-1, B-2, A-7, A-8, A-9, A-10, A-11, A-12, A-13, and A-14 insertion sites.

FIG. 4 is a bar graph showing the levels of AAV8 cap DNA as measured by qPCR in rAAV8-hFIX products made with a standard Trans plasmid (positive control, pos. ctrl.), with a standard Trans plasmid without Ad helper plasmid (positive control without Ad helper, pos. ctrl. no Ad), without a standard Trans plasmid (negative control, neg. ctrl.), or with a packtron Trans plasmid modified via the insertion of a heterologous intron in the AAV8 Cap VP3 coding sequence (ALDO A-C1, COL1A2-C1, COL1A2-A11, SPARC-C1, SPARC-A11, GNAS2-C1, GNAS-A11, ENO1-C1, or ENO1-A11). 2-fold to 8-fold reductions in AAV8 cap DNA were observed across the majority of nine rAAV8-hFIX products made with a packtron Trans plasmid.

FIG. 5 is a bar graph showing the levels of AAV8 rep DNA as measured by qPCR in rAAV8-hFIX products made with either a standard Trans plasmid (positive control, pos. ctrl.), a standard Trans plasmid without Ad helper plasmid (positive control without Ad helper, pos. ctrl. no Ad), without a standard Trans plasmid (negative control, neg. ctrl.), or with a packtron Trans plasmid modified via the insertion of a heterologous intron in the AAV8 Cap VP3 coding sequence (ALDOA-C1, COL1A2-C1, COL1A2-A11, SPARC-C1, SPARC-A11, GNAS2-C1, GNAS-A11, ENO1-C1, or ENO1-A11). 2-fold to 8.5-fold reductions in AAV8 rep DNA were observed across the majority of nine rAAV8-hFIX products made with a packtron Trans plasmid.

FIG. 6 is a bar graph showing the levels of packaged genome titer as measured by qPCR in rAAV8-hFIX products made with a standard Trans plasmid (positive control, pos. ctrl.), with a standard Trans plasmid without Ad helper plasmid (positive control without Ad helper, pos. ctrl. no Ad), without a standard Trans plasmid (negative control, neg. ctrl.), or with a packtron Trans plasmid modified via the insertion of a heterologous intron in the AAV8 Cap VP3 coding sequence (ALDOA-C1, COL1A2-C1, COL1A2-A11, SPARC-C1, SPARC-A11, GNAS2-C1, GNAS-A11, ENO1-C1, or ENO1-A11). 1.5-fold to 5-fold increases in packaged hFIX vector genome DNA were observed across the majority of nine rAAV8-hFIX products made with a packtron Trans plasmid.

FIG. 7 shows a Western blot examining the ratio of intracellular AAV8 capsid protein expression of VP1, VP2, and VP3 in rAAV8-hFIX products made with either a standard Trans plasmid (positive control, pos. ctrl.), a standard Trans plasmid without Ad helper plasmid (positive control without Ad helper, pos. ctrl. no Ad), without a standard Trans plasmid (negative control, neg. ctrl.), or with a packtron Trans plasmid modified via the insertion of a heterologous intron in the AAV8 Cap VP3 coding sequence (ALDOA-C1, COL1A2-C1, COL1A2-A11, SPARC-C1, SPARC-A11, GNAS2-C1, GNAS-A11, ENO1-C1, or ENO1-A11). The blot indicates that insertion of a heterologous intron into the AAV8 VP3 ORF does not significantly alter the expression level or splicing of the isoforms of the AAV8 Cap proteins.

FIG. 8 is a bar graph showing the levels of AAV8 cap DNA as measured by qPCR in rAAV8 products expressing one of three transgenes—hFIX, eGFP, or mCherry—made with either a standard Trans plasmid (control) or made with a packtron Trans plasmid modified via the insertion of a heterologous intron in the AAV8 Cap VP3 coding sequence (SPARC-A11 or GNAS2-C1). Reductions in AAV8 cap DNA were observed across all six rAAV8 products made with a packtron Trans plasmid in comparison to corresponding rAAV8 products made with a standard Trans plasmid.

FIG. 9 is a bar graph showing the levels of AAV8 rep DNA as measured by qPCR in rAAV8 products expressing one of three transgenes—hFIX, eGFP, or mCherry—made with either a standard Trans plasmid (control) or made with a packtron Trans plasmid modified via the insertion of a heterologous intron in the AAV8 Cap VP3 coding sequence (SPARC-A11 or GNAS2-C1). Reductions in AAV8 rep DNA were observed across all six rAAV8 products made with a packtron Trans plasmid in comparison to corresponding rAAV8 products made with a standard Trans plasmid.

FIG. 10 is a bar graph showing the levels of packaged genome titer as measured by qPCR in rAAV8 products expressing one of three transgenes—hFIX, eGFP, or mCherry—made with either a standard Trans plasmid (control) or made with a packtron Trans plasmid modified via the insertion of a heterologous intron in the AAV8 Cap VP3 coding sequence (SPARC-A11 or GNAS2-C1). Increases in packaged vector genome DNA were observed in rAAV8 products made with a packtron Trans plasmid comprising in the insertion of a GNAS intron in comparison to corresponding rAAV8 products made with a standard Trans plasmid.

FIG. 11 is a bar graph showing the levels of AAV9 cap DNA as measured by qPCR in rAAV9-hFIX products made with either a standard Trans plasmid (positive control, pos. ctrl.), without a standard Trans plasmid (negative control, neg. ctrl.), or with a packtron Trans plasmid modified via the insertion of a heterologous intron in the AAV9 Cap VP3 coding sequence (ALDOA-A4, COL1A2-A4, COL1A2-A5, SPARC-A4, SPARC-A5, GNAS2-A4, GNAS-A5, ENO1-A4, or ENO1-A5). Reductions in AAV9 cap DNA were observed in all rAAV9-hFIX products made with a packtron Trans plasmid relative to a rAAV9-hFIX product made with a standard Trans plasmid.

FIG. 12 is a bar graph showing the levels of AAV9 rep DNA as measured by qPCR in rAAV9-hFIX products made with either a standard Trans plasmid (positive control, pos. ctrl.), without a standard Trans plasmid (negative control, neg. ctrl.), or with a packtron Trans plasmid modified via the insertion of a heterologous intron in the AAV9 Cap VP3 coding sequence (ALDOA-A4, COL1A2-A4, COL1A2-A5, SPARC-A4, SPARC-A5, GNAS2-A4, GNAS-A5, ENO1-A4, or ENO1-A5). Reductions in AAV9 rep DNA were observed in all rAAV9-hFIX products made with a packtron Trans plasmid relative to a rAAV9-hFIX product made with a standard Trans plasmid.

FIG. 13 is a bar graph showing the levels of packaged genome titer as measured by qPCR in rAAV9-hFIX products made with either a standard Trans plasmid (positive control, pos. ctrl.), without a standard Trans plasmid (negative control, neg. ctrl.), or with a packtron Trans plasmid modified via the insertion of a heterologous intron in the AAV9 Cap VP3 coding sequence (ALDOA-A4, COL1A2-A4, COL1A2-A5, SPARC-A4, SPARC-A5, GNAS2-A4, GNAS-A5, ENO1-A4, or ENO1-A5). Increases in packaged hFIX vector genome DNA were observed across all nine rAAV9-hFIX products made with a packtron Trans plasmid.

### DETAILED DESCRIPTION OF THE INVENTION

Given the limitations of the strategies discussed in the Background section, an improved approach is needed that can reduce aberrant packaging of rep and cap DNA, yet still facilitate unaltered Rep and Cap protein expression enabling robust production of rAAV preparations with decreased DNA impurities. The present invention addresses this need via the development of improved nucleic acid constructs (e.g., Rep/Cap expression plasmids) capable of reducing levels of both reverse packaged rep and reverse packaged cap sequences. And surprisingly, concomitant with the significant reduction of reverse packaged rep and cap sequences, the present inventors observed up to 5-fold higher levels of packaged vector genome when utilizing these improved nucleic acid constructs in a method for the production of rAAV.

This invention provides, among other things, compositions and methods of their use for reducing reverse packaging of cap and/or rep DNA sequences in the production of recombinant adeno-associated virus (rAAV).

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 12 recognized serotypes of AAV (AAV1-12).

Administration/Administer: To provide or give a subject an agent, such as a therapeutic agent (e.g., a recombinant AAV), by any effective route. Exemplary routes of administration include, but are not limited to, injection (such as subcutaneous, intramuscular, intradermal, intraperitoneal, intrathecal, intracerebroventricular, or intravenous administration), oral, intraductal, sublingual, rectal, transdermal, intranasal, vaginal and inhalation routes.

Coding Sequence: A “coding sequence” means the nucleotide sequence encoding a polypeptide in vitro or in vivo when operably linked to appropriate regulatory sequences.

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Excisable: The term “excisable” in reference to an intron refers to an intron that can be removed before translation of a messenger RNA.

Heterologous: The term “heterologous” refers to a gene, nucleic acid, intron, etc., that is foreign, i.e., genotypically distinct, to the entity to which it is being compared. In the context of the instant invention, a heterologous intron refers to an intron which is foreign, i.e., genotypically distinct, from the entity (e.g., AAV or elements of an AAV) in which it is being inserted. For additional clarity, the term “heterologous”, when used in reference to various molecules, e.g., polynucleotides, polypeptides, etc., refers to molecules that are not normally or naturally found in and/or produced by a given entity, e.g., AAV, in nature.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential Cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A region of DNA that directs/initiates transcription of a nucleic acid (e.g., a gene). A promoter includes necessary nucleic acid sequences near the start site of transcription. Many promoter sequences are known to the person skilled in the art and even a combination of different promoter sequences in artificial nucleic acid molecules is possible.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” (“rAAV”) refers to an AAV particle in which a recombinant nucleic acid molecule such as a recombinant nucleic acid molecule encoding a protein transgene has been packaged.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970: Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992: and Pearson et al., Meth. Mol. Rio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Selectable marker: A “selectable marker” may be included on a recombinant nucleic acid construct to allow for the selection of a recipient host cell, e.g., bacterial cell, insect cell, mammalian cell, etc., that has been successfully transformed. Selectable markers that can be expressed in the recipient host cell may include, but are not limited to, genes which render the recipient host cell resistant to drugs such as actinomycin C₁, actinomycin D, amphotericin, ampicillin, bleomycin, carbenicillin, chloramphenicol, geneticin, gentamycin, hygromycin B, kanamycin, methotrexate, mitomycin, neomycin, novobiocin, penicillin, puromycin, rifampicin, streptomycin, tetracycline, and erythromycin. Selectable markers may also include biosynthetic genes, such as those in the histidine, tryptophan, and leucine biosynthetic pathways. Upon transfection or transformation of a recipient host cell, the cell is placed into contact with an appropriate selection marker. Selectable markers may also include visual markers, e.g., fluorescent markers such as green fluorescent protein (GFP) or chemiluminescent markers.

Serotype: A group of closely related microorganisms (such as viruses) distinguished by a characteristic set of antigens.

Stuffer sequence: Refers to a sequence of nucleotides contained within a larger nucleic acid molecule (such as a vector) that is typically used to create desired spacing between two nucleic acid features (such as between a promoter and a coding sequence), or to extend a nucleic acid molecule so that it is of a desired length. Stuffer sequences do not contain protein coding information and can be of unknown/synthetic origin and/or unrelated to other nucleic acid sequences within a larger nucleic acid molecule.

Subject: Living multi-cellular vertebrate organisms, a category that includes human and non-human mammals. In some embodiments, the subject is a human. In one embodiment, the human subject is an adult subject, i.e., a human subject greater than 18 years old. In one embodiment, the human subject is a pediatric subject, i.e., a human subject of ages 0-18 years old inclusive.

Synthetic: Produced by artificial means in a laboratory, for example a synthetic nucleic acid can be chemically synthesized in a laboratory.

Untranslated region (UTR): A typical mRNA contains a 5′ untranslated region (5′ UTR) and a 3′ untranslated region (3′ UTR) upstream and downstream, respectively, of the coding region (see Mignone F. et. al., (2002) Genome Biol 3:REVIEWS0004).

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

This invention provides, among other things, a variety of compositions, e.g., recombinant nucleic acid constructs, vectors, and host cells, as well as methods of their use in production of recombinant adeno-associated virus (rAAV). rAAV produced using the compositions and methods described herein can be useful in a variety of therapeutic and diagnostic applications.

### Recombinant Nucleic Acid Constructs

As described in the examples provided herein, the present inventors have created novel recombinant nucleic acid constructs capable of reducing reverse packaging of cap and/or rep DNA sequences in the production of recombinant adeno-associated virus (rAAV). Use of these recombinant nucleic acid constructs enables production of increased purity rAAV particles which, in turn, may reduce the level, or eliminate the need for, an immunosuppressive regimen after transduction in a subject and may allow for longer transgene expression in transduced cells. Concomitant with the significant reduction of reverse packaged rep and cap sequences, the present inventors have observed that utilization of these novel nucleic acid constructs can surprisingly increase genome titers by up to 5-fold when used in methods for the production of rAAV. Thus, significant therapeutic and manufacturing advantages can be derived from the instantly described recombinant nucleic acid constructs.

In accordance with the foregoing, in a first aspect, the present disclosure provides a recombinant nucleic acid construct comprising an AAV replication (“Rep”) coding sequence and an AAV capsid (“Cap”) coding sequence, wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb.

In some embodiments, the AAV Cap coding sequence encodes a naturally-occurring AAV Cap protein (expressed from a naturally-occurring cap gene). In some embodiments, the AAV Cap coding sequence encodes a genetically-engineered variant of a naturally-occurring AAV capsid protein (expressed from a cap gene which has been engineered to express the variant AAV capsid protein).

In various embodiments described herein, the AAV capsid protein expressed from a recombinant nucleic acid construct of the invention can be from an AAV of serotype 1, 2, 3, 4, 5, 6, 7, 8, 9, 10, 11, 12, rh10, hu37 (e.g., AAV1, AAV2, AAV3, AAV4, AAV5, AAV6, AAV7, AAV8, AAV9, AAV10, AAV11, AAV12, AAVrh10, AAVhu37), as well as any one of the more than 100 natural variants isolated from human and nonhuman primate tissues. See, e.g., Choi et al., 2005, Curr Gene Ther. 5: 299-310, 2005 and Gao et al., 2005, Curr Gene Ther. 5: 285-297.

Beyond the aforementioned capsids, also included within the scope of the invention are recombinant nucleic acid constructs capable of expressing non-natural variant AAV capsids which have been engineered to harbor one or more beneficial therapeutic properties (e.g., improved targeting for select tissues, increased ability to evade the immune response, reduced stimulation of neutralizing antibodies, etc.). Non-limiting examples of such engineered variant capsids are described in U.S. Pat. Nos. 9,506,083, 9,585,971, 9,587,282, 9,611,302, 9,725,485, 9,856,539, 9,909,142, 9,920,097, 10,011,640, 10,081,659, 10,179,176, 10,202,657, 10,214,566, 10,214,785, 10,266,845, 10,294,281, 10,301,648, 10,385,320, and 10,392,632 and in PCT Publication Nos. WO/2017/165859, WO/2018/022905, WO/2018/156654, WO/2018/222503, and WO/2018/226602, the disclosures of which are herein incorporated by reference.

In certain exemplary embodiments, the AAV capsid protein expressed from a recombinant nucleic acid construct of the invention is an AAV8 capsid protein.

The AAV8 capsid protein is described in U.S. Pat. Nos. 7,282,199, 7,790,449, 8,318,480, 8,962,330, 8,962,332, 9,493,788, 9,587,250, and 10,266,846. The AAV8 capsid is a self-assembled AAV capsid composed of 3 AAV8 VP proteins:
VP1 (e.g., a protein having the amino acid sequence of SEQ ID NO: 35, 738 amino acids),
VP2 (e.g., a protein having the amino acid sequence of SEQ ID NO: 36, 601 amino acids, corresponding to AAs 138-738 of VP1), and
VP3 (e.g. a protein having the amino acid sequence of SEQ ID NO: 37, 535 amino acids, corresponding to AAs 204-738 of VP1).

For AAV, all three capsid proteins (VP1, VP2, and VP3) are translated from one mRNA. VP3 is the major capsid protein, accounting for approximately 50 of the 60 capsid monomers, while there are approximately 5 copies of each VP1 and VP2 (and thus a ratio of 1:1:10 for VP1:VP2:VP3) per capsid. As used herein, an “AAV8 capsid” refers to an AAV capsid comprising an amino acid sequence having at least 95%, 96%, 97%, 98%, 99% identity to the amino acid sequence of SEQ ID NO: 35. In some embodiments, an AAV8 capsid as described herein comprises an amino acid sequence of SEQ ID NO: 35. In some embodiments, an AAV8 capsid is encoded by the nucleotide sequence of SEQ ID NO: 38. In some embodiments, an AAV8 capsid as described herein is encoded by a sequence that has been modified via the insertion of one or more heterologous excisable intron sequences, and the sequence before the modification is the nucleotide sequence of SEQ ID NO: 38.

In certain exemplary embodiments, the AAV capsid protein expressed from a recombinant nucleic acid construct of the invention is an AAV9 capsid protein.

The AAV9 capsid protein is described in U.S. Pat. Nos. 7,906,111 and 10,265,417. The AAV9 capsid is a self-assembled AAV capsid composed of 3 AAV9 VP proteins:
VP1 (e.g., a protein having an amino acid sequence of SEQ ID NO: 39, 736 amino acids),
VP2 (e.g., a protein having an amino acid sequence of SEQ ID NO: 40, 599 amino acids, corresponding to AAs 138-736 of VP1), and
VP3 (e.g., a protein having an amino acid sequence of SEQ ID NO: 41, 534 amino acids, corresponding to AAs 203-736 of VP1).

As used herein, an “AAV9 capsid” refers to an AAV capsid comprising an amino acid sequence having at least 95%, 96%, 97%, 98%, or 99% identity to the amino acid sequence of SEQ ID NO: 39. In some embodiments, an AAV9 capsid as described herein comprises an amino acid sequence of SEQ ID NO: 39. In some embodiments, an AAV9 capsid as described herein is encoded by the nucleotide sequence of SEQ ID NO: 42. In some embodiments, an AAV9 capsid as described herein is encoded by a sequence that has been modified via the insertion of one or more heterologous excisable intron sequences, and the sequence before the modification is the nucleotide sequence of SEQ ID NO: 42.

As indicated herein, the recombinant nucleic acid construct according to the invention may encode, in some embodiments, an AAV8 capsid or AAV9 capsid. However, in other embodiments, another AAV capsid is selected. Tissue specificity is determined by the capsid type. AAV serotypes which transduce a suitable target (e.g., liver, muscle, lung, or CNS) may be selected as sources for capsids of AAV viral vectors including, e.g., AAV1, AAV2, AAV3, AAV4, AAV5, AAV6, AAV6.2, AAV7, AAV8, AAV9, AAV10, AAV11, AAV12, AAVrh10, AAVrh64R1, AAVrh64R2, AAVrh8, AAVhu37. In addition, AAV yet to be discovered, or a recombinant AAV based thereon, may be used as a source for the AAV capsid. In some embodiments, an AAV capsid for expression by a recombinant nucleic acid construct described herein can be generated by mutagenesis (i.e., by insertions, deletions, or substitutions) of one of the aforementioned AAV capsids or its encoding nucleic acid.

In accordance with this first aspect of the invention, one or more heterologous excisable intron sequences is inserted into the VP3 region of a AAV Cap coding sequence. For example, in the context of AAV8, one or more heterologous excisable intron sequence(s) may be inserted into an AAV8 Cap coding sequence, e.g., a Cap coding sequence having at least 90%, 91%, 92%, 93%, 94%, 95%, 96%, 97%, 98%, or 99% identity to the nucleotide sequence of SEQ ID NO: 38 (before any insertion(s)), or a Cap coding sequence having a nucleotide sequence of SEQ ID NO: 38 (before any insertion(s)). For illustrative purposes, in the context of AAV8, one or more heterologous excisable intron sequence(s) may be inserted in the region spanning nucleotides 610-2214 of the AAV8 Cap coding sequence of SEQ ID NO: 38.

As another example, in the context of AAV9, one or more heterologous excisable intron sequence(s) may be inserted into an AAV9 Cap coding sequence, e.g., a Cap coding sequence having at least 90%, 91%, 92%, 93%, 94%, 95%, 96%, 97%, 98%, or 99% identity to the nucleotide sequence of SEQ ID NO: 42 (before any insertion(s)), or a Cap coding sequence having a nucleotide sequence of SEQ ID NO: 42 (before any insertion(s)). For additional illustration, in the context of AAV9, one of more heterologous excisable intron sequence(s) may be inserted in the region spanning nucleotides 607-2208 of the AAV9 Cap coding sequence of SEQ ID NO: 42.

While the invention is described in connection with the representative AAV8 and AAV9 Cap coding sequences, a skilled artisan equipped with the present disclosure would readily be able to apply the teachings disclosed herein to any AAV capsid.

In some embodiments, the recombinant nucleic acid construct may further comprise one or more promoters. In one embodiment, the promoter is a heterologous promoter, e.g., a LacZ promoter. In one embodiment, the promoter is an AAV promoter, e.g., a P5, P19, and/or P40 promoter. In some embodiments, the recombinant nucleic acid construct may comprise at least one heterologous promoter and at least AAV promoter.

In some embodiments, the recombinant nucleic acid construct may further comprise one or more AAV introns (i.e., a non-heterologous, native AAV intron).

In some embodiments, the recombinant nucleic acid construct may further comprise a coding sequence for a selectable marker. In one embodiment, the selectable marker is a protein conferring drug resistance, for instance, a protein for kanamycin resistance (e.g., KanR), a protein for ampicillin resistance (e.g., AmpR), or a protein for puromycin resistance (e.g., Pac). In another embodiment, the selectable marker is a biosynthetic pathway protein, such as a protein found in the histidine, tryptophan, or leucine biosynthetic pathways. In another embodiment, the selectable marker is a visual marker, e.g., a fluorescent markers such as green fluorescent protein (GFP). In an exemplary embodiment, the recombinant nucleic acid construct comprises a coding sequence for KanR (encoded by kanR), which conveys kanamycin resistance to a transformed recipient host cell.

In accordance with this first aspect of the invention, in some embodiments, one heterologous excisable intron sequence is or more than one heterologous excisable intron sequences are inserted into the VP3 region of the AAV Cap coding sequence (e.g., AAV8 Cap coding sequence, AAV9 Cap coding sequence, or another AAV serotype Cap coding sequence). In some embodiments, a single heterologous excisable intron sequence is inserted into the VP3 region of an AAV Cap coding sequence (e.g., AAV8 Cap coding sequence, AAV9 Cap coding sequence, or another AAV serotype Cap coding sequence). In some embodiments, at least two (e.g., two, three, four, or more) heterologous excisable intron sequences are inserted into the VP3 region of an AAV Cap coding sequence (e.g., AAV8 Cap coding sequence, AAV9 Cap coding sequence, or another AAV serotype Cap coding sequence). In an exemplary embodiment, a single heterologous excisable intron sequence is inserted into the VP3 region of an AAV Cap coding sequence (e.g., AAV8 Cap coding sequence, AAV9 Cap coding sequence, or another AAV serotype Cap coding sequence).

In accordance with this first aspect of the invention, in certain embodiments, the length of the one heterologous excisable intron sequence or the total length of more than one heterologous excisable intron sequences together is at least 1 kb (i.e., at least 1,000 nucleotide bases), which may be inserted into the VP3 region of an AAV Cap coding sequence (e.g., AAV8 Cap coding sequence, AAV9 Cap coding sequence, or another AAV serotype Cap coding sequence). In some embodiments, the length of the one heterologous excisable intron sequence or the total length of more than one heterologous excisable intron sequences together is at least 1.1 kb, 1.2 kb, 1.3 kb, 1.4 kb, 1.5 kb, 1.6 kb, 1.7 kb, 1.8 kb, 1.9 kb, 2.0 kb, 2.1. kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb, or at least 5.0 kb, which may be inserted into the VP3 region of an AAV Cap coding sequence (e.g., AAV8 Cap coding sequence, AAV9 Cap coding sequence, or another AAV serotype Cap coding sequence).

In some embodiments, the length of the one heterologous excisable intron sequence or the total length of more than one heterologous excisable intron sequences together is 1.0 kb to 5.0 kb, which may be inserted into the VP3 region of an AAV Cap coding sequence (e.g., AAV8 Cap coding sequence, AAV9 Cap coding sequence, or another AAV serotype Cap coding sequence). In one embodiment, the length of the one heterologous excisable intron sequence or the total length of more than one heterologous excisable intron sequences together is 1.5 kb to 4.5 kb, which may be inserted into the VP3 region of an AAV Cap coding sequence (e.g., AAV8 Cap coding sequence, AAV9 Cap coding sequence, or another AAV serotype Cap coding sequence). In one embodiment, the length of the one heterologous excisable intron sequence or the total length of more than one heterologous excisable intron sequences together is 2.0 kb to 4.0 kb. In one embodiment, the length of the one heterologous excisable intron sequence or the total length of more than one heterologous excisable intron sequences together is 2.5 kb to 3.5 kb, which may be inserted into the VP3 region of an AAV Cap coding sequence (e.g., AAV8 Cap coding sequence, AAV9 Cap coding sequence, or another AAV serotype Cap coding sequence).

In some embodiments, the length of the one heterologous excisable intron sequence or the total length of more than one heterologous excisable intron sequences together is about 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, or about 4.5 kb, which may be inserted into the VP3 region of an AAV Cap coding sequence (e.g., AAV8 Cap coding sequence, AAV9 Cap coding sequence, or another AAV serotype Cap coding sequence).

In an exemplary embodiment, a single heterologous excisable intron sequence having a length of about 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, or about 4.5 kb is inserted into the VP3 region of an AAV Cap coding sequence (e.g., AAV8 Cap coding sequence, AAV9 Cap coding sequence, or another AAV serotype Cap coding sequence).

Exemplary heterologous excisable intron for use in the present invention may be selected from an intron shown in Table 1 below:

TABLE 1
Heterologous Excisable Introns.

| Intron | Protein Name | ~Kb | SEQ ID NO: |
|---|---|---|---|
| EIF2S1 | Eukaryotic translation initiation factor 2, subunit 1 | 4.3 | 1 |
| COL1A2 | Collagen type 1 alpha 2 chain | 2.6 | 2 |
| COL1A2 | Collagen type 1 alpha 2 chain | 2.9 | 3 |
| SPARC | Secreted protein acidic and rich in cysteine | 2.0 | 4 |
| SPARC | Secreted protein acidic and rich in cysteine | 2.1 | 5 |
| STAT3 | Signal transducer and activator of transcription 3 | 2.1 | 6 |
| STAT3 | Signal transducer and activator of transcription 3 | 3.2 | 7 |
| STAT3 | Signal transducer and activator of transcription 3 | 3.3 | 8 |
| ENO1 | Enolase 1 | 2.4 | 9 |
| ENO1 | Enolase 1 | 2.8 | 10 |
| ENO1 | Enolase 1 | 3.6 | 11 |
| PKM | Pyruvate kinase | 2.3 | 12 |
| PKM | Pyruvate kinase | 3.5 | 13 |
| ALDOA | Aldolase, fructose-bisphosphate A | 2.7 | 14 |
| YBX1 | Y-box binding protein 1 | 2.7 | 15 |
| YBX1 | Y-box binding protein 1 | 3.5 | 16 |
| GNB2L1 | Guanine nucleotide binding protein, β polypeptide 2-like 1 | 1.9 | 17 |
| RPS3 | Ribosomal protein S3 | 2.1 | 18 |
| GNAS | GNAS complex locus | 3.3 | 19 |
| GNAS | GNAS complex locus | 3.7 | 20 |
| FLNA | Filamin A | 2.8 | 21 |
| FLNA | Filamin A | 3.1 | 22 |
| TFRC | Transferrin receptor | 2.3 | 23 |
| PABPC1 | PolyA binding protein cytoplasmic 1 | 2.1 | 24 |
| PABPC1 | PolyA binding protein cytoplasmic 1 | 2.2 | 25 |
| PABPC1 | PolyA binding protein cytoplasmic 1 | 2.3 | 26 |
| PABPC1 | PolyA binding protein cytoplasmic 1 | 2.6 | 27 |
| UBA1 | Ubiquitin like modifier activating enzyme 1 | 2.3 | 28 |
| UBA1 | Ubiquitin like modifier activating enzyme 1 | 3.2 | 29 |
| CANX | Calnexin | 2.2 | 30 |
| LDHA | Lactate dehydrogenase A | 2.2 | 31 |

In some embodiments, the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV1, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%400%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV1, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV2, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%400%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV2, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV3, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%400%, 92%-100%, 93%-100%, 94%-100%, 95%400%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV3, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV4, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%400%, 92%-100%, 93%-100%, 94%-100%, 95%400%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV4, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV5, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV5, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV6, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV6, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV7, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV7, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV8, and the heterologous excisable intron is selected from a sequence which is 80%400%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV8, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV9, and the heterologous excisable intron is selected from a sequence which is 80%400%, 81%-100%, 82%-100%, 83%-100%, 84%-100%, 85%-100%, 86%-100%, 87%-100%, 88%-100%, 89%-100%, 90%-100%, 91%-100%, 92%-100%, 93%400%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV9, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV10, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV10, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV11, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%400%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV11, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAV12, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%400%, 93%-100%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAV12, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAVrh10, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAVrh10, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the AAV Cap coding sequence encodes a capsid protein of serotype AAVhu37, and the heterologous excisable intron is selected from a sequence which is 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%-100%, 96%-100%, 97%-100%, 98%-100%, 99%-100%, 80%-99%, 80%-98%, 80%-97%, 80%-96%, 80%-95%, 80%-94%, 80%-93%, 80%-92%, 80%-91%, 80%-90%, 80%-89%, 80%-88%, 80%-87%, 80%-86%, 80%-85%, 80%-84%, 80%-83%, 80%-82%, or 80%-81% identical to any one of SEQ ID NOs: 1-31. In some embodiments, the AAV Cap coding sequence encodes a capsid protein of serotype AAVhu37, and the heterologous excisable intron is selected from a sequence which is at least 80%, 85%, 90%, 91%, 92%, 93%, 94%, 95%, 96%, 97%, 98%, 99% or more identical to any one of SEQ ID NOs: 1-31. In some embodiments, the heterologous excisable intron comprises a sequence selected from SEQ ID NOs: 1-31. In some embodiments, the heterologous excisable intron consists of a sequence selected from SEQ ID NOs: 1-31.

In some embodiments, the heterologous excisable intron sequence comprises at least one splice donor site. In some embodiments, the heterologous excisable intron sequence comprises at least one splice acceptor site. In some embodiments, the heterologous excisable intron sequence comprises at least one splice donor and at least one splice acceptor site. In an exemplary embodiment, the heterologous excisable intron sequence naturally comprises at least one splice donor and at least one splice acceptor site. In other embodiments, the heterologous excisable intron sequence may be genetically modified to comprise at least one splice donor and/or at least one splice acceptor site.

The heterologous excisable intron may be positioned at any suitable location in the VP3 region of the Cap coding sequence, provided that the intron is placed within the VP3 region at a location that allows for appropriate expression of the VP1, VP2, and VP3 capsid proteins. The recombinant nucleic acid construct may be generated using standard molecular biology techniques and can be assessed for the ability to produce Cap proteins VP1, VP2, and VP3 using Western blot analysis, as described herein.

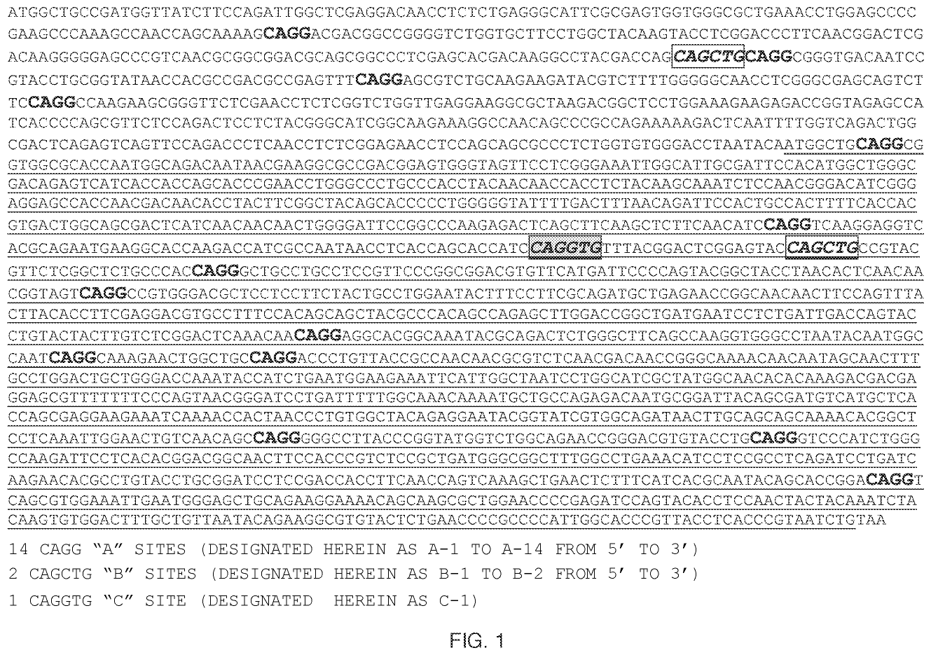

In some embodiments, the heterologous excisable intron is inserted at a location of cap VP3 having a exon splice donor/exon splice acceptor sequence of CAG/G (SEQ ID NO: 32). For purposes of illustration, FIG. 1 shows that 14 such sites exist in the AAV8 Cap coding sequence, of which 10 are located in the VP3 coding sequence.

In some embodiments, the heterologous excisable intron is inserted at a location of cap VP3 having a exon splice donor/exon splice acceptor sequence of CAG/CTG (SEQ ID NO: 33). For purposes of illustration, FIG. 1 shows that two such sites exist in the AAV8 Cap coding sequence, of which one is located in the VP3 coding sequence.

In some embodiments, the heterologous excisable intron is inserted at a location of cap VP3 having a exon splice donor/exon splice acceptor sequence of CAG/GTG (SEQ ID NO: 34). For purposes of illustration, FIG. 1 shows that one such site exists in the AAV8 Cap coding sequence, which is located in the VP3 coding sequence.

In a further embodiment in accordance with this first aspect, the AAV Rep coding sequence may also be modified via the insertion of one or more heterologous excisable intron sequences. Accordingly, in one embodiment, the present disclosure provides a recombinant nucleic acid construct comprising: (a) an AAV Rep coding sequence that has been modified via the insertion of one or more heterologous excisable intron sequences, and (b) an AAV Cap coding sequence that has been modified via the insertion of one or more heterologous excisable intron sequences, wherein said one or more heterologous excisable intron sequences has a total length of at least 1 kb and is/are inserted in the VP3 region of said Cap coding sequence.

### Vectors Comprising a Recombinant Nucleic Acid Construct

In a second aspect, the present disclosure provides a vector comprising a recombinant nucleic acid construct, wherein said recombinant nucleic acid construct comprises an AAV Rep coding sequence and an AAV Cap coding sequence, and wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb. Accordingly, in this aspect, the recombinant nucleic acid construct is included in a genetic element, i.e., a vector, which may be delivered to a host cell.

In some embodiments according to this second aspect, the vector is selected from a plasmid, a cosmid, a phagemid, an episome, a non-viral delivery vehicle (e.g., a lipid nanoparticle), and a virus. In an exemplary embodiment, the vector is a plasmid. In other embodiments, the recombinant nucleic acid construct may be delivered to a host cell as naked DNA.

The selected vector may be delivered to a host cell by any suitable method, including transfection, electroporation, liposome-based delivery, and membrane fusion techniques. In an exemplary embodiment, the vector is delivered to a host cell via transfection. Standard DNA transfection techniques may be used to deliver a vector to a host cell. See, e.g., Sambrook et al., 2000, Molecular Cloning: A Laboratory Manual, 3d. Ed., Cold Spring Harbor Press, Plainview, N.Y.

In some embodiments, the vector, e.g., a plasmid, may further comprise one or more nucleic acid sequences selected from a promoter, an AAV intron, and a coding sequence for a selectable marker.

In an exemplary embodiment, the vector, e.g., a plasmid, may be a “Trans” plasmid having utility in a triple plasmid transfection for the production of rAAV. As used herein, the term “Trans” plasmid refers to a plasmid comprising AAV Rep and Cap genes and from which AAV Rep and Cap proteins are expressed. In some embodiments, the vector is a “packtron Trans plasmid,” which is modified via the insertion of one or more heterologous excisable intron sequences in the VP3 region. (As used herein, the term “standard Trans plasmid” refers to a Trans plasmid which is not modified via the insertion of one or more heterologous excisable intron sequences.)

In some embodiments, the vector, e.g., a plasmid, may further comprise one or more nucleic acid sequences selected from a 5′-inverted terminal repeat (5′-ITR) sequence, and an enhancer sequence, a promoter sequence, an intron sequence, a transgene coding sequence, a polyadenylation signal sequence, a stuffer nucleic acid sequence, and a 3′-inverted terminal repeat (3′-ITR) sequence. In an exemplary embodiment, the vector, e.g., a plasmid, comprises at least a 5′-inverted terminal repeat (5′-ITR) sequence, a promoter sequence, a transgene coding sequence, and a 3′-inverted terminal repeat (3′-ITR) sequence.

Accordingly, in some embodiments, the present disclosure provides a vector comprising (a) a recombinant nucleic acid construct, wherein said recombinant nucleic acid construct comprises an AAV Rep coding sequence and an AAV Cap coding sequence, and wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb; (b) a 5′-inverted terminal repeat (5′-ITR) sequence; (c) a promoter sequence capable of driving transgene expression; (d) a transgene coding sequence; and (e) 3′-inverted terminal repeat (5′-ITR) sequence. In some embodiments, the vector may further comprise at least one sequence element selected from an enhancer sequence, an intron sequence which is different than the intron sequence of part (a), a polyadenylation signal sequence, and a stuffer nucleic acid sequence.

### Host Cells Comprising a Recombinant Nucleic Acid Construct

In a third aspect, the present disclosure provides a host cell comprising a recombinant nucleic acid construct, wherein said recombinant nucleic acid construct comprises an AAV Rep coding sequence and an AAV Cap coding sequence, and wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb. In an exemplary embodiment, the recombinant nucleic acid construct in said host cell is present on a vector, e.g., a plasmid.

In some embodiments, the host cell further comprises a plasmid containing one or more adenoviral helper genes, e.g., adenoviral helper genes such as E1A, E1B, E2A, E4, VA RNA, etc. (referred to herein as an “Ad helper” plasmid). In an exemplary embodiment, the host cell comprises a plasmid which comprises E2A, E4, and VA RNA. In some embodiments, the host cell, such as a Hek293 host cell, is capable of supplying the E1A and E1B function.

In alternative embodiments, helper functions may be supplied by a herpesvirus. In these alternative embodiments, the host cell can further comprise a plasmid containing one or more herpesvirus genes, e.g., herpesvirus replication genes such as ULS, ULB, UL9, UL29, UL30, UL42, and UL52 (referred to herein as an “HSV helper” plasmid).

In some embodiments, the host cell further comprises a plasmid comprising a 5′-inverted terminal repeat (5′-ITR) sequence, a promoter, a transgene coding sequence, and a 3′-inverted terminal repeat (3′-ITR) sequence (referred to herein as a “Cis” plasmid). In some embodiments, the Cis plasmid may further comprise one or more sequence elements selected from an enhancer, an intron, a polyadenylation signal, and a stuffer nucleic acid sequence.

In some embodiments, the Cis plasmid comprises a 5′-ITR sequence from AAV2. In some embodiments, the Cis plasmid comprises a 3′-ITR sequence from AAV2. In some embodiments, the Cis plasmid comprises a 5′-ITR sequence and the 3′-ITR sequence from AAV2. In other embodiments, the Cis plasmid comprises a 5′-ITR sequence and/or a 3′-ITR sequence from a non-AAV2 source.

In some embodiments, the Cis plasmid comprises a promoter selected from a chicken β-actin (CBA) promoter, a cytomegalovirus (CMV) immediate early gene promoter, a transthyretin (TTR) promoter, a thyroxine binding globulin (TB G) promoter, and an alpha-1 anti-trypsin (A1AT) promoter. In some embodiments, the Cis plasmid comprises a promoter which is a gene-specific endogenous promoter.

In addition to a promoter and a coding sequence for at least one transgene, a Cis plasmid may contain other appropriate transcription initiation, termination, and enhancer sequences, and efficient RNA processing signals.

In some embodiments, the Cis plasmid comprises one or more enhancer sequences. In one embodiment, the enhancer is selected from a cytomegalovirus immediate early gene (CMV) enhancer, a transthyretin enhancer (enTTR), a chicken β-actin (CBA) enhancer, an En34 enhancer, and an ApoE enhancer.

In some embodiments, the Cis plasmid comprises one or more intron sequences. In one embodiment, the intron is selected from an SV40 Small T intron, a rabbit hemoglobin subunit beta (rHBB) intron, a human beta globin IVS2 intron, a β-globin/IgG chimeric intron, or an hFIX intron.

In some embodiments, the Cis plasmid comprises a polyadenylation signal sequence. In one embodiment, the polyadenylation signal sequence is selected from a bovine growth hormone (BGH) polyadenylation signal sequence, an SV40 polyadenylation signal sequence, a rabbit beta globin polyadenylation signal sequence.

In some embodiments according to this third aspect, the host cell comprises a Cis plasmid, wherein the Cis plasmid comprises a partial or complete coding sequence for a transgene protein or an isoform thereof, or a functional fragment or functional variant thereof.

In one embodiment, the partial or complete coding sequence for a transgene protein is a wild-type, i.e., “native” coding sequence. As used herein, the term “wild-type” refers to a biopolymer (e.g., a polypeptide sequence or polynucleotide sequence) that is the same as the biopolymer (e.g., polypeptide sequence or polynucleotide sequence) that exists in nature.

In one embodiment, the partial or complete coding sequence for a transgene protein is a codon-optimized coding sequence. In one embodiment, the partial or complete coding sequence is codon-optimized for expression in humans.

In some embodiments, the coding sequence expresses a protein transgene selected from ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit alpha (PCCA), propionyl-CoA carboxylase subunit beta (PCCB), survival motor neuron (SMN), iduronate-2-sulfatase (IDS), alpha-1-iduronidase (IDUA), tripeptidyl peptidase 1 (TPP1), low-density lipoprotein receptor (LDLR), myotubularin 1, acid alpha-glucosidase (GAA), dystrophia myotonica-protein kinase (DMPK), N-sulfoglucosamine sulfohydrolase (SGSH), fibroblast growth factor-4 (FGF-4), rab escort protein 1 (REP1), carbamoyl synthetase 1 (CPS1), argininosuccinate lyase (ASL), arginase, fumarylacetate hydrolase, alpha-1 antitrypsin, methyl malonyl CoA mutase, a cystic fibrosis transmembrane conductance regulator (CFTR) protein, and a dystrophin gene product (e.g., a minidystrophin or microdystrophin).

The invention may be used, for example, to manufacture rAAV capable of delivering these aforementioned protein transgenes, as well as fragments, variants, isoforms, and fusions thereof.

In accordance with this third aspect, provided herein are host cells comprising a recombinant nucleic acid construct, wherein said recombinant nucleic acid construct comprises an AAV Rep coding sequence and an AAV Cap coding sequence, and wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb. In specific embodiments, the host cells may be suitable for the propagation of AAV.

A vast range of host cells can be used, such as bacteria, yeast, insect, mammalian cells, etc. In some embodiments, the host cell can be a cell (or a cell line) appropriate for production of rAAV, for example, a Hek293, HeLa, Cos-7, A549, BHK, Vero, RD, ARPE-19, or MRC-5 cell.

In an exemplary embodiment, the host cell is a Hek293 cell. It will be understood and readily appreciated by the skilled artisan that, included with the meaning of Hek293 cell, is any clonal derivative, e.g., a Hek293-F cell, Hek293-T cell, or a Hek-EXPI293 cell.

In another embodiment, the host cell is a HeLa cell. It will be understood and readily appreciated by the skilled artisan that, included within the meaning of HeLa cell, is any clonal derivative, e.g., a HeLa S3 cell, which is a subclone of the HeLa cell line that can grow in serum-free medium as well as suspension cultures.

The recombinant nucleic acid construct or vector comprising the same can be delivered into the host cell using any suitable method known in the art. In an exemplary embodiment, the recombinant nucleic acid construct or vector comprising the same is delivered via transfection. In some embodiments, a stable host cell line that has the recombinant nucleic acid construct or vector inserted into its genome is generated. In some embodiments, a stable host cell line is generated, which contains a recombinant nucleic acid construct described herein.

In certain exemplary embodiments, a host cell is transfected with a Trans plasmid of the invention, along with an Ad helper plasmid and a Cis plasmid, e.g., a triple transfection suitable for the production of rAAV. In one embodiment, the host cell is a Hek293 cell.

### Methods for Producing Recombinant AAV

In a fourth aspect, the present disclosure provides a method of producing a preparation of recombinant AAV (rAAV), said method comprising culturing a host cell under suitable conditions that promote the production of rAAV, wherein said host cell comprises a recombinant nucleic acid construct comprising an AAV Rep coding sequence and an AAV Cap coding sequence, and wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb. In an exemplary embodiment, the host cell further comprises an Ad helper plasmid and a Cis plasmid. In another exemplary embodiment, the host cell is a Hek293 cell.

In another aspect, the present disclosure provides a method of reducing cap and/or rep DNA contamination in recombinant AAV (rAAV). In some embodiments, the method comprises: (a) introducing into a suitable host cell a vector comprising a recombinant nucleic acid construct as described herein; (b) expressing an Ad helper plasmid and a Cis plasmid in said host cell; and (c) culturing the host cell to produce rAAV.

Methods for producing rAAV suitable for gene therapy are well known in the art (See. e.g., Clement et al., 2016, Mol. Ther. Methods. Clin. Dev. 3: 16002, Naso et al., 2017, BioDrugs 31(4): 317-334, and Ayuso et al., 2010, Curr. Gene Ther. 10(6): 423-36), and can be utilized with the recombinant nucleic acid constructs and methods disclosed herein.

As described above, the selected vector may be delivered to a host cell by any suitable method, including transfection, electroporation, liposome-based delivery, and membrane fusion techniques. In an exemplary embodiment, the vector is delivered to a host cell via transfection.

In some embodiments, the preparation of rAAV contains reduced levels of cap DNA compared to a corresponding preparation of rAAV produced using an unmodified AAV Cap coding sequence. In some embodiments, the preparation of rAAV contains reduced levels of rep DNA compared to a corresponding preparation of rAAV produced using an unmodified AAV Cap coding sequence. In some embodiments, the preparation of rAAV contains reduced levels of cap DNA and rep DNA compared to a corresponding preparation of rAAV produced using an unmodified AAV Cap coding sequence.

In one embodiment, the cap DNA levels may be reduced by at 1.5-fold or more in an rAAV preparation prepared using a modified AAV Cap coding sequence of the present disclosure. In some embodiments, the cap DNA levels may be reduced by at least 1.6-fold, 1.7-fold, 1.8-fold, 1.9-fold, 2-fold, 3-fold, 4-fold, 5-fold, 6-fold, 7-fold, or 8-fold compared to a corresponding preparation of rAAV produced using an unmodified AAV Cap coding sequence.

In one embodiment, the rep DNA levels may be reduced by at 1.5-fold or more in an rAAV preparation prepared using a modified AAV Cap coding sequence of the present disclosure. In some embodiments, the rep DNA levels may be reduced by at least 1.6-fold, 1.7-fold, 1.8-fold, 1.9-fold, 2-fold, 3-fold, 4-fold, 5-fold, 6-fold, 7-fold, or 8-fold compared to a corresponding preparation of rAAV produced using an unmodified AAV Cap coding sequence.

The levels of cap DNA and rep DNA in rAAV preparations can be measured by any suitable method available in the art. See, e.g., Sanmiguel et al., 2019, Adeno-Associated Virus Vectors: Design and Delivery, Methods in Molecular Biology, vol. 1950, Chapter 4, Springer Nature 2019. One such method is quantitative PCR (qPCR), as described in the Examples. Another such method is droplet digital PCR (ddPCR). Other methods include Southern blots, dot blots, slot blots, or any other such methods utilized in the art to detect DNA by hybridization with a labeled probe.

Recombinant AAV (rAAV) and Associated Pharmaceutical Compositions

In an additional aspect, the present disclosure provides an rAAV produced from a host cell comprising a recombinant nucleic acid construct, wherein said recombinant nucleic acid construct comprises an AAV Rep coding sequence and an AAV Cap coding sequence, and wherein said AAV Cap coding sequence has been modified via the insertion of one or more heterologous excisable intron sequences in the VP3 region of said AAV Cap coding sequence, and wherein the total length of the one or more heterologous excisable intron sequences together is at least 1 kb.

In some embodiments according to this aspect, the rAAV has been produced a host cell selected from a Hek293, HeLa, Cos-7, A549, BHK, Vero, RD, ARPE-19, or MRC-5 cell. In an exemplary embodiment, the host cell is a Hek293 cell. In certain exemplary embodiments, the host cell has been transfected with a Trans plasmid of the invention, along with an Ad helper plasmid and a Cis plasmid.

In some embodiments according to the aspect, the rAAV comprises a capsid and a vector genome packaged therein, wherein the vector genome comprises a promoter sequence and a coding sequence for a protein transgene. In some embodiments, the coding sequence expresses a protein transgene selected from ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit alpha (PCCA), propionyl-CoA carboxylase subunit beta (PCCB), survival motor neuron (SMN), iduronate-2-sulfatase (IDS), alpha-1-iduronidase (IDUA), tripeptidyl peptidase 1 (TPP1), low-density lipoprotein receptor (LDLR), myotubularin 1, acid alpha-glucosidase (GAA), dystrophia myotonica-protein kinase (DMPK), N-sulfoglucosamine sulfohydrolase (SGSH), fibroblast growth factor-4 (FGF-4), rab escort protein 1 (REP1), carbamoyl synthetase 1 (CPS1), argininosuccinate lyase (ASL), arginase, fumarylacetate hydrolase, alpha-1 antitrypsin, methyl malonyl CoA mutase, a cystic fibrosis transmembrane conductance regulator (CFTR) protein, and a dystrophin gene product (e.g., a minidystrophin or microdystrophin). In some embodiments, the capsid is from an AAV of serotype 1, 2, 3, 4, 5, 6, 7, 8, 9, 10, 11, 12, rh10, or hu37, or an engineered variant thereof. In an exemplary embodiment, the rAAV comprises an AAV8 capsid. In another exemplary embodiment, the rAAV comprises an AAV9 capsid.

In a further aspect, the present disclosure provides an rAAV of the invention (e.g., an rAAV produced from a host cell or method described herein) and a pharmaceutically acceptable carrier or excipient. In some embodiments, the pharmaceutical composition comprising an rAAV of the invention (e.g., an rAAV produced from a host cell or method described herein) is formulated for intravenous, subcutaneous, intramuscular, intradermal, intraperitoneal, intrathecal, or intracerebroventricular administration.

In some embodiments, the rAAV is formulated in a buffer/carrier suitable for infusion in human subjects. The buffer/carrier should include a component that prevents the rAAV from sticking to the infusion tubing but does not interfere with the rAAV binding activity in vivo. Various suitable solutions may include one or more of: a buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration. The pH may be in the range of 6.5 to 8.5, or 7 to 8.5, or 7.5 to 8. A suitable surfactant, or combination of surfactants, may be selected from among Poloxamers, i.e., nonionic triblock copolymers composed of a central hydrophobic chain of polyoxypropylene 10 (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol.

In an exemplary embodiment, the rAAV is formulated in a solution comprising NaCl (e.g., 200 mM NaCl), MgCl₂ (e.g., 1 mM MgCl₂), Tris (e.g., 20 mM Tris), pH 8.0, and poloxamer 188 (e.g., 0.005% or 0.01% poloxamer 188).

In some embodiments, the rAAV is formulated in a pharmaceutical composition comprising at least one dihydric or polyhydric alcohol. In one embodiment, the dihydric or polyhydric alcohol is one or more alcohols selected from the group consisting of polyethylene glycol, propylene glycol and sorbitol.

In an exemplary embodiment, the rAAV is formulated in a pharmaceutical composition comprising sorbitol. In one embodiment, sorbitol is present in the formulation at a range of 0.5 wt % to 20 wt %. In one embodiment, sorbitol is present in the formulation at a range of 1 wt % to 10 wt %. In one embodiment, sorbitol is present in the formulation at about 1 wt %, 2 wt %, 3 wt %, 4 wt %, 5 wt %, 6 wt %, 7 wt %, 8 wt %, 9 wt %, or about 10 wt %.

In an exemplary embodiment, the rAAV is formulated in a pharmaceutical composition comprising 5 wt % sorbitol and poloxamer 188 (e.g., 0.005% or 0.01% poloxamer 188).

### Methods of Treatment

In yet another aspect, the present disclosure provides methods of preventing, treating or ameliorating a disease, condition, or disorder in a human subject comprising administering to the human subject a therapeutically effective amount of at least one rAAV disclosed herein.

Any suitable method or route can be used to administer an rAAV or an rAAV-containing composition described herein. Routes of administration include, for example, subcutaneously, intradermally, intraperitoneally, intrathecally, intracerebroventricularly, intravenously, and other parenteral routes of administration. In an exemplary embodiment, the rAAV is administered intravenously.

The specific dose administered can be a uniform dose for each patient, for example, 1.0×10¹¹-1.0×10¹⁴ genome copies (GC) of virus per patient. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can also be adjusted as the progress of the disease is monitored

In some embodiments, the rAAV is administered at a dose of, e.g., about 1.0×10¹¹ genome copies per kilogram of patient body weight (GC/kg) to about 1×10¹⁴ GC/kg, about 5×10¹¹ genome copies per kilogram of patient body weight (GC/kg) to about 5×10¹³ GC/kg, or about 1×10¹² to about 1×10¹³ GC/kg, as measured by qPCR or digital droplet PCR (ddPCR). In some embodiments, the rAAV is administered at a dose of about 1×10¹² to about 1×10¹³ genome copies (GC)/kg. In some embodiments, the rAAV is administered at a dose of about 1.1×10¹¹, about 1.3×10¹¹, about 1.6×10¹¹, about 1.9×10¹¹, about 2×10¹¹, about 2.5×10¹¹, about 3.0×10¹¹, about 3.5×10¹¹, about 4.0×10¹¹, about 4.5×10¹¹, about 5.0×10¹¹, about 5.5×10¹¹, about 6.0×10¹¹, about 6.5×10¹¹, about 7.0×10¹¹, about 7.5×10¹¹, about 8.0×10¹¹, about 8.5×10¹¹, about 9.0×10¹¹, about 9.5×10¹¹, about 1.0×10¹², about 1.5×10¹², about 2.0×10¹², about 2.5×10¹², about 3.0×10¹², about 3.5×10¹², about 4.0×10¹², about 4.5×10¹², about 5.0×10¹², about 5.5×10¹², about 6.0×10¹², about 6.5×10¹², about 7.0×10¹², about 7.5×10¹², about 8.0×10¹², about 8.5×10¹², about 9.0×10¹², about 9.5×10¹², about 1.0×10¹³, about 1.5×10¹³, about 2.0×10¹³, about 2.5×10¹³, about 3.0×10¹³, about 3.5×10¹³, about 4.0×10¹³, about 4.5×10¹³, about 5.0×10¹³, about 5.5×10¹³, about 6.0×10¹³, about 6.5×10¹³, about 7.0×10¹³, about 7.5×10¹³, about 8.0×10¹³, about 8.5×10¹³, about 9.0×10¹³, or about 9.5×10¹³ genome copies (GC)/kg. The rAAV can be administered in a single dose, or in multiple doses (such as 2, 3, 4, 5, 6, 7, 8, 9, 10 or more doses) as needed for the desired therapeutic results.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the present disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within the present disclosure, embodiments have been described and depicted in a way that enables a clear and concise disclosure to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including” is intended merely to illustrate better the present invention and does not pose a limitation on the scope of the invention unless claimed. No language in the specification should be construed as indicating any non-claimed element as essential to the practice of the present invention.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1

The purpose of this example is to describe the selection and identification of potential splice donor and acceptor sites in the VP3 ORF of the AAV8 capsid.

In this example, an in silico search for intronic splice donor and splice acceptor DNA motifs within the AAV8 VP3 ORF was completed. Specifically, consensus and very strong splice site motifs were identified containing “CAG/G” (represented by SEQ ID NO: 32), “CAG/CTG” (represented by SEQ ID NO: 33), or “CAG/GTG” (represented by SEQ ID NO: 34) exon splice donor/exon splice acceptor sequences as described previously (See Shapiro & Senapathy, 1987, Nucl. Acids Res. 15(17); Shepard et al., 2011, Nucl. Acids Res. 39(20)).

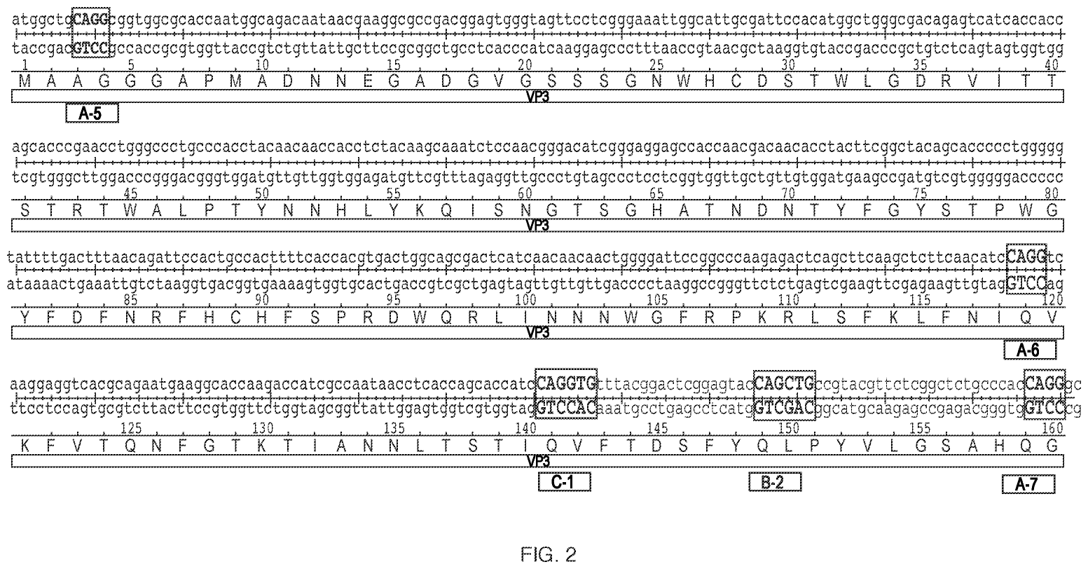

As shown in FIG. 2 for illustrative purposes, three such “CAG/G” motifs, designated A-5, A-6 and A-7, one such “CAG/CTG” motif, designated B-2, and one such “CAG/GTG” motif, designated C-1, were identified within the section of the AAV8 VP3 ORF shown.

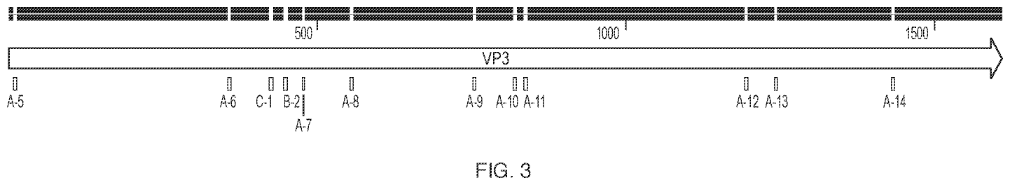

As shown in FIG. 3 for illustrative purposes, ten such “CAG/G” motifs, designated A5-14, one such “CAG/CTG” motif, designated B-2, and one such “CAG/GTG” motif, designated C-1, were identified within the complete AAV8 VP3 ORF.

### Example 2

The purpose of this example is to describe the selection of suitable heterologous excisable introns for insertion into the VP3 ORF of the AAV8 capsid.

In this example, the following criteria were utilized to identify heterologous intron candidates: >1000 base pair length, elevated expression of a gene-specific pre-mRNA or mRNA in a cervical carcinoma cell or elevated expression of a gene-specific pre-mRNA or mRNA in a cervical carcinoma cell infected with Adenovirus serotype 5 (either as described in the literature or as determined empirically by RNA-SEQ analysis), and presence of internal consensus splice acceptor and donor sites, “GT” and “AG”, respectively.

The screening described in the above paragraph led to the selection of the introns shown in SEQ ID NOs: 1-31. Specifically, the EIF2S1 intron (Halbert et al., 2011, Gene Therapy 18(4): 411-7 and Wang et al., 2014, Gene Therapy 21(4): 363-70), the COL1A2 introns (Cao et al., 2000, J. Virology 74(24): 11456-63), the SPARC introns (Shi et al., 2016, Oncol Lett 11(5): 3251-8), and the STATS introns (Shukla et al., 2010, Mol Cancer 9: 282) were described in the literature previously. The remaining introns shown in SEQ ID NOs: 1-31 were identified using RNA-SEQ analysis which was performed to examine the expression of a gene-specific pre-mRNA or mRNA in a cervical carcinoma cell infected with Adenovirus serotype 5.

### Example 3

The purpose of this example is to describe the experimental workflow for the work described herein.

The experimental workflow commenced with a robust 3-tier approach for selection of heterologous introns. Following Tier 1, the initial selection as described in Example 2, heterologous intron candidates were subjected to multiple in silico analyses (Tier 2). Specifically, splice site strength was estimated with a computationally derived probability of use function based upon the principle of maximum entropy (See Yeo & Burge, 2004, J. of Computational Biology 11(2-3); Shepard et al., 2011, Nucl. Acids Res. 39(20); and the MaxEntScan::score5ss and MaxEntScan::score3ss tools available from the Christopher Burge Lab MIT website [software tab] for human 5′ splice sites and human 3′ splice sites, respectively). Heterologous intron candidates of sufficient splice site strength were analyzed for RNA splice site secondary structure as the final tier (Tier 3) of in silico analysis (See Shepard & Hertel, 2008, RNA (14); Zuker, 2003, Nucl. Acids Res. 31(13)).

Selected heterologous intron candidates were then inserted into the previously identified consensus or “very strong” splice sites within the coding sequence of the Cap VP3 region of a Trans plasmid, which expresses AAV8 Rep and AAV8 Cap proteins, via conventional molecular techniques known in the art.

Once generated, these intron-modified Trans plasmids were utilized to produce rAAV via triple transfection of Cis, intron-modified Trans and pAdHelper plasmids into Hek293 cells. Following triple transfection, cells were cultured for 3-5 days in standard conditions with metabolite and pH adjustments performed at least every 24 hours. Cellular supernatant was harvested after culture for 3-5 days and clarified through a standard laboratory filter. Once clarified, the supernatant was incubated with an affinity resin for capture and purification. Purified rAAV was then concentrated and buffer exchanged to ameliorate qPCR inhibition by matrix effects.

Following rAAV production and affinity capture, purified rAAV vector material was analyzed for critical quality attributes by multiple methods. These methods may include assessment of vector genome integrity by alkaline gel DNA electrophoresis, assessment of vector capsid ratios by SDS-PAGE or Western blot, and quantification of viral titer by quantitative PCR (qPCR) utilizing primer/probes designed to detect the transgene of interest or other selected molecular features present in the viral genome. qPCR was also utilized to quantitate Rep and Cap reverse packaged DNA present in purified viral preparations.

### Example 4

The purpose of this example is to demonstrate that levels of reverse packaged cap DNA and reverse packaged rep DNA are reduced when the AAV8 Cap coding sequence of a Trans plasmid is modified via the insertion of one or more heterologous excisable intron sequences in the VP3 region (an approach termed herein as “packtron”). Moreover, this example shows that rAAV genome packaging is significantly and surprisingly increased when using a packtron plasmid for the production of rAAV.

In this example, rAAV vector was produced via triple transfection of Hek293 cells with a Cis plasmid encoding human Factor IX (hFIX), various versions of packtron AAV8 Cap Trans plasmid differing in heterologous intron and location of insertion, and pAdHelper plasmid. Transfected Hek293 cells were cultured for 3-5 days, cellular supernatant was then harvested and viral particles were purified by affinity resin capture followed by concentration and buffer exchange. Viral particles were then treated with DNase to remove non-encapsidated DNA and analyzed by qPCR with primer/probes designed to detect AAV8 cap DNA, rep DNA, or specific molecular elements within the hFIX genome construct.

The packtron AAV8 Cap Trans plasmids used in this example were created with the following heterologous intron insertions: ALDOA (SEQ ID NO: 14) at location C-1, COL1A2 (SEQ ID NO: 2) at location C-1, COL1A2 (SEQ ID NO: 2) at location A-11, SPARC (SEQ ID NO: 5) at location C-1, SPARC (SEQ ID NO: 5) at location A-11, GNAS (SEQ ID NO: 20) at location C-1, GNAS (SEQ ID NO: 20) at location A-11, ENO1 (SEQ ID NO: 9) at location C-1, and ENO1 (SEQ ID NO: 9) at location A-11.

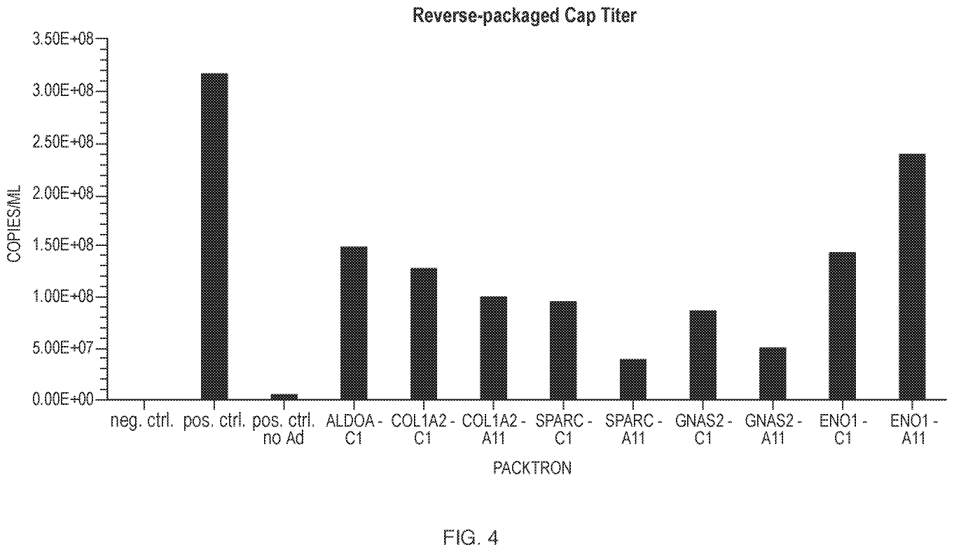

As shown in FIG. 4, 2-fold to 8-fold reductions of reverse packaged AAV8 cap DNA were observed across the majority of rAAV8-hFIX products made with the use of packtron Trans plasmids when compared to a rAAV8-hFIX product made with a standard Trans plasmid. The levels of observed reduction of reverse packaged cap DNA appeared to be dependent, in part, on the specific heterologous intron utilized as well as location of the inserted intron.

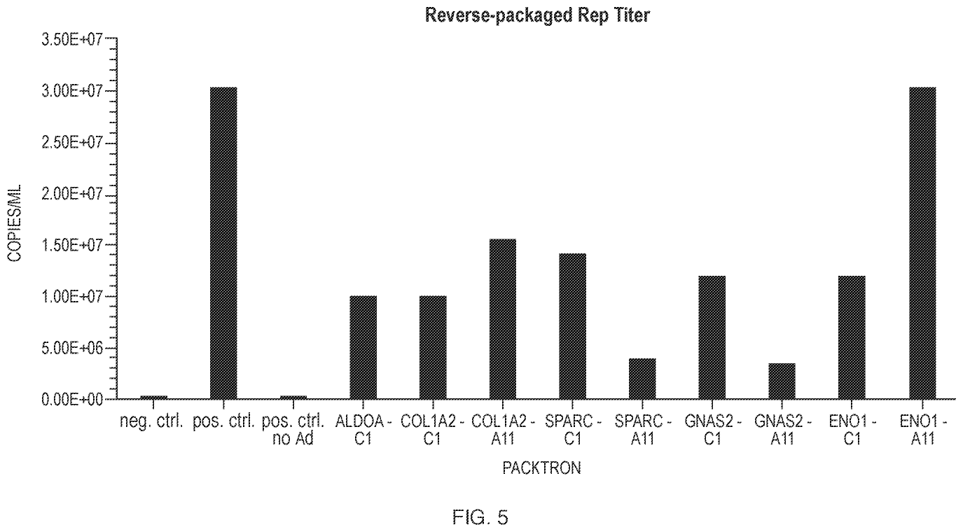

As shown in FIG. 5, 2-fold to 8.5-fold reductions of reverse packaged AAV8 rep DNA were observed across the majority of rAAV8-hFIX products made with the use of packtron Trans plasmids when compared to a rAAV8-hFIX product made with a standard Trans plasmid. The levels of observed reduction of reverse packaged rep DNA appeared to be dependent, in part, on the specific heterologous intron utilized as well as location of the inserted intron.

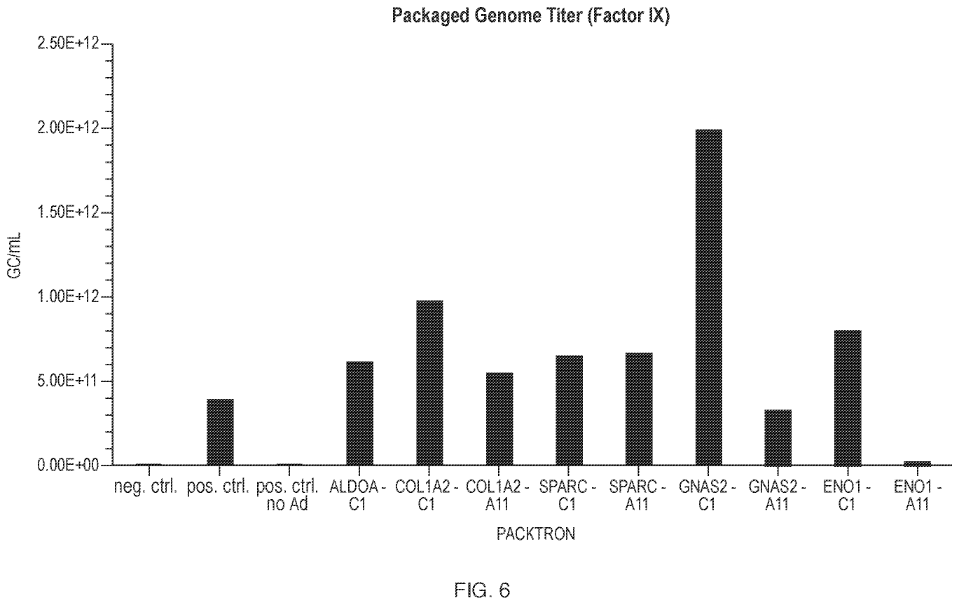

In addition to the reductions in reverse packaged cap and rep DNA, the inventors surprisingly observed 1.5-fold to 5-fold increases of packaged hFIX vector genome DNA in a majority of rAAV8-hFIX products made with the use of packtron Trans plasmids when compared to a rAAV8-hFIX product made with a standard Trans plasmid. See FIG. 6. The levels of observed increase of packaged hFIX DNA appeared to be dependent, in part, on the specific heterologous intron utilized as well as location of the inserted intron.

This example indicates that inclusion of heterologous introns into consensus and “very strong” splice donor and acceptor motifs found within the AAV8 VP3 ORF reduces the levels of reverse packaged cap and rep DNA sequences derived from the transfected Trans plasmid and increases the levels of packaged vector genome sequence.

### Example 5

The purpose of this example is to demonstrate that typical Cap expression and stoichiometry is maintained when a packtron plasmid is employed for rAAV production.

In this example, rAAV vector was produced via triple transfection of Hek293 cells with a Cis plasmid encoding human Factor IX (hFIX), various versions of packtron AAV8 Cap Trans plasmid differing in heterologous intron and location of insertion (as shown in the prior example), and pAdHelper plasmid. Transfected Hek293 cells were cultured for 3-5 days and harvested from the cellular supernatant by centrifugal pelleting. Once isolated, pelleted Hek293 cells were lysed and analyzed by Western blot utilizing anti-AAV capsid antibodies.

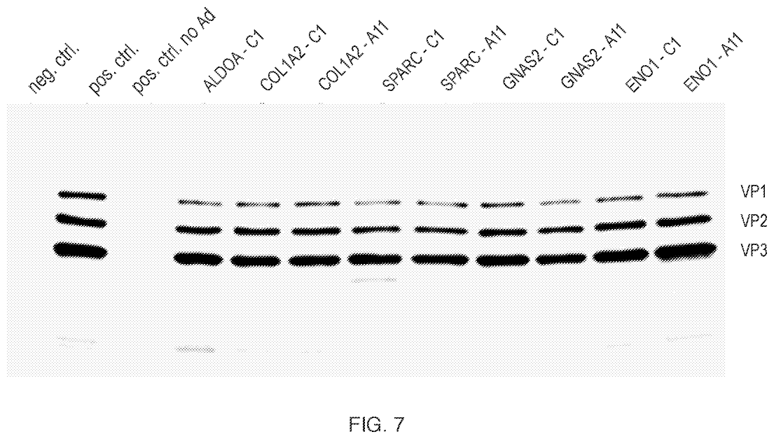

As shown in FIG. 7, the ratio of intracellular AAV8 capsid protein expression of VP1, VP2, and VP3 from each packtron plasmid was observed to be similar to protein expression from a control Trans plasmid. These data appear to be consistent with previously reported capsid ratios of 1:1:10 of VP1, VP2 and VP3, respectively.

This example indicates that insertion of a heterologous intron into the AAV8 VP3 ORF did not significantly alter the expression level or splicing of the isoforms of AAV8 Cap proteins.

### Example 6

The purpose of this example was to evaluate whether the packtron plasmid can convey benefits to other rAAV8 products having markedly different genome characteristics. Specifically, three genomes with different sizes, regulatory elements, and transgenes were analyzed.

In this example, rAAV vector was produced via triple transfection of Hek293 cells with a Cis plasmid encoding hFIX, eGFP, or mCherry, two versions of packtron AAV8 Trans plasmid differing in heterologous intron and location of insertion, and pAdHelper plasmid. Transfected Hek293 cells were cultured for 3-5 days, cellular supernatant was then harvested and viral particles were purified by affinity resin capture followed by concentration and buffer exchange. Viral particles were then treated with DNase to remove non-encapsidated DNA and analyzed by qPCR with primer/probes designed to detect AAV8 cap DNA, rep DNA, or specific molecular elements shared between the three genome constructs.

The packtron AAV8 Cap Trans plasmids used in this example were created with the following heterologous intron insertions: SPARC (SEQ ID NO: 5) at location A-11 and GNAS (SEQ ID NO: 20) at location C-1.

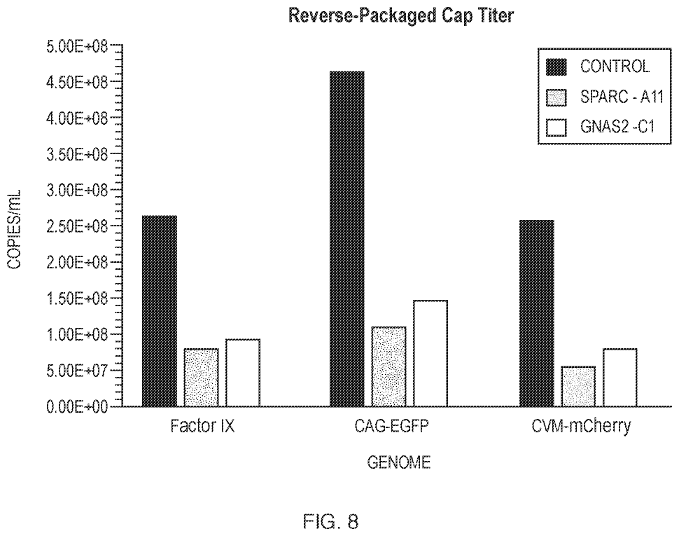

As shown in FIG. 8, reductions of reverse packaged AAV8 cap DNA were observed with the use of packtron Trans plasmids when compared to the standard Trans plasmid for all genomes analyzed. The levels of observed reduction of reverse packaged cap DNA appeared to be dependent, in part, on the specific heterologous intron utilized as well as location of the inserted intron.

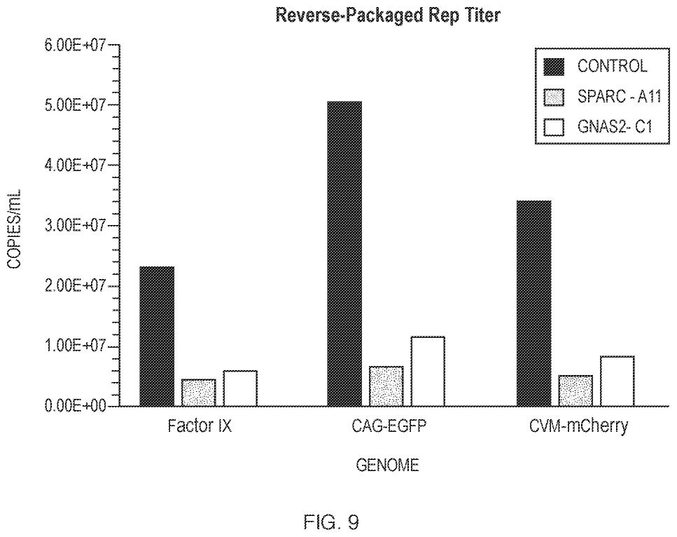

As shown in FIG. 9, reductions of reverse packaged AAV8 rep DNA were observed with the use of packtron Trans plasmids when compared to the standard Trans plasmid for all genomes analyzed. The levels of observed reduction of reverse packaged rep DNA appeared to be dependent, in part, on the specific heterologous intron utilized as well as location of the inserted intron.

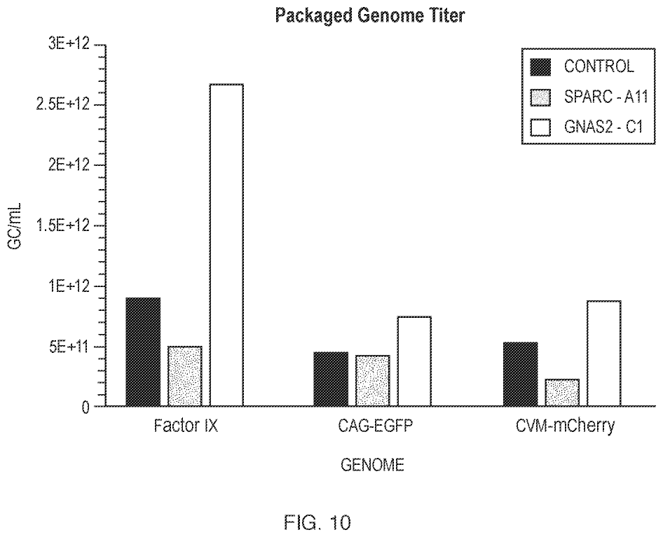

As shown in FIG. 10, increases of packaged vector genome DNA were observed with the use of a packtron Trans plasmid comprising a GNAS2 intron when compared to the standard Trans plasmid for all three genomes analyzed.

This example indicates that inclusion of heterologous introns into consensus and “very strong” splice donor and acceptor motifs found within the AAV8 VP3 ORF reduces the levels of reverse packaged cap and rep sequences derived from the transfected Trans plasmid and can increase the levels of packaged vector genome sequence across three rAAV8 products having different genome characteristics.

### Example 7

As described in Examples 4 and 6, use of a packtron plasmid was beneficial to the production of rAAV8 products. The purpose of this example was to evaluate whether the packtron plasmid can convey similar benefits to rAAV9 products.

In this example, the AAV9 Cap VP3 ORF underwent in silico analysis and insertion of heterologous introns as described in Examples 1, 2 and 3. rAAV vector was produced via triple transfection of Hek293 cells with a Cis plasmid encoding hFIX, various versions of packtron AAV9 Cap Trans plasmid differing in heterologous intron and location of insertion, and pAdHelper plasmid. Transfected Hek293 cells were cultured for 3-5 days, cellular supernatant was then harvested and viral particles were purified by affinity resin capture followed by concentration and buffer exchange. Viral particles were then treated with DNase to remove non-encapsidated DNA and analyzed by qPCR with primer/probes designed to detect AAV9 cap DNA, rep DNA, or specific molecular elements within the hFIX genome construct.

The packtron AAV9 Cap Trans plasmids used in this example were created with the following heterologous intron insertions: ALDOA (SEQ ID NO: 14) at location A-4 (a CAG/G junction in AAV9 VP3), COL1A2 (SEQ ID NO: 2) at location A-4, COL1A2 (SEQ ID NO: 2) at location A-5 (a CAG/G junction in AAV9 VP3), SPARC (SEQ ID NO: 5) at location A-4, SPARC (SEQ ID NO: 5) at location A-5, GNAS (SEQ ID NO: 20) at location A-4, GNAS (SEQ ID NO: 20) at location A-5, ENO1 (SEQ ID NO: 9) at location A-4, and ENO1 (SEQ ID NO: 9) at location A-5.

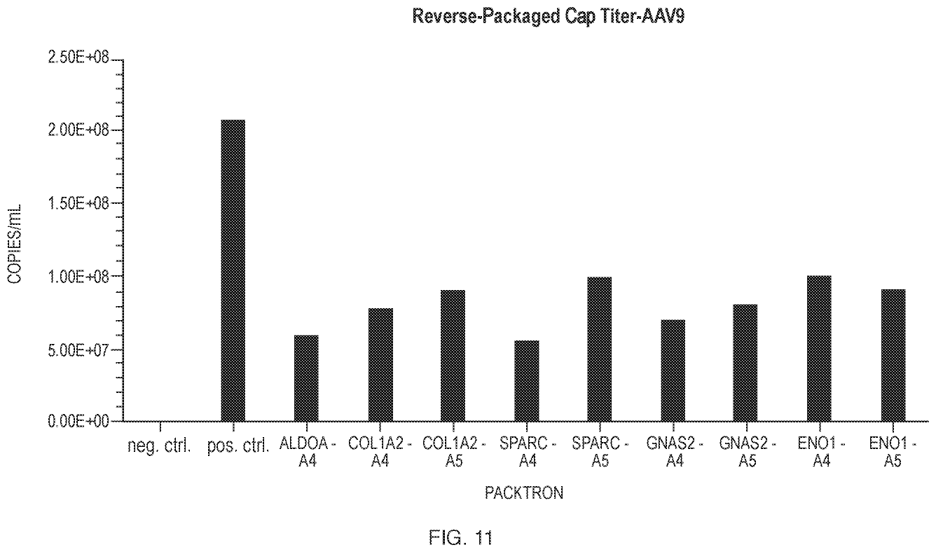

As shown in FIG. 11, reductions in reverse packaged AAV9 cap DNA were observed across all rAAV9-hFIX products with the use of packtron Trans plasmids when compared to the standard Trans plasmid. The levels of observed reduction of reverse packaged cap DNA appeared to be dependent, in part, on the specific heterologous intron utilized as well as location of the inserted intron.

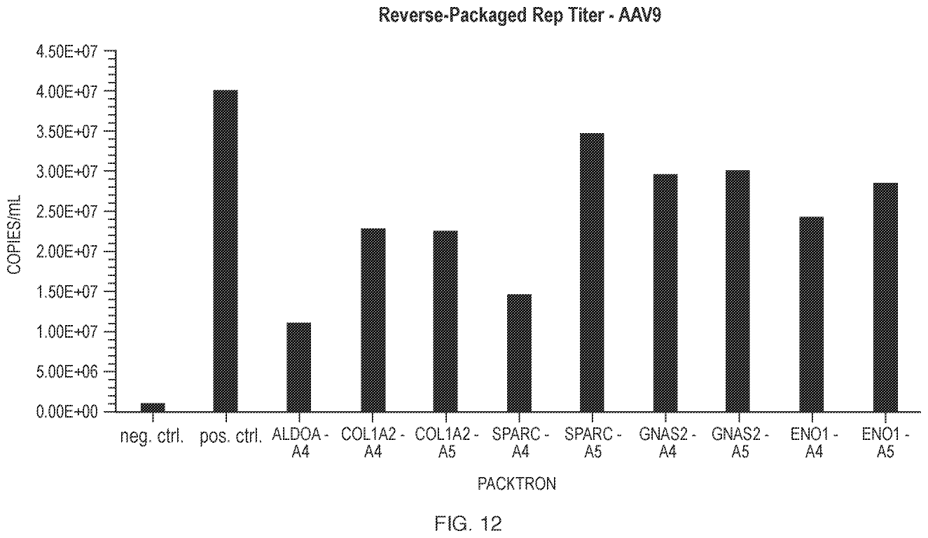

As shown in FIG. 12, reductions in reverse packaged AAV9 rep DNA were observed across all rAAV9-hFIX products with the use of packtron Trans plasmids when compared to the standard Trans plasmid. The levels of observed reduction of reverse packaged rep DNA appeared to be dependent, in part, on the specific heterologous intron utilized as well as location of the inserted intron.

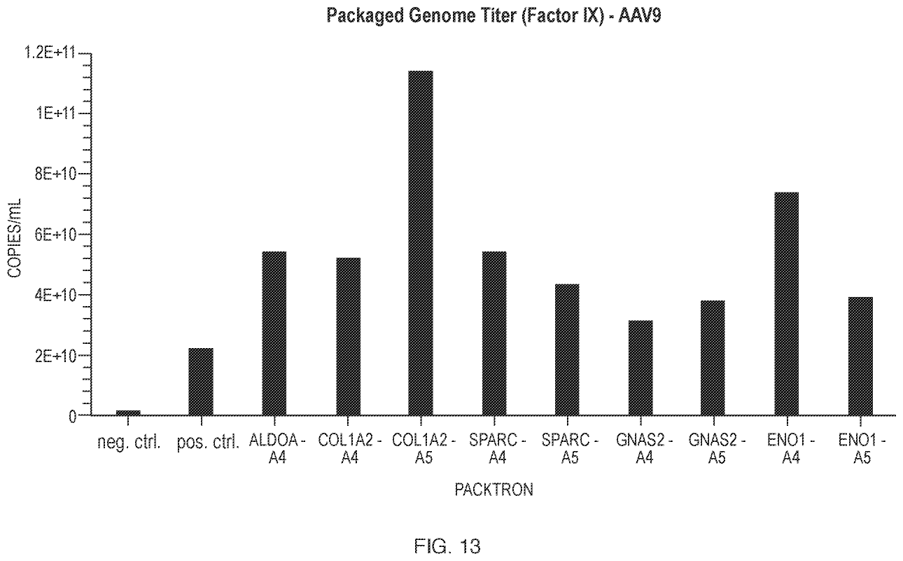

As shown in FIG. 13, increases of packaged hFIX vector genome DNA were observed across all rAAV9-hFIX products with the use of packtron Trans plasmids when compared to the standard Trans plasmid. The levels of observed increase of packaged hFIX DNA appeared to be dependent, in part, on the specific heterologous intron utilized as well as location of the inserted intron.

This example indicates that inclusion of heterologous introns into consensus and “very strong” splice donor and acceptor motifs found within the AAV9 VP3 ORF reduces the levels of reverse packaged AAV9 Rep and Cap sequences derived from the transfected Trans plasmid and increases the levels of packaged vector genome sequence.

While the invention described in these examples is illustrated in connection with representative AAV8 and AAV9 Cap coding sequences, the approach can be applied to any other AAV Cap coding sequence (and accordingly any capsid serotype-containing rAAV) using the disclosures of the present application.

### Numbered Embodiments

Embodiment P1: A recombinant nucleic acid construct comprising an AAV Rep coding sequence and an AAV Cap coding sequence,

wherein said AAV Cap coding sequence has been modified via the insertion of one or more heterologous excisable intron sequences in the VP3 region of said AAV Cap coding sequence, and

wherein the total length of the one or more heterologous excisable intron sequences together is at least 1 kb.

Embodiment P2: The recombinant nucleic acid construct of embodiment P1, wherein the AAV Cap coding sequence encodes a capsid protein of serotype AAV1, AAV2, AAV3, AAV4, AAV5, AAV6, AAV7, AAV9, AAV10, AAV11, AAV12, AAVrh10, AAVhu37, or an engineered variant thereof.

Embodiment P3: The recombinant nucleic acid construct of embodiment P1, wherein the AAV Cap coding sequence encodes a capsid protein of serotype of AAV8.

Embodiment P4: The recombinant nucleic acid construct of embodiment P1, wherein the AAV Cap coding sequence encodes a capsid protein of serotype of AAV9.

Embodiment P5: The recombinant nucleic acid construct of any one of embodiments P1-P4, wherein the recombinant nucleic acid construct further comprises one or more nucleic acid sequences selected from a promoter, an AAV intron, and a coding sequence for a selectable marker.

Embodiment P6: The recombinant nucleic acid construct of any one of embodiments P1-P5, wherein a single heterologous excisable intron sequence is inserted into the VP3 region of an AAV Cap coding sequence.

Embodiment P7: The recombinant nucleic acid construct of any one of embodiments P1-P5, wherein at least two heterologous excisable intron sequences are inserted into the VP3 region of an AAV Cap coding sequence.

Embodiment P8: The recombinant nucleic acid construct of any one of embodiments P1-P5, wherein the one or more heterologous excisable intron sequences comprise at least one splice donor and at least one splice acceptor site.

Embodiment P9: The recombinant nucleic acid construct of any one of embodiments P1-P5, wherein the one or more heterologous excisable intron sequences comprise at least one splice donor and at least one splice acceptor site.

Embodiment P10: The recombinant nucleic acid construct of any one of embodiments P1-P5, wherein the total length of the one or more heterologous excisable intron sequences together is at least 1.5 kb.

Embodiment P11: The recombinant nucleic acid construct of any one of embodiments P1-P5, wherein the total length of the one or more heterologous excisable intron sequences together is at least 2.0 kb.

Embodiment P12: The recombinant nucleic acid construct of any one of embodiments P1-P5, wherein the total length of the one or more heterologous excisable intron sequences together is at least 2.5 kb.

Embodiment P13: The recombinant nucleic acid construct of any one of embodiments P1-P5, wherein the total length of the one or more heterologous excisable intron sequences together is at least 3.0 kb.

Embodiment P14: The recombinant nucleic acid construct of any one of embodiments P1-P5, wherein the total length of the one or more heterologous excisable intron sequences together is at least 3.5 kb.

Embodiment P15: The recombinant nucleic acid construct of any one of embodiments P1-P5, wherein the total length of the one or more heterologous excisable intron sequences together is at least 4.0 kb.

Embodiment P16: The recombinant nucleic acid construct of any one of embodiments P1-P5, wherein the total length of the one or more heterologous excisable intron sequences together is 1.0 kb to 5.0 kb.

Embodiment P17: The recombinant nucleic acid construct of any one of embodiments P1-P5, wherein the total length of the one or more heterologous excisable intron sequences together is 2.0 kb to 3.5 kb.

Embodiment P18: The recombinant nucleic acid construct of any one of embodiments P1-P5, wherein the total length of the one or more heterologous excisable intron sequences together is about 2.0 kb.

Embodiment P19: The recombinant nucleic acid construct of any one of embodiments P1-P5, wherein the total length of the one or more heterologous excisable intron sequences together is about 2.5 kb.

Embodiment P20: The recombinant nucleic acid construct of any one of embodiments P1-P5, wherein the total length of the one or more heterologous excisable intron sequences together is about 3.0 kb.

Embodiment P21: The recombinant nucleic acid construct of any one of embodiments P1-P5, wherein the total length of the one or more heterologous excisable intron sequences together is about 3.5 kb.

Embodiment P22: The recombinant nucleic acid construct of any one of embodiments P1-P5, wherein the one or more heterologous excisable intron sequences is selected from an intron of eukaryotic translation initiation factor 2, subunit 1 (EIF2S1), collagen type I alpha 2 chain (COL1A2), secreted protein acidic and rich in cysteine (SPARC), signal transducer and activator of transcription 3 (STATS), enolase 1 (ENO1), pyruvate kinase (PKM), aldolase, fructose-bisphosphate A (ALDOA), Y-box binding protein 1 (YBX1), guanine nucleotide binding protein {G protein}, beta polypeptide 2-like 1 (GNB2L1), ribosomal protein S3 (RPS3), GNAS complex locus (GNAS), filamin A (FLNA), transferrin receptor (TFRC), polyA binding protein cytoplasmic 1 (PABPC1), ubiquitin like modifier activating enzyme 1 (UBA1), calnexin (CANX), and lactate dehydrogenase A (LDHA).

Embodiment P23: The recombinant nucleic acid construct of any one of embodiments P1-P5, wherein the one or more heterologous excisable intron sequences is selected from a sequence which is at least 80%, 85%, 90%, 91%, 92%, 93%, 94%, 95%, 96%, 97%, 98%, 99% or more identical to any one of SEQ ID NOs: 1-31.

Embodiment P24: The recombinant nucleic acid construct of any one of embodiments P1-P5, wherein the one or more heterologous excisable intron sequences comprise a sequence selected from SEQ ID NOs: 1-31.

Embodiment P25: The recombinant nucleic acid construct of any one of embodiments P1-P5, wherein the one or more heterologous excisable intron sequences consist of a sequence selected from SEQ ID NOs: 1-31.

Embodiment P26: The recombinant nucleic acid construct of any one of embodiments P1-P5, wherein the one or more heterologous excisable intron sequences is inserted at a location of cap VP3 having a splice donor:acceptor junction sequence of CAG/G (SEQ ID NO: 32).

Embodiment P27: The recombinant nucleic acid construct of any one of embodiments P1-P5, wherein the one or more heterologous excisable intron sequences is inserted at a location of cap VP3 having a splice donor:acceptor junction sequence of CAG/CTG (SEQ ID NO: 33).

Embodiment P28: The recombinant nucleic acid construct of any one of embodiments P1-P5, wherein the one or more heterologous excisable intron sequences is inserted at a location of cap VP3 having a splice donor:acceptor junction sequence of CAG/GTG (SEQ ID NO: 34).

Embodiment P29: A vector comprising a recombinant nucleic acid construct of any of embodiments P1-P28.

Embodiment P30: The vector of embodiment P29, wherein said vector is a plasmid.

Embodiment P31: A host cell comprising a recombinant nucleic acid construct of any of embodiments P1-P28.

Embodiment P32: The host cell of embodiment P31, wherein the recombinant nucleic acid construct is present on a vector.

Embodiment P33: The host cell of embodiment P32, wherein the vector is a plasmid.

Embodiment P34: The host cell of any of embodiments P31-P33, wherein the host cell further comprises a plasmid containing one or more adenoviral helper genes.

Embodiment P35: The host cell of any of embodiments P31-P34, wherein the host cell further comprises a plasmid comprising a 5′-inverted terminal repeat (5′-ITR) sequence, a promoter, a transgene coding sequence, and a 3′-inverted terminal repeat (3′-ITR) sequence.

Embodiment P36: The host cell of any of embodiments P31-P33, wherein the host cell further comprises:

a plasmid containing one or more adenoviral helper genes; and

a plasmid comprising a 5′-inverted terminal repeat (5′-ITR) sequence, a promoter, a transgene coding sequence, and a 3′-inverted terminal repeat (3′-ITR) sequence.

Embodiment P37: The host cell of any of embodiments P35-P36, wherein the transgene coding sequence is a native coding sequence.

Embodiment P38: The host cell of any of embodiments P35-P36, wherein the transgene coding sequence is a codon-optimized coding sequence.

Embodiment P39: The host cell of any of embodiments P35-P36, wherein the transgene is selected from ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit alpha (PCCA), propionyl-CoA carboxylase subunit beta (PCCB), survival motor neuron (SMN), iduronate-2-sulfatase (IDS), alpha-1-iduronidase (IDUA), tripeptidyl peptidase 1 (TPP1), low-density lipoprotein receptor (LDLR), myotubularin 1, acid alpha-glucosidase (GAA), dystrophia myotonica-protein kinase (DMPK), N-sulfoglucosamine sulfohydrolase (SGSH), fibroblast growth factor-4 (FGF-4), rab escort protein 1 (REP1), carbamoyl synthetase 1 (CPS1), argininosuccinate lyase (ASL), arginase, fumarylacetate hydrolase, alpha-1 antitrypsin, methyl malonyl CoA mutase, a cystic fibrosis transmembrane conductance regulator (CFTR) protein, minidystrophin, and microdystrophin.

Embodiment P40: The host cell of any of embodiments P31-P39, wherein said host cell is selected from a Hek293, HeLa, Cos-7, A549, BHK, Vero, RD, ARPE-19, or MRC-5 cell.

Embodiment P41: The host cell of any of embodiments P31-P39, wherein said host cell is a Hek293 cell.

Embodiment P42: A method of producing a preparation of recombinant AAV (rAAV), said method comprising culturing a host cell of any of embodiments P31-P41 under suitable conditions that promote the production of rAAV.

Embodiment P43: The method of embodiment P42, wherein the preparation of rAAV contains reduced levels of cap DNA compared to a corresponding preparation of rAAV produced using an unmodified AAV Cap coding sequence.

Embodiment P44: The method of embodiment P42, wherein the preparation of rAAV contains reduced levels of rep DNA compared to a corresponding preparation of rAAV produced using an unmodified AAV Cap coding sequence.

Embodiment P45: The method of embodiment P42, wherein the preparation of rAAV contains reduced levels of rep DNA and cap DNA compared to a corresponding preparation of rAAV produced using an unmodified AAV Cap coding sequence.

Embodiment P46: An rAAV produced by the method of any of embodiments P42-P45.

Embodiment P47: A pharmaceutical composition comprising an rAAV of embodiment P46 and a pharmaceutically acceptable carrier.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

1. A recombinant nucleic acid construct comprising an AAV Rep coding sequence and an AAV Cap coding sequence,
wherein said AAV Cap coding sequence has been modified via the insertion of one or more heterologous excisable intron sequences in the VP3 region of said AAV Cap coding sequence, and
wherein the total length of the one or more heterologous excisable intron sequences together is at least 1 kb.

2. The recombinant nucleic acid construct of claim 1, wherein the AAV Cap coding sequence encodes a capsid protein of serotype AAV8, AAV9, AAV1, AAV2, AAV3, AAV4, AAV5, AAV6, AAV7, AAV10, AAV11, AAV12, AAVrh10, AAVhu37, or an engineered variant thereof.

3-4. (canceled)

5. The recombinant nucleic acid construct of claim 1, wherein the recombinant nucleic acid construct further comprises one or more nucleic acid sequences selected from a promoter, an AAV intron, and a coding sequence for a selectable marker.

6-7. (canceled)

8. The recombinant nucleic acid construct of claim 1, wherein the one or more heterologous excisable intron sequences comprise at least one splice donor and at least one splice acceptor site.

9. The recombinant nucleic acid construct of claim 1, wherein the total length of the one or more heterologous excisable intron sequences together is at least 1.5 kb.

10-14. (canceled)

15. The recombinant nucleic acid construct of claim 1, wherein the total length of the one or more heterologous excisable intron sequences together is 1.0 kb to 5.0 kb.

16-20. (canceled)

21. The recombinant nucleic acid construct of claim 1, wherein the one or more heterologous excisable intron sequences is an intron sequence from a gene encoding a protein selected from eukaryotic translation initiation factor 2, subunit 1 (EIF2S1); collagen type I alpha 2 chain (COL1A2); secreted protein acidic and rich in cysteine (SPARC); signal transducer and activator of transcription 3 (STATS); enolase 1 (ENO1); pyruvate kinase (PKM); aldolase, fructose-bisphosphate A (ALDOA); Y-box binding protein 1 (YBX1); guanine nucleotide binding protein {G protein}, beta polypeptide 2-like 1 (GNB2L1); ribosomal protein S3 (RPS3); GNAS complex locus (GNAS); filamin A (FLNA); transferrin receptor (TFRC); polyA binding protein cytoplasmic 1 (PABPC1); ubiquitin like modifier activating enzyme 1 (UBA1); calnexin (CANX); and lactate dehydrogenase A (LDHA).

22. The recombinant nucleic acid construct of claim 1, wherein the one or more heterologous excisable intron sequences comprise a sequence selected from a sequence which is at least 80%, 85%, 90%, 91%, 92%, 93%, 94%, 95%, 96%, 97%, 98%, 99% or more identical to any one of SEQ ID NOs: 5, 1-4, and 6-31.

23-24. (canceled)

25. The recombinant nucleic acid construct of claim 1, wherein the one or more heterologous excisable intron sequences is inserted at a location of cap VP3 having a exon splice donor/exon splice acceptor sequence selected from CAG/G (SEQ ID NO: 32), CAG/CTG (SEQ ID NO: 33), and CAG/GTG (SEQ ID NO: 34).

26-27. (canceled)

28. A vector comprising the recombinant nucleic acid construct of claim 1.

29. (canceled)

30. A host cell comprising the recombinant nucleic acid construct of claim 1.

31-34. (canceled)

35. The host cell of claim 30, wherein the host cell further comprises:
a. a plasmid containing one or more adenoviral helper genes; and
b. a plasmid comprising a 5′-inverted terminal repeat (5′-ITR) sequence, a promoter, a transgene coding sequence, and a 3′-inverted terminal repeat (3′-ITR) sequence.

36. The host cell of claim 35, wherein the transgene coding sequence is a native coding sequence or a codon-optimized coding sequence.

37. (canceled)

38. The host cell of claim 35, wherein the transgene is selected from ornithine transcarbamylase (OTC), glucose 6-phosphatase (G6Pase), factor VIII, factor IX, ATP7B, phenylalanine hydroxylase (PAH), argininosuccinate synthetase, cyclin-dependent kinase-like 5 (CDKL5), propionyl-CoA carboxylase subunit alpha (PCCA), propionyl-CoA carboxylase subunit beta (PCCB), survival motor neuron (SMN), iduronate-2-sulfatase (IDS), alpha-1-iduronidase (IDUA), tripeptidyl peptidase 1 (TPP1), low-density lipoprotein receptor (LDLR), myotubularin 1, acid alpha-glucosidase (GAA), dystrophia myotonica-protein kinase (DMPK), N-sulfoglucosamine sulfohydrolase (SGSH), fibroblast growth factor-4 (FGF-4), rab escort protein 1 (REP1), carbamoyl synthetase 1 (CPS1), argininosuccinate lyase (ASL), arginase, fumarylacetate hydrolase, alpha-1 antitrypsin, methyl malonyl CoA mutase, a cystic fibrosis transmembrane conductance regulator (CFTR) protein, minidystrophin, and microdystrophin.

39. The host cell of claim 38, wherein said host cell is selected from a Hek293, HeLa, Cos-7, A549, BHK, Vero, RD, ARPE-19, and MRC-5 cell.

40. (canceled)

41. A method of producing a preparation of recombinant AAV (rAAV), said method comprising culturing the host cell of claim 30 under suitable conditions that promote the production of rAAV, wherein the preparation of rAAV contains reduced levels of cap DNA or rep DNA or both cap and rep DNA compared to a corresponding preparation of rAAV produced using an unmodified AAV Cap coding sequence.

42-44. (canceled)

45. An rAAV produced by the method of claim 44.

46. A pharmaceutical composition comprising the rAAV of claim 45 and a pharmaceutically acceptable carrier.

47. The recombinant nucleic acid construct of claim 1,
wherein (i) said AAV Cap coding sequence encodes a capsid protein of serotype AAV8, and
the one or more heterologous excisable intron sequences are selected from the group consisting of SEQ ID NO: 14 inserted at location C-1, SEQ ID NO: 2 inserted at location C-1, SEQ ID NO: 2 inserted at location A-11, SEQ ID NO: 5 inserted at location C-1, SEQ ID NO: 5 inserted at location A-11, SEQ ID NO: 20 inserted at location C-1, SEQ ID NO: 20 inserted at location A-11, SEQ ID NO: 9 inserted at location C-1, SEQ ID NO: 9 inserted at location A-11, and any combination(s) thereof; or
(ii) said AAV Cap coding sequence encodes a capsid protein of serotype AAV9 and the one or more heterologous excisable intron sequences are selected from the group consisting of SEQ ID NO: 14 inserted at location A-4, SEQ ID NO: 2 inserted at location A-4, SEQ ID NO: 2 inserted at location A-5, SEQ ID NO: 5 inserted at location A-4, SEQ ID NO: 5 inserted at location A-5, SEQ ID NO: 20 inserted at location A-4, SEQ ID NO: 20 inserted at location A-5, SEQ ID NO: 9 inserted at location A-4, SEQ ID NO: 9 inserted at location A-5, and any combination(s) thereof.

48. (canceled)

49. The recombinant nucleic acid construct of claim 47, wherein the AAV Cap coding sequence before modification comprises a nucleotide sequence that is at least 90%, 91%, 92%, 93%, 94%, 95%, 96%, 97%, 98%, or 99% identical to the nucleotide sequence of SEQ ID NO: 38 or SEQ ID NO: 42.

50-54. (canceled)


SEQUENCE LISTING (44 sequences)

SEQ ID NO: 1 - DNA, 4310 bp, Homo sapiens
gtgactaatcccaagctcggtttctccaggaactcactttggtttgtgtccagtgacatgggtgttcgagagcgggtaggccccgggctaatacctccctttcccgtccctgcgcgggaagtggctggacagcaccgccaccacccgcgctgtcgtgggcctcctcgccggctccgtgtaggtcatcctgtgttccagaggcatcgttgttcgagcagaggaatctcgtgaaggaaagtgggccgagtggattgtacagcctctacaatatgccgaaagtgagaatgccatgacgatggggcggtctcaggagaacgctgtgacaccgtaactcttaaccccggctcaggacgctgagcactttgctttttccaggcttgagaattccatcttttcttccgccccaacagctctaccaaaaatttcccctgcgtctcagtggaaaccaaactggcctcttacctgagaaacctgtgggacgggcaccgtgttggaaagagcggggtccgagtagtttttcatctgataattgaatgagtcaccccttgaaccaaaactaaatgagacttgagtttattactgttactatcagtttgtcgtattttggcgacaatgagaatagaactgtgttaagcatatgttaggttaactatgttagttaagcataactaaaacctaagataaggatatattttcgggttaaatacacagtcatgctgaggtctctttatcctcaaaacttaggctttttaaatttttattaagcaaaaaacatggtttcttcgagaaaaagtatagcagagaagtctttatttcccctcaattgagttgttattcaaggaacaatctctgtcaatagtttgggggtgacttgtacagaactttgctacctattcttagttcatctggtgccaaaagatgtagttgtctcatgcacagtaatgtttgtagagattcggtgataactttctttttcaactataatgatgaaataaaagctttacttatgtagcactcctataccaagaactagcattcagagagcattcctaatacgtgttagatccaaattagaattatcacgagagttgtccccatcacataatatattttggaaaggaatgtgaacttctatatccattatagcagtttgaccatgcactgttttgagaaagaagtcctacctaccatttgtcatgcaaaataactgactatgcagggtatttgaaattagcctggagtttggttgatatattttgaaaggtggctgatactttttacatcagaacacataagagtattatagcatgttgtgatattgaggttctgtgggttaaagacattctggatttgatgtcggattacttgagtttagctcctacgctagtacttattaagcatttgttcttaaggaagtattataatttagtatttctgttccacctttcctgttcattgcttcactgtgccctttcatagtactttgtattgtaattatttgcttgctagtttgtttcccactctatacttaattacttaggtaagagtcttttttttttttcttttttcttttttctttttttttttgagacagagtctctgttgcccaggctggagtgtagtggcacgatctcagctcactgtagcctctgcccctgggttcaagggattctcctgcctcagccactatagtagtgggattacaggtgcctgccaccacacccagctaattttggtatttttagtagaaatggggtttcaccatgttgaccaggctggcctcgaactcctggtttcaagtgatccacccgcctcggcctcccaaagtgctgggattacaggtgtgagccactgcgcctggcctggtaagagtctttcttgttgtatcattagtgcttagtacatagtagctactgaaaaaaaaattaacaaattattatattacaaaatggaaataccttctttttctttcaatattttatgtttgactagcacataatggttttcaaaatgtttttacgtatacatgtgataccctcagtaaacctgtgatgtacatggcacagatgagaaactacagttcaaaggatgttatctttcccaagcagccacttctagtaagtagcagagcaaggactggaacacaaggctagagttgttccccgagtttactatgttaattgcctgatttgacacttcttgaggaccaaataaaataagaatgctgtgaaaaagataaagaggtgaagtcacctagtgtgacttcgatattgcggaatgatttttttaagttaaaaaaaaaatttaagttattagcatttcattaggcaatatgttcaaaaattgtcaaccaatatgcaggtttttgtggtatggattctttttgtacttaactactatctatctttactatgaataacataccatcaacagtattcctttacttaccctttatgttgcaaagagacttgtgcctcctgaagaaatagtattcatagtcatatcccttcaggattctcttacatgattgcccatgttttttaaccatcaaattaagccttgcttcccctaaaagcatcacctttactgtttggagaagcttggtaagtaaatgtggttttcactggtcaaatgtcttgcaactgttccatacatttctgtaacatagtttatttttatgcccaatttgaaactttttagctctcaaatttgagagctttacatacacatattcaacaacagtttccccacccccaccctcgaaaagccctgtttaaaaacgaaacaaacagtcagctgtttcttggtgggtaaggccattgaatgtctacttctctttatttgctaaatccctattttgtagggattttgtattccctacaaagaaatacatctctgctactgtcattaatatgctgctctttgttgtcaaacagatgagtactaatagtttgcacttattggtgttatgtactctggcactgaatatatattaatgttgctttttaagtaaaatgtttctaattataatatatttatgaatagttttgagagaatatagtcttactgataagaggacagactctggatccagacttcatggattcaaatcctgactcatcacctgttagcagtatgtgaaagaacaagtaacttaacctgtgcttcagttttttaatctataaaatggggagaactagggagaaagttgtaccacaaggagataggcaatggccagatcatataggattctgtaggtagtggaaagatacataagtcagtgggaagccactgagaagatataggcaggtgccaattaataagttattatatgaattttcacaaggttttcttaggcctttaagttctgttactacctacgttttgagcagtgacaacagtgaaggttaacagaaaaacatgaaaatcatcagccatgattagtttgagagcttccttttttaaatatgcttctacccaggctatgatttcaggcttgtggatcataaaaggagtagtgctgaaagtaggttgggtggatcaaagggaataattgtaaatatctttaatgcttggaggttaagagaaacttgaactttgtagtaatggagattaaagctattctcaagattaggtatacctattcaataaaagaaaggaatatttgggagtattggggataagagaagctagacattgtgaattcaaggaaaaagaattatgagaggaggatgcattttagtttattttgtgattaaaatttttttagtttttgttgttttgacatgtgacactgtaattttggctataataaatgacagaatgtatcatttggtatattggttagggtttaggtagaaaattttttcctttatatagtgagtggtagcgtatacaatgtttgctcacttcggcaaagagtagctttagttttctgattagttataatagtgatgcttcccatcatttgatatgccttaaagtagtattttactcttgaggtattcctttaatccttaggtcttggagtactttatagatggtcattattttttatgattcctctgaaactaaaggcagagaatcacccaagcattacaaaaatgttgagcaaaataaaaattaaagcttggttcctgaacattatctgttttctggtaccacttaagaagtttcaaaggatgagaagactaagactaataactatttttttcttcatcttttctttcagtggcaggatgtggaaattgattttttttttattttcaccttaactgaatacttacttaattcttttgtttaaattgcag

SEQ ID NO: 2 - DNA, 2646 bp, Homo sapiens
gtaagtgccttcagcttgtttgggggagactgggtagagaggttagatgggagggcaccctgccctgaaaaggaaaacctgtaacctgaattccaggtacacttggagggcagactctcaggcatgtgggaaaacgccggaattgataagaaacatggaaattactttaaaaaatgaaaacataaaagccttgccaaaagttagggaacttttcctctaagttcagagtgagacagttaactcggtctggctcctcagcttagtaacccccaaagggagcggaaggtctttttccctaaggatgagatattaacgaccaatgtggtggaggaagtcaagggcctgcaccccacaggccccataaccgcactgatgtccaccttgtaaaacttgaggcctgcgttagaaagcccttcaactgagtaatgtaaaactcacctcctaagagcttttatcttctgggcattgtaaggcttgtccggaggaggaggatgacgatgctgatatgatgatggttataaggcgccctctggaggaaggaaaatgaaagtacaggggacagggccttaagcagatggaatcccaattaaagcttctacggatttatacagattaatgatcagcatttctggttggagcctttcccagtggctagtcagtgaaccctggaaagaagaatggatgctacttggagtgggtacattctgaaaagtaatataagtgtctcaattcactttctagtcatggaaatggtaacattttttaactcaaatctgctctaaattttgtttgagcctgagaattacccctttgacatgttcccagtgataagcaaacattatgaacgcagcaagttgagaaatatcaacattgagatgagactcaagagaccggggtttttcccatgagtctgacaccaatttgctgcgtgactttgggcaagtcaaacggccttttctaaaatgtgagacagagattaaagggaccccaaggccactttccagctctaggttccatggccagactttcatgtcaacagagaatgaagaagatcagtccgttttcatcttgaaaatggctgccaaagtgctagacaaagatattgactagatgggggatggtattgtctgaccacacccagtactccaaaaagttgttccacccacacagcacggtgtctaccactgcataatttctaatgcatttgtgtgcttgtgtgtgtgtgtgtgtgtgtgtgtgtgtctgtgtgtctgtgtgtctcttcccccttcattcacttttagtatacatactgtggatactaaggagtaattgcagtgaacaaattcacattaccgagttcatatttttaatgagatcttgagagtgggaggaaagagtcggctcctagagaataaaatgaaggcagacttagggaaatttgaaggtacaaaggcaacttaccttctgatcaacagccaaccacagtctggaataaatgttatcaaacacacattcttcaaaatggtccatgtctgagtaattaaaaggcaaatttccaaaatcataaggacttccgttaatcaagtcaggcataattattcttcctactgatggacacaatgaagtaaacatatcattcttgtaatttaacagtaattctcgtaaattgcccttaaatgtcagtgctggatgtggtccaccctcctaaattgtgactgttgcaacagatgttctcacttcaaataacgcacttcttggccacctaattaaagcaatttttggggtgattcatcctactgcaagcttggccacacttgtatcctgtattaacctataatttttgtaccgtaggagaagaattcactctttaaggacttataacaattatggcaaaaggggggatagtacttttgtttattttttctattatttttcaagatctttaatccggtttttccatttatacaaaactctttctccgagacaaaaatgatacatattggtaaaatgatcttacctaatttaagtgaactaatttaaagcaaaattcagatgtctgaattaatccattttcatagttaataatgtgcaaattagaccttttgaaaaaggatattaagaatggtacaaactcaatgaagtactaggtaacttcaatgttttataaaaaagtaagtcagcttcaatgtttcataaaaaacaaattcaatatagaattttaaggtaacatactttcctaaattttaccttttttcgatatttaggtattaaaaatgatcaaaatcataaattatttcctcatcaatctactagtcttacattcagcgattcatctgtgcactttaccagcttaattgctaagcattcaaaatatccttcagacacattaatatttcacaacagttataaaatagtaaataattaataatttaattcaaaatacatttacatattaatattgcaaacaaatcaccctgctgatccctgccatacttttgacctgcataatttctaggtcattaaaatattcttaaaaaaatataattggtccttaattaggtaattcaattctataaacttgtttctctatttgttaattattgctattgatccatgaagtgatactaataattgtttcctactttttcttttttttttctacag

SEQ ID NO: 3 - DNA, 2935 bp, Homo sapiens
gtatgcttatctgtttatcttagccaaaaaaattgctaaataaatcattcattttatgtcacattttaccacgccatttatttagctacctaagttaacactcaatacttagattatataaaaacaactctttttgttttcaaatttatgaaaacataagttaaggagttcacttttctttacaaaagaaagattaattgatcttttatgattatatgatctttttgattatatgatcctcattaagatagatcatatacttatgtccaagaaataatctttggacatagtaaccataacttgggcaaatcaatttaatttaaaacagtaatcactctgattaattttttaatattctttaacattgcttagaattttaagcaacacttagaggcatagaactatttattaagttctctgaacttgttggaaaggatcaacaagttctatctagtccagctaactcattttaaaatgggagagtttaagcccttttctcaaagtcatccaggtaactaatgacataactagaactagatgccaggcaagatgtctaatatttgcttacatcatggtttatgtacctagtccttgaataaaccactcatttagtcaacagatattaatcagatgccttcaatgggccctaaactgtattaggaactggggaaattacaaggaatatgacagattctgatccttcctcaaggagttaacaatataggaaatgtttctttttctgaattttgaccaaaaaaatccttttttagtctattgattgtaaatctatatagaagagagtatgagtaaaaatctagcatttatgtcactcagtacaaatattcagcaccataccctatcagtggagcaccgcttagaaacattccctatatgatgatgatgatgatgatgactattaacaaatgaagcttctaacaagcattagagagaagtttgaagggaaaaatgctaatacgagcatgcaaaatgtatactagcatatatgaaatagaggggaaaactgccagaagtcaaagtgttaggttgattaagcactacagaatttaatgtatacacacacacgcaatttagtgattttaattaattgtttcaaaacaaaggtatttatctgcccaaagtcaacaaggtctttaaaatgtaaattttacctgggcagtgcacttagtgctctatcttcaaaagaagatgttctgctggagctaatggcccacagtaagctaatatactctaagggtgagataatattttctgtaaattaaaactcccacttgagaaataatgtacctttaattgacgacttctaattccctaattttttctggtagtttaaaatgttcatatctgaaatgaaaaagtagagtgtttcttttggctttgtttatattggatttttgaaattagctgtttcagctaatgctggacattagtcagttttaaagcagtacctacatctcaagaagaagcaagggggcggaaagtaaagagctactaaatgtcatttttaaaaagcccactaagctgggaaaattaatatggatttcagatacccctgttttcggaacatctgtcttggcataaagcagagtattttactttgaaatatcagtgaaatataatttaagcttgcacatccacacacatgcacagacatatgtaatcaacagatatctgtttcacaaatagggaagataggcagcaataaagtattaaaataatttccatgttggaaaatcaataactataaaaccccacagggttcttctctgaattaatgagtaatcacagcctccatgaaatacactacattttatgtaaatgaaattgttgcaaatacatgaaaaaataaatataattagaaattcatgatgtcaaagaaaattattttttaatgtatgcctaaaaagctattgtgatggaaaagtgacagtttcttttaatgtcagagcaatttctaaaaccaaatgaataattcttataattaaaatgacatacattttagataaaatccatgttatttcactctaggcattaatacagtaaggtaggtttgactgcagagtccccacagctgatgtcatgaacaaattacttgagactggtacatgaaatattttcagcattatgaggaacagaccctacggatgagcttacacaggcactgattactgcaaagaggagtcaagaaagtgtatttagcttacaaactattaacagcctgttttaccctacttttgtgctatggaaacaacaaaggggaaaacaatcttccatcatttgggccatattttcaacaataatatcatataatagactcttccagaaggctgtttcaataatgttttatttttccttcacccctcattacatccacttttgtttgacattttcatcagtcaccaataacccttagaggagcgataaggttataacaaacttctctctaatcattaagaaggacttttgattcttttcaatttatgtcctttgtggcaataaaaataccaatttcttagctaaatatgacataggaagatgacatatgatcaaagatatccaaatggacatgcttcatctgctgtatagaagacaattgtatattctgcacttctgcaaagactgattcacttcattgcatcagaacaatctcaatatgcccaattgtgcacaactttaaggaacctatctgccccgtctaattctcattgatttctgttgataaggattgggagaaaaggaaaagcaaagggagagaactagtgcaggaagtttgagtccttaaattcttccttgggaggaataaaaactatggaatcaaaccacaacaatggcactgctaagttggtcatatctgaccccagccaacaccatgacaacttatcagtgctaactgttgatatatctgctttctttacag

SEQ ID NO: 4 - DNA, 2063 bp, Homo sapiens
gtgagtgtccttgggcccttccagactcctgtcttggggagagagctctgagctggcactgatgctgtctctgccacccccatgctgggtgatcttgggcaaatctctctccctttctgagtctcggcatccccatttataaaatgaggtaattgtctatgagttggctcctctcaactctgagttcagggcactttctggggaaggactgagcgattatggaaggtcaacatcagggaagatttctggagtctctcactagcatctgagacaactgcatttcatttctctaatgaggaagaatttcttctctcaggctaagttgaatctttcttgcttcaacactgtaaagcaaaaatgatgtttgacctgggcctacaaaatattttgaaaatatctgattttgaatgctttaaaataagggcatgcctgtcggtttgctttgggccctgttacttggtactgtcatatcttcacccttttactccattacattcctatctgccctccgaagagagaggataagcaccctcttctggaaacctgggagtcagttgtctggggagggatgccagggtgtggtctgatctttggttagtgaccccagctgcaaactcagtggggccaagtgccttgttcagtatcctccctatcatgggagacccccaagaccacacctgctccccctccaaggcaacatccacactgtgggtcttgtgtattctggcacccgatggcaatgaggagagccactcacctacctcaggtgtacagggtgcagaaaagtatgtagggggtgggaaacaaagttctcctcgtgggaccccaggtatgaaattgagaaggtacagagtagaaaaggagggttgggggaagaagcatgggtaggaagtccctgggcagcaagactagagagcctgtcttaaggagtctttatgaagtggctttgctttccctgaggcatgttgcccactcacttcaactcctaaggtttttatcaccagatattctctcccagaccccagctgatgatgaaggtagcctcacagaggggactttccctggttcaatgatgagggaagaacagacccctaagcccccaggtgaccacactgccctggatgctggatgaggctctgacattgtggccttgagcaagtcccttctctccatgctgacctctaataagtccagtacaaagtgtgaacctgggtcaggcgcggtggctcacgcctgtaatcccagcactttgggaggccgagtagggaggatcacttgagctcaggagttcgagaccagtctgggcaacgtggcaaaaccttgtctctggaaaatatacaaaaattagctgggtatggtggcatgtgcctgtagtcccagctactcaggaggctgaggagggaggattggttgagtccaggaggctgaggctgcagtgagccgtgattgtgccactgcactccagcctgggcaacagagtgagaccctgtctaaaaaaaaaaaaaagaagaaaaaaagaaaaaagaaaagaaaaggaaaagaaaagtgtggacttggatgaaatcttcaggtccaacatttgggattctaagttccaaagaccaggttggaatcatttctaagaaggttctggtggttacacattcctgagtcctctactccccactccctgccaagctgggcctgtggatagatgtgatccctcagcctcccagcttcaaacacctgccagtggttgacgtgaacaacatgggctcagtctcagctaggatcacacccaaagcccagcacccagtaaggtgcaggagccatccatttccctgagcagagcagattaggctgaggaaagcagcagccatgcctttgcacaatgcatttctagggcattcttcccacacataatctcctctgctcattgtcctgtgaagaaactgtggcctggagaggttgagccactgtgccaaggccaccaatgcaggtggtatgtgggtgggtgggggcctggggtggggagcacggcccaggcagggtctgtgctgaccgcccttgtgtttggaacctag

SEQ ID NO: 5 - DNA, 2125 bp, Homo sapiens
gtaagaccccagaccctagagtgaacagagctcaaggctggcaggtgcactagctatggccagaaagcctctcagcagcctatactgctccaagccctggcatccacagtttgcctggggattggagcagaaggatgaggcatctggagaaaagatgtggacctgggacaaagaaaccgataagacactctcatgctgaggtgaaagtcagtaggagctcaaaatagctccataatcctgcaagtactaggcgtggatatctggataatgaaggagtgtgaattaagaaggagtaccaggctccaaggggtggcaggggacaaggttgggtcagccacacgccccctgtccttcagcagaacatccaggggcagagcagccacctggcactgtctaagccccctcctaaggctcagccccaatagggcccaactgaccctggaagttatccaaaaaagcctgtctattttgcaagcccccagtttgagggctcttgtcccttgtccaaacgagttatgaggccctgtgcaactgcactgccgaacaggcaggcagctggccagttagcaaatgcttatggagtgtgcattttgtgccctgcactattctaggcaggggattgaacagcagtcagagctggcatggtccttgccctcatggacttatactctgttcataacctgtcactaccttctgaacttctcttgtggtgatgaagtgagagcccctgctcagcctcagatggagcaagctacacctgcaccttcccagagtggttttttcttcgtccttgggttgtggaagcagagcatcacacagaggggaaaggaagggctgccctactcacatactcagggaacttcctctctaggatgttcacccctcgctctttgtccagcctgtgtgcctggagtctgccaaccctgccagtgatcctgagggctggggtctcctgggctctgggaatctcccggccacttctctcccaggcttttgccatggctgggatccaactgagtcactcattatggcagggaggggaaaagtcaaaggggaacatctggagctcaggcaaagcaatttgatcccactgcaacagagggcctggagggaggctttcagatggggtgcaagaacagcacatctgggaaaggggtccagcttgggcaaggggacccgcttcctcctcctcccatcccagggctgtaggtgaccttgcctgcatccctgcccctccctgggcctcagttttccaccagtacaatgaaggggaggagaatgttcctatcagttcaaacattgtgtgatttctttggtgagctgggtggggctgcgaggtctagaggttaagaagacaactggagtcacattgttccctggagatcctttgtggatctttagggacaagtagttgggggctctgggaaacaaagaaaaaaattatacacatgctctggagtctaaggccagcagggagaatagggagggaggacagtgggagagacatccaaagggcctccctctcagacattacaggatacacaagcaaagctctatgaagatggttagagctcccgttgaccctcactgccaatcccagtccctttccacattcctccccagaaggcagcactgtcaccagattggtgtgtcatttttagaccctttactaggcatttatagatgtataaatgtgtgtccatagacaatatacagtgctgtgtcatgctagattttgatctacccatagcagaagtgctgaattttgattttaagtttctgtgtccccagttctcagccaattaggaaacaatcaaatacaccaaacagacctttgtttttgagaccctgaaaccttagagctggaagggccgttagtaattatggccatctcctcctctttgctggaaggagaaactgaggttccgaatggtgcactgctgttctctgagtctcagagcagtcagtggcagagctaggggtagacctgggattctggctttttgtcctgctttaaatatcctttcctccatgctctggggcaggctaactccccggttgcctcccaaggctgggtgtggagcttttccatgcctcaggccctcccctgcctccttccctgcag

SEQ ID NO: 6 - DNA, 2141 bp, Homo sapiens
gtaggccatgccacttccatttccagtagagattttactgagggacactgttagggtgagggtagagttggtggccagggtcattctttccaggtgtggtgtcacaggcagtacactgttgcggggttgaaatttgttgccatactatctgcttgctctctgattctgatgtcaaaagcaaaagagcagtcatctttttgaaggtacctgggcatattcctatgattgtagacctggagtctcaggccacagcttctccttctgcccaagggacaaaataatgtcatctattttctgttctttgaggctactcttccctgtggattttaagggaaagagtaaggcttagtgatggggaagctgagaggccccagggcaggtgggtggtgggcctgtagggtgaggtgttactttcacactcaagtcagaacaggtgtgctggggttttgaccttctgcagcaaaatttccctcctcagaaacttagtatggtgttcggtttcaggattaatagaacaaaatgccagctgcacagcatgtgttcctgtaatatttttcattatatggctttgattatccttttgtgaatctctcacaactttaagttgttagttcttagatgttttctcagtacctttggcttgaaggagtgatactcatcttttgtttttgtttgagacagggtctcactctcacccaggctggtgtgcagtggcatgatctcagctcactgcaacctccatctcccaggttcaagtgattcttgtgcctcagcctcctgagtaactgggaatagaggtgcgtgccaccacacccggctaatttttttttttttgagacagagtctcgctctttcggccaggccagagtgcgtgttgcaatctcaactcactgcaacctccacctcccaggttcaagcgattctcctgccttagcctccctgagtagctggaccggcacactccaccatgcccggctaatttttgtatttttagtagagacagggtttctccatgttggccaggctggtctcaaaactcctgacctcagtaatccacccaccccggcctccaaaagtgctgggattacagatgtgagccaccacgctcggcctttttttttttttttttttttttgagatggagtctttctctatcacccaggctagagtgctgaggtgtgatctcggatcactgcagcctctgcctcctgggttcaagtgattctcctgcctcagcctcccaagtagctgggattacaggtacctgccaccatgcccggctgatttttgtatttttagtagagacggggtttcaccatcttggccaggctggtctcgaactcctgaccttgtgatccacctgccttggcctcccaaagtgctgggattacaggtgtgagtcaccgcacccagccctattttaatttttttaaagagagagataggggccaggcacggtggctctcgcctgtaatcccagcactttgggaggccaaggtgggtggatcacctgaggtcgggagttcgagaccatcctgaccaacatggagaaactctgtctctactaaaaatacaaaattagctgagcgtggtggcgcgcgcctgtaatcccagctacttgagaggctgaggcaggagaatcacttgaacccaggaggcggaggttgcggtgaacggagattgcgccattgcactccagcctgggtaacgagagaaactgtctcaaaaaaaaaaaaagagaaagagagataggatctcgctctgtcatctaggctagagtgcagtggcatgatcatagatcactgtagccttgaactcctgggcacaagtgatcctcttgcctcagcctcccgagtaactgcgactacaggtacatgctaccacaccccgctaatttttaaattttttatagatgtgggctctcactttgttgcccagactgttatggaactcctgggctcaagggatcctcccagcttggcctcccacagtgctgagattatagatgtgagcctgtaattatagacagcttggcctatttacctgttggaaatgaagaattatgaattttacatttcttcaagaaaaggttatgggagagttactgactttttttccttggattttttctttttaaatag

SEQ ID NO: 7 - DNA, 3210 bp, Homo sapiens
gtaagttttgttggggatgaaagacaactggggtgttttccttgagggagagaggggtaaagatccttcttaatccccagaattagaaacatcaacctgttctttcagctgtagttattccaaaaagtcacttcaggccaaagtgacatgaacagaagttccatgtgccatggagctctctggcttggaacatttccgtgaatatctgggagttggctcctccttaaggagaagtggaaagtcccttgctgagttgttctccacacccatgtggtataaagcagctttccaccttgcctggggctttccaaattccccatccagctcctgcggctgaccctgcttggctccatttttagtgccctgtttttctctcccactgaggtgggatagagggtgtaaaagcaacagatttgagttaaactttaaaataaatgaccaccttgcattagcttgcttaggaaaagagtacataaaataaaatgaacaaacaaaaacccatcttgttctttatcccccttattttctgcttttcattgattcagattattggattcttattgtcaagaataaactttaaacaaacaaacaaaaaaaggtaaatgtgacggaaggctagttttcagtcatttttaaaaattgtgatgccccgttctttttcttacatttgtcccctgaacaattcttcctctttaaaatgtagcagtcctagctgggcgtgctggctcacaccccgtactttgggatgccaaggcaggctggtcacttgaggtcaggagttcaagaccagcctggccaacatggtgaaagcccgtctctactaaagatacaaaaattagctgggtgtggtggtgcacgcctgtagtcccagttactggggaggctgaggcatgagaatcgcttgaacctgagaggtggagcttgcagtgagccaagattttgccactgcactctagcctgggcaacagagtgaaactctgtctcaaaaaaataaataaaataaaatgtagcagtcctttttaaaaatgtggaattttacttgacagtagagtgaagtagcctgtatgcaatgatatgggaaaatgtacatgacatattaagaaaaagcaaaatgtaaaataatttgaatagtattattagtatatgtgttttaaaaatacactatactcttatgtgtattcatatgtatattaagaaattctggaggaatataccagcagtgctatgtgtattagtgctgctgttggtatccatggctattctagactgtctctgtgatatttgcattttaaactgaatatattacttttataatcagaaaaatagtattaaaaatgaattataatttaatttcttttttcttttttttttttgagtcggagtctcgttctatcggattgcagtggtgcgatctcagctcactgcaacctctgcctcctaggttcaagcgattctcctgtctcagcctcccaagtagctgggacgataggtgcatgccaccacgcctggctaatttttgcatttttagtagagacagggtttcaccatattggtcaggctggtcttgaactcctgacctcgtgatccacccatctcggcctccgaaagtgctgggattacaggcatgagccgctgtgcccagactagaattcaatttttgagaattcattgacaactcttacttaaaataaggttgctgtactgatgtgagacattgttgtagtcagtttggaaaacaatttggcagtataaaaatgaacatacctgtaaaccaacggtgccattcccaggatttaatagcagagaaatctttgcatatatgtcccaggagacatatataaagtggacatcagcctgattataagctctaaatgcaacccaaataaatacccatcaacattagaatgaatacattatttgtggtatagacacaatggaatactccgcagctgtgaaaaggaatacactgcagatacacataaccatgtggattcatttcacatcaagtgaaaagtgaatcccaaaagaattcattggagtccataagtgtaaggttcacaaatgtcccaaactaaacaatacctgcattgcttagataaacaaatatggtaaaactgtaaaaaaacaaaacaaaacaagacaaaaagggctaggaaatgataaacccaaaagacaaaatagcagttatttctgagggaggagggaaggggatggggttggggaagggcacccagagaattttaggagtgatggacttttccttaaattgaatggtgggttcatattgttttgttattctttgtgccttacgtattttacaaataaccaattggatctatgtaatattataatacaaactgagtaaaggattaggttgaggatcacagcattggaagttcttggtgttgaagagagtaagtgccgagcaagttgtgtccctggcagtttgtttgtgaccacctggtggcttacccttcttggtgtggtgaggcttggcatgtcactttccttggctgtggctgttagtactgaatgccattctctctgaggaaaagtgtccttctcttttttattgattgactgattgattgagacagagtctcactctgtcacccaggctggagtgcagtggcgtgatctcggctcactgcatcctctgcctcctgggttcaagcgattctcctgcctcagcctcctgagtagctgggactacaggcgcccactaccacacccagctaatttttgtattcttagtagaaacggggtttcaccaaattattggccaggctggtctcgaactcctgaccatgtgatccacctgcctcggcctcccaaaatgctgggattgtaggtgtgagccatcacgctcagcctttttttttatttaatttaatttttttttaagacagggtctcactctgtcaccccagctagagtgcagtggcacaatcatagctcgctgcagcctccatctcctaggctcaagccatcctcccacctcagcctctcgagtagctggggctataggtgtgcaccaccacacccagctaatttttgtattttttgcagagatggagttttgctgtgctgcttagactggtctcgaactcctgggctcaggcaatcctcctgccttggcctcccaaagtgctgggattacaggcatgagccaccacacctggcctaagagtgtccttctcgttactgtag

SEQ ID NO: 8 - DNA, 3385 bp, Homo sapiens
gtaaaggatgaaagaagcttttcctttctttctcgaaagctagattgaattctgatcttaactgcaggcccacagaattggtactatatctccaacgtggggacttttccatattcaaatttagcccaagaattaaagtttttactttatttcggccaggcgctgtggctcacacctgtaatcccagcactttgggagaccaagatgggcggatcacttgaggtcaggagtttgagaccagcctggccaacatggtgaaaacacatctctactaaaaacataaaaaaattagccgggcgtggtggtgcgcacctgtagtcccagctactctgggcggctgaggcaggagaatcacttgaacctgggatatggaagttgcagtgagcggagatcttactaccgcacaccaaccagcctgggagacagagtgagactccatctcaaaaaaataaaaataaaataaagtttttactttatttggagaaactttgttttaaaaaatgtatttatattattatattttaagtatattttacttaataattcaattaaggcttttggtttaactgtatttaacagatagacaaaccttttaattttagttattttagtaatctaaaatgacacatgccctttttaagggaaaaaattcaaatacagaaaattaatcaagagaagaaaaaatttttaaatgaaatcatcagcagtactagtagttaaaatttagttgatgctcaatctagacatctgtcattatgtatatacacattatgtatatacacataaagatagaaatttatacagtttatattaggatcatttttttttctttttttggagtcagggtctcactgtgttacccagtctagagtacagttatgcagtcatggctcactggagccttgacctcctgggctcaggcagtcttcccaccttagccttctcagtagctgggactacaggcatgcaccaccacacctggctaatttttaaattttttatagagacagggtcttactttgttgcctgggctggtctcaaattcctgggctcaagggatcatcccacttcggcctctcaaaagctctggaattatagatgtgagctgccgtgcccagcccaggatcttcctttatatgcttttctgtaatttgcacttttaccttcatccagcatatcttactgcaacccttcctgtgcaaggccctatagtgagcatgttgcaccagcttgccttaggagaaacttgagatacagagcctgcactggaaatttagcgcaactctacatgagaatgcctgtctattcatatcctcactaaccctgagtgttgttaatttactgaaagcagttttaaatgcttcctgaccagggaacgaagaagcttaagttctgggaatgggaggatagaagtgccagaaaagagctcaggagttcagaaatccctgcagcggtccccctccctctcctttcactttctgtctttctggtcttttggtctttgttacactagtgataaaccatcaaagaatgatggaatgatgctaacttctctctttttttaatttttttgagacagagtctcactctgtcacccaggttagagtgcagtggcatgatcttggcttactgcaacctcctcctcccaggttcaagcgattcttagtcacaaccttccaagtagctgggattacaggcccatgccaccatgcctggctatttttttgtattttagtagatcgacctgcctcggcctctcaaatttttgggattacaggtgtcagccactgcacctggcctaatatctctattcttggagatagatttaatgagctttttctccctctctattcacttattccttgtgcatgttatcaatattttgaaacataatgtcatgtcctttgatcagttgaaggctgacattgaaaaggcttatggggattgggtgttgtggctcacgcctgtaaatcccaatgctttgggaggcagagtcgggaggatcacttgaacccaggagtttgagaccagcctgggcaacaaagtgagatcccatccctacaaaaatttaaaaaactagacatgtgccattacacttcagcctgggtgacagagtgagactccatctcaaaaaactaaactaaactaaacaggcatggtggcacacacctatagttctagctactcaggaagctgaggtaggaggatcactcatgtccaggagttggaggaggcagtgagctatgatcatgccattgcactgcactaggccacagagtgggaccctgtctcaaaaaaaaaaaaagaaagaaagaaaagaaagggctcatgtagttcaagcccttctcttcatgcaaggggatgctaaggcccatgatggtgaagggcctggcaaagcttgcacagatagtgtgtgacagagctggctcaaacccatctttgggagctgtctaatctctttttctgagtctttatgttcatagacaagttaggatgagtaaagtaagtgctaaattccatatttcgtgttctgcatatctgggctcagatgcttgtcattttccagtgataactccatcaatgcctcctagtggtataaattttaatacttcttgtgtgcccagccccctcttagaaatttgagattttaggaagggactagtaataaaaggtaaaataaattattttctggccaggcatggtggctcacacctgtaatgccagtacttcgggaggtcgaggcagatggatcacctgaggtcaggagttcaagaccagcctggccaacaaggcaaaatcccatctctactaaaaatgcaaaaattatccgggagtggtggtgggtgcctgtaatcccagctacttgggaggctgaggcaggagaatcacttgaacttgggaggcggaggttgcagtgagctgagactgtgccactgcactccagcctgggcaacagagtaagactctatctcaaaaaaaaaaaaaaaaaaaaaaaggccaggcgcagtggcttacacctgtaatctctcaggaggctcaggcaggagaatcacttgaacccgggaaatggaggttgcagtgagccgagattgcaccactgcactccagctcaaaaaataaataaataaataaattattttctttttttatttattttttcagcatccacccaacatggtgaaaaattcctcttttcttaatgtcactgaactgtaaacttaagatgaaaaattgtaaatttcatgctatatatatttcaccacaataaaaaaattccttgttcttattgtagtggtctccatgtcttcagtatttccttccccttctccatctcacctgtatacattcactttggtaattagcatctttcttaatttattggcag

SEQ ID NO: 9 - DNA, 2394 bp, Homo sapiens
gtaggacctgcctcctgataactaatgtgtctaatgccaccagagcgtctcccaggcccaggggctttcctgtttatggaagagcttattctgagagctgtagaagcctattggattccctgtcacgcacgagaacaatccatgcatgacataattctaacctgcccgctgcctcaccccacttttgggagcagatggaattgcatcagaagaatgagacacttcgagccctgaggtttcttcccccctgcaactctctggacctttgaagaaattaagatccagattttaagagtggctctcaggggtgtttgtggtagtgtccatggtgactaatttctcatgaactttggaattatttactataattctactgactttaacagtggagttcctgttaagtaaaatctggaaatctcagtatgtcaaacataaaagagctgctggaggtgaagtagggctggggacccagcaacccacccgactctgcaggttttatctcatttaccctcccttcggcccgtccctccgtaattcctgtggcgctcatcagatgggactggtttgaaaacccctgtctaaaaacactccgggccaggtgcagtggctcacacctgtaatcccagcactttgggaagctgaggtgtgtggatcacttgaggtcaggagttggagaccagtctagccaacatggtgaaaccctatctctactaaaagtacaaaaattggccgggcatggtggtgcacgcctgtagtcccagctacttgggagtctgagacatgagaatcgcttgaagctgggaggcagaggttgcagtgagcagagattgtgccactgcactccagcctgggcaacagagtgagactctgtctcaaaaaataaaaataacaaagctctctaggcccagtccaagcccagccacagcctccgtctattcttgaatctagtccctctcctccatgaattcattctgtacacgagggaaaaggaggagttcctttgttgtctctgtccctgactcgttggtttaggcctggcttttgtggcactctgtaattactccctgcccgaccagcccccagacaaaatgtcttgttaccgttctgtctgcaccaccccctgccagtctcccggacacctccacacataccttgttctctgtcccttcatacccctgcatagtgtgactcacacgatgagcaggatcccctgacttttttttaaccccctagagaaacataccaaactgttagtgagaattgacctctgaagctgtgtgcttgggtaactgcctgcctgttgcagtgatgccttgacaaagctgtgaaagacaccaaattgctcatacagcatgatgggatcagagttgattttctttgcatatttttttatatcaagcacatcatagataaaaataatgccttgttattaatatattagtttaaaatggaatgctgatttttggctgggtgcagtggctaacgcctgtaattccaacactttaggaggactttaggaggctgaggtgggcagatcccttgaggccaggagtttgagaccagcctgggcaacatggcaaagcccatctctataaaaaaaaaatataaaaattagccaggcatgatggtgcatgcctatagtcccagctgagtttggaggatcatttgagcccacgaggtggaggttgcagtgagctgagatcgtgacactgcattcctgccttggcaacagaatgagactctgtcttaaaaaataaaaattaaaaaattaaaaagtaatactgatttttattggattactttgagccagttctcagtacgtcagttttgttactttatagtctcatcaaaaattcattttgatccatcattagacgctttgctaaatgatttcggactacctgcagaaatcatgcctcccatcagaggatgaagatttgccatctttgcagttagccaagagtgagcagcagagcctgcctttcaagggctcagcctgcttgagtgaacagtgagctttgcttatttattgcctatgaaagccatgttcctgggtagcagtggtctacatagcacatacctgacagttctcttgggtgtaaagtcactttgggctccctgggtagactattaaccttttgttacaaaagcattgtctgagagtgccatctctgttgcattgttgggtgaatgcctgataaccatcaaagcaagcagaaggcagttttttgtctccccactgatttttgtcctccactttttttgcttataaaacaaacatcacagaattgattttgatattacacagttccaaagtcagattccatggtaggggagcaaaggctatgcagataagagtggccccaacccctcattctcccctctccctcgtag

SEQ ID NO: 10 - DNA, 2837 bp, Homo sapiens
gtatgtgccacctccttggtttccatggttccttccaccgtgcttcattttaaaaattttctacaaatgctggagccttaggaaaaaaaaaatctgttaaagcttttctttctgtctgcacatgctagtaggttgtgattcctgactgtgagggttggggaatttttaggcatggagccagtgtcttgctgtttctcggcttatttttttctttttctttttctttttttttttttttttttttttgagatggagtcttgctctgtcacccaggctggagtgcagtggcatgatttcagcttactgcaacctctgcctcccaggttcaagcaattcttctgtctcagcctcccaagtagctgggactacaggtgtgcaccaccacacctggctaatttttgcatttttgggagagacgggggtttcaccatattggtcaggctggtctcgaactcctgacctcactcaggtgatctgcccgcctctgcctcccaaagtgccggcattataggtgtgagccaccccgcctgatcctgccagctccttggcttaaaaccacataagttggctcctaagtcagtgaagtaaggaagtaaggtgataccacaagaccttgtccctaaaggggcccatcactagttgttggctgacagcttgctgtgaccaaatgcaggatacagtgtgataggatggtatctcttgataaggggagggatagcaggtgtgaatatagcacctttatctctgtgactagaaatcagtcttcagcaaggatcaaggacagtttttacctgtaggctttggaatagtctggaggctctcatttttacctggctgtcaatccctataggaggagttttcttatgaaaaattagagaattgtgaaactccttcctttaagaagaaaatccagtgcctttgtagctctgtgttacctccagcacatggcgcgatggacagagtgggtgaggttggcaaaggagcttcaggcagaatcactcagcggtgctaaactcacagagaactgccagtcggagctgagtcaccatctgtgagtcacaggcctagcagtggaaccccagcccttcccttccccttgcacctcccctccccctcaggaagaaaggatccttgaggcctgacttcgttgggcaaaagaatgcctgagttggctgcttgctgtgtgggctctcaagcactgtggggtttctcactggctttatggggaaccctgagggttctcacaggatgccaagaattcccttcatccagtaagagtggtttgcttagtcaatgggtggaactttctacttggatattaatgaactttaagcaacaaaagaccccagatgctagaagattgttgggagttggacattttgaaattgaaatgaatggaactttctcttgtgtgtcttaatccttttcaaaaaacatgaaatgatggacttgctcagaacctttcatctctctctctccctgtctcatcaacttttttagagcagttttaggttcacagcagaagcaggcggaaggtgcagaggtacataaacccatgagtacccactgcctctgcacatgcgcagcccccccattgtcagcatccccactagagtggtcaacatgcgtgaacacgttatcatcaccaaaagtctgtagactccaagcctcttgttttcatcttgtctttgcctgatccagtgtagggtggatatgtagggagccagggcagctttcaagaaggaagtatgacctgttgatagcgcacactgcagtgcagcagggaacactgctgggtcctttgctagaaatatcttgttggtttctctgcaacatcttccttgacaccatctgttcttttgcctccaccaaagattgcccttgttacaggtataggtttcaccagatggctggtttcagtatctgcaagattacttggttcagttcttacatttattaaactgcgcattttattatctttattccttccccccacatccatcctgtttgccagtttccagttgggcatagaacccctaaaatagcatttagcattgtccaaactgaaacaaatgagctagatactcctcctgagccagttaaattaccctaattttatgcttaacatcaatatagtacctgtctgggcatggtggctcatgcctgtaatcccagcattttgggaggccgaggtgggcagatcacctgagatcaggagttcaagaccagcctggccaacatggtgaaaccccatctttactaaaaataaaaaaactagccgggcgtggtggcaggctgctgtagtcccagttgctggggagcctgatgcaggagaatcgcttgaacccaggaggtggagattgcagtgagccaagatcacgccatacactctagcctgggggatagagcgagattctgtctcaaaagaaaaaaaaagtaccagaacgtcttcaatgccctgtgtggctcgcattggatttctcttgggcagtgctgctggagtaatccacagggcaccaatgcgagcttgtgagggatgcatatttgcctgggtcagaatctacattttaacaagacctccaggtggcatgtaggctctgctgtagaacactttctttctgctagaggaattagtcatctttgcagtaagttctgcagtacaggatcataaacatgtgaaaggcatgaagaacagcccagagcgtttaattcacacagctggtaggaggaagaaacagctccctaatgacaaatgcatctgtgggcgggaggggacttgggattgtgctggaactggatttctgaattgctactacatcttttttcctctcatccag

SEQ ID NO: 11 - DNA, 3662 bp, Homo sapiens
gtaatgattggggaagggctgagcaacgggcaggcggcgcagggcttttctgggcttgtggggctcttttccctccgagggaggcggcgcgcccgcaggcccagcgtggcggggtcgggggcacgtgcaagccgcctccctgggtgggaactggcgcccccgctcaccccgaggcccggggcgggggcgcaatgccaggcgcacgtgctgggcccccgatttctttggaggggaaagtgcagatcaacacgtgattaccgactgcggggccgagtcactgtcagggaaggtcgggagctgcacacgcgagctgcttagcacagcgggagttgggagtggaagttgcttttccatcgtagccaagtgtaaggtttttgggatagacgcagacttagcggcggtggccactcaggccaggcccttcctaaaatggaggagggcggcgatagtggagatggccacgaggctgcggccccctcccccaccggtagcccccgcctctcgcccgcgctcgcggcccgcgcttctgcctagtaagcggtgactcatttaactccgagaaggcacctcttgaggaacctacagcagagccgttagatcattggcaagtcctccaggcgacctcgcccaagttgggcaagagcggctccccggctggccaccaaaaatcgggcctgatctgatttggtttttttttttttttgagttttgctattgttgcccaggctggagtgcaattgcgcgatctcggatctcggttcaccgcaacctcggcctcctgggttcaagcgattctcctacctcagcctcccgagtagctgggattacaggcatgcgccaccacgcccggctaattttgttttttttttgttgttgttgttgttttagtagaggcggtgtttcaccatgttggccaggctggtcttgagctcccgacctcaggtgatccgcccgcctcggcctcccaaagtgctgggattacaggcgtgagccaccgcgctaggccaagaaggatgtattttaagaaagcttcctgttcaatgttcccctgctctgtgcgcttctctgggcggtagggtccattgcttcacttgcctttcaggcccacccgggccctgactgctccattcatttaccgtgtccctaattcggggttccatccaggcagctaaaaaagctgtgaatggtccgggcgcggtggctcacgcctgtaatccgagcactttgggatgccgaggtgggcctcctcaggatcacctgaggtcgggagttagagaccagcctgaccaacgtggtgaaaccccgtctctactgaaaatacaaaaatcgaccaggcgcggtggctcacgcctgtaatcctagcactttgggaggccgaggtgggtggctcacgaggtcaggagatcgagaccatcctactctactaaaccccatctctactaaaaatacaaaaaattagccgggtgtggtggtgggcgcctgtagtcccagctactctggaggcggagcctgcagtgagtcgagatcgtgtcactgcactctagcctgggcgaaagagcgcgactccgtctcaaaaaaaaaaaaaaatcacctgggcgtaatggcaggcgcctgtaatcccacctactcgggaggctgagacaggaggctgaggttgcagtgagccaaaatggcgccattgcactccagtctgggcgacaacagcgaaactccgtctcaaaaaataaaaaaaaaatcacactttattttcacatcctaagacgtttgtctttttttttttttctgagacggagtctctctgtcgcccaggctggagtgtagtggcgcgatctcgactcactgcaagctccacctcccgggttcacaccattcttctgcctcagcctcctctcctgacctcgtgatccaccctcctcggcctcccaaagtgctgggattacaggcgtgagccaccgcgcccggccaagactttctttcgtcttaagtcactaaaatgaaaacaaaagtactgaggcaccctaataaaattgcctttaaaagtatgactgaaatactcaaagtataccacagtggtggcctggagatggggcagggggatattgactggaaatggcctgagataactttttgcagtgatggaaatgttactcgggtatataatttgccaaaacgcaaaattaaatctaaaaatatgtttaaacaaatttgcctttttggaacctagtaaaaagtaagtacctttttttggctaatgaataaaactccattgttacatagacaacaaagccacagaagtgacttcagtaaataattttgaaagttaaaaacctgccacttgccacctcaggacattccagagaagatgttggaggggagcccaggtagataatggctttaaacactaagttcctctcacccacctcagttctataagttgttcagttagtttctttggagagcgccaaggaaaaaagtaagaatgggtttttctttgaggaattggtatttgttgttcattgctggaaattcttgtatttttttttttaaggtggtgttaaaggaagccccttcccttgaagtatcctggtgttcaaagaatctttcctctatttacaaattataaaaccccgttgggaagaaataattagatgtcattattggctggtcctggtggcttacccctataatcatcctagcatgctttgggaggccaaggcaggggaggatcgcttgagcccaggagtttgagagcagcctgggcagcatagtgagaccgccatctcttaaaaaagtaactaaaataaaataaagatgttatcattaatgacataacaacactgaagacccaagtactgtgaaattggaaagtcccaggaagtggcagtagccaagatcacacggtccaaggacttcattgttcctgacttccaaactcagaatttctgatcaaatacctactaagtgaatattaatctgaagaattatgtccaaacttcattcactattttgttttaattctgctacaagcaaggttttcccctggtcccgccaccctctttttggccctaggtggttttgcttgtgaccttcatgtctgttcttggttcctttccattcccatgccagccctcattgttcactcatgacaatagccagtttcagacagctcctgggccactctgcgtcgagcaacacattcatctttctatttggatttgattctccctgggcacacacgtttgtgggctttttctctctctttttttttttttcttttttcttacagaacaagacctcaattgctagggtttaacccatttatgccagaggttgcaaatctttttttgtgaaaaatcagaccttggcaatgacttgagcagtcggataaaagtaactcccacgcccttagcgttccagtaatggaatactaggcataaaaaggttaaggccttccgcagctaggccctaactacttaattaggagaattctcactgctttccagcaggcttttccccccaccctagccagctttcctacatccagtatttagtccagtgctgattctctctagtccacgaacctcggaagtgattccttgaatgagtgaggaaatgcagcattatggcttatggaaaacatgaactgtgtgtttcttctacag

SEQ ID NO: 12 - DNA, 2366 bp, Homo sapiens
gtagggccctaagggcaggtaacactgttaggataaccagcctcttgctccacctgctctaggagaagacagccaggcccaacctggcatctgggcacagagcctcttctcgtctgtaggaacaccgccagggaggtcatggcagggcaggaccaaagggtcctgtggctcagtaggcacagtagatgtcacaggcacttggtgaaggactggtttctgtggagtcttgatcttggctcagctcagaatctccagtgattgggctcctcttggcctttgttcccaggaacatgttcctcaccagctgtccggtgactcttcccctccctctccttttgtgacaaagctctgacaaagctctgtccccctctcgtccctctggacggatgttgctcccctagattgcccgtgaggcagaggctgccatctaccacttgcaattatttgaggaactccgccgcctggcgcccattaccagcgaccccacagaagccaccgccgtgggtgccgtggaggcctccttcaagtgctgcagtggggccataatcgtcctcaccaagtctggcaggtaggaggcggcagcggctccctggaatgccctgctcagtggtacctcaccttgggggtcctgggagcagtccattgaacaatgctcaggtggcactgagccaaggtaagacccctctgcctgccaccttgggcctgcagggaaggattgagcagagccccttccctgggcccaaaggactctaggtagcactcataaggaatgtcagaacatttggatcaaaagcaaatttatgctggagatttattacataacagtgcacaggctgactacaaatggttatttgatattgaaaatttagtcctctaaaattgtaaaagataaccacttttgcttattccagttactatgtgctctttaaaaatttcagttgggaaatgaatttatttaaatgctgtttactgtgcctccatttggcacactagtccctgctgtttttgagccctaaagacaaattgggttccagctcaggagaggttgctgtgctatcttggctgacattctgtggggcctggcagccaggctgaggactgtgtggcctatgctgggcctccaacttgggatcccttccttggcccaggacattgagttaatgtccttcactctcctagttagggagtatgctccttgtccctgtccacagggcagcaagggtttcctggaagaggggagcaaacaggcagtgcccatgcactgaggagcagcagatgggcgtgggcagcccagagaaccaggacacaagctctgtgcagatgccctcagcagagggctccagcctcccactcttggctgaacagctccaacccgtagggttgacctttcttaaaaggtccagttcttgctgtttggctattttaagctctagtcttctggggtttcactcagctggtcctggcttcagcaattgcttccctctgaaggccttgcatagaggccaagcgtgaagtgcagggacttctctgctgtgatgtggcttaagtttccctgacacctgttgagtgtcctcataacttcccttctggtgcccctccccagctcctgagacacagctgcagctacaagtgtgcagtgtcagtgttcaagaaagtgcctggcagaggggctttagaagggtcccctgccttccaaaggagctttggcaggcagagctgctcctgcagcaacactcccatttcctgttcttgcctgctgagtagcacctagatttctaagcctcatctagatactcagatttgattctgggcctttatagcccagttgctgggactgtttcaggagctaggggccatgtggggcagggagagggcacaaaagtagagaagcctgatgttgattcccagggggctggtcagctctgctactgctccttgcagatgtcaagagtcaggtgctagtcacgtgctgcttggcttgtcactgtcattggcagcgagaggaatgggtgctggtgacattgggccagggctgcctctctgtgtcagagttcagggtgtaggaggggttctgccaaccatgggctgtgtggggtaagtgggtgaggctgatcttgctgggtcaaggtgatcctgagcccttggcctgtggaatgggggtagagggcaaatggtaacctagcatgctgtgggggatataggatgaggggctgcccgagcctcgggaggggtcctagggagcagatgttgaagaggccagagccctcagtgagctggatgagagggtgagctgtttgaacgccctgagggtacttcctggggcctcgtgtaatggtctcttctgtatgtcccccatcccatctcag

SEQ ID NO: 13 - DNA, 3539 bp, Homo sapiens
gtgagttctggggcctgccccatcccccagggcttcggactgggcctgggatggatgcaagctctggtgcagagctttttaggtttctccatcctcttatgcacagcctttcattatcctccaagttacagcagcaagagggtgggggtggaagtggaggtggcttttttttttctcctgttctgcattcctgcccacacccccacccctccatttccttctgctctggaggcatcctccttcattggacaccacacagtttatttcacttctgacttcaaggttgtgaattcttcccatggcttaagtcctgggatacttctgcagtgaaaggaggtcttgtacctcttcctcagagtcagaagttctgagtacctttgccctattctgaaaagggctaggggctcctgctcccagctgccctcttcctttggcttccaattcagttccctctgccccgcatcctgcagacaggcgctcccgcagggggcccttgtggacctgcactggagtctgttgccttcactgagctgcctgtgctggccttgcatggtgcctgtagggggatttgctttgctgtgccattggggtacagctgctgctcttactctagaccaaaaagtcgggttgagtgactggtggcagggccacagatagagacagcggggagggtggctgaccctggcggccctggactgagcgtctggaggagtcgtggaggctctttcccttctttctcctctgagagctcgttcttcaggctcttccagcttgtcatgtcgagtgcctggccactgctcagggttggaggctcagtccctttgccctgtctgttccagctctggagctaactcagggatccctgatcagggttacataggtttggtaaaatgagtgctggaaattaactttctcccagtagtcttaggtcatgctcagtgaacttaaactttatccagatatggttttccttcagcctttctattccctttctagccagtgaaagacccgctgccctttgacctcagcccctccaagcccccaagtttaaaacgccaccccctgccaccagaaaaaacagaaaaaaaaaaaaaaaaaaaaaactaaaacacccatctggtctgggcatcttcctttcctttttcactatgtatcctgttactgggcttaaacagctttcagagaagagatgtcatttctattaaatgctctttcagtagcgaactgagttcacacttgactaaggatattttccggactgtctgtcatcagcatccttagtgggtttccccatatttaaattggtagaggccagggatggtggctcacacctgtaatctcagtactttgggaggccaaggtaggtggattgcttgagctcagaagaccagcctgggcaacctggtgaaaccctgtctctactaaaaattcaagttagctagctgggcatggtgatgcacttctgtagtcccagctacttggagagggggtgatgctggggcagcaggatcgcttgaacccaggaggttgaggttgcagtgagccaagatggtaccagcctaggtgacaaagtgacaccctgtctcaaaaaagaaaccaaacaaacataaaaaaaaaaacaaaaaaatcggtagagagtgatttctctcccaggcccacttaatgtagactgggcctggctgacacctcaccattcgtgtgatgtgattgctgttctgatgcttagatactcttggcgcagtctcacaattgccaccatggtaggaaggtgtcccaggagacggtgcaccttgaaccagtcaccactaaagtggctgcctttctgggtctctccacacatcccctctctctaatttccctacttaatcgtgtgacttcatggtctcaaaggaggaacagaggctgatcttgacttagatatactgaaccatgaaatcactgcatagaatgtggggacttgaatgtgtctttgggcaagtcatttaacctcttaagacctcatctgtaaaatggattagatatgtttaattatagccttagcattaaatattcattgctgttattattaagtgtctgataagtctctgtgtacatggatgtaatcttcctaactcccattacctccatttatagatgagggttatatggccaataaagcctgggtttgaatctaggtctactgcctccaaagccagtcttctctcctgcaacatcatgctctgtctagcaggagatgagaacaggtctccatttggagcctgtcagtggggtcagagactaagattcaggctcagggtctaaattccatatcctttcttccataccctggtgtttcctatgaacagatagatactttagggctgcaaggtttggattgcatggcactgctcagaagataagttacaggtctgggctaggctgtagctgcccctccaggtggctagacctttcctttctgtgtcaccagttaacactggccaacagttccttccattaactgttcactgctttctcctgtgtctaactgatgcagtttatgacccataactaagagcagtaccaggtatggctctgtttcctgttcatgtcccctgtcctctgggctgcatgcattccgttcttacagaaagaatacctttaacctagtacatcctgccacacatctgcttctactgtgaaattgatgagggggtattaccgattcttccctcacccatcatttactgagatgctggtgattgcattataatcctctaaagcttacattgtctttctgattcttggtcttatctgagcaagtgatctataaataactcagtggctttctcatgactgttttaattattagattttaatcaagtgtcttattaaatatatctgcatgcttccacaggcatctgtctcttcacatggctgttcagtgtgcctctcacaagttagcccacgttttctgttctcctgcttcaaactcagttgagctgccttgctttggctttgatcccagctttccagcgctgctcaatctgttgccatggcaggccattggaaaggctcagtgcatccccgtgcctgaagccaagtgagcgctcactccatgcatgcatggaggctgggcaggagcctgcctaatcaaccagccatgtgaggagggagggcctgttccttcctgtaagctatgtcatgaggcagcgtggtcaagtcctctgccagggagtggcctgggcccagcctgggcatgttttcatgccagggtgctagagcctactgccagattgtctccctccacccccaatgaaaaaatccttccagaagggaagagccaatttcccctgtattggaggggaagtggcagcacctcctgaagcagttggactttcatcaccctacctctgcatctgcctgaaggacagatttagccaattaacctaaggttaccttcctctctgataaattccccattctgtcttcccatgtgttgtgtctcgtttttttcctcctccttccctcttccttgccccctcttcccctaaaccttacag

SEQ ID NO: 14 - DNA, 2728 bp, Homo sapiens
gtgagtgggcaggggctccctccccatcaatagggccgacccaagtcttcctcccccttcccccatgccgggccccacgatagtgtgaatgtcaggggcttcaggtttccctaaatataggtccctgccagaggatccgtggcgggaaaagggcaggggtcattagagaagatcggggacacatgtggggcgggcaggagctgccttataaccagcccgggaacccctagctcactcgctgctgaccaggctctgccggctccttcggcctcgccgcaggtgggccccttgcaggaccgggccggggtggggatggggttggggttgggccaacagggtccagatggggtccaggtgaggaggggagatttggacgataggagcagggggctcagcatctgggaggcagatcagttcggggacggattttcttttggagaaggaagtcaggctcaaggaagacgtttggcaggaactgtgaccccgcatgccagaggccgagcagcggccggtgcatagccgcgcattctggttttctgtggcgcagaggactaccagcctggctgcggcggcccggcggagagcgcgcacgcatgcgcgacccagcccggcctgccagcctggaactcggatggggaggtctcgcctccgcgcgccgctagttccgccgccttctcgcgctccctgcgtgtcctctcgatgccctttcctccgcctcccttacgcctgggccagtgacagggtgtcggctccgcgccgattcagcccgcgggcgaggcaggctaacgcacgactgcgcgatgtggcccctatggtgacacgcgctgcagccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctccttgaatccactcgccagcccgccgccctctgccgccgcaccctgcacacccgcccctctcctgtgccaggtgagcgcccctcttcacgtgcggggaccagggaccgtggagagggatcttgggggcagtggcgggttgggcgtccgcgtggaggcctcccccatcccatgccagcgtctccccactaccaggcacacacaggctccccggcccctccagcctgaggtcctctaactgcgcaatgcagcggctgcgcgcgctgagtcatggcgggggaggaagccggacgagatgaaggaccattctcccccttttcttgcagggacccctgtggcaaaggattagggccccttagccctggcggggatcctaagaggcagtgaggggtgggggccggcccatgtacagccccagggttctcgcaagtgggagcttggtttctgtcctgggaaacggggcgcccttcgcgaggagggaaacccctccgcggtgcttgatgcccccttaacactttccctgtctctccttatcgggcgaccttgattctgagcccggaacagctgcagccatgcgaagcgacgggagcatttttcaggggaaggcgcttgctcctccacgttcttgccccgtaggaacagtgacgatggcaaagcttaccgctttcctcgcctcgggctagggcttgttccgccgccctttcctgggcttctccttgctctcttatatttttcctaatgcccctttcctaccacccgccccctcccttgtggggaaaagcctgaccttgggatgtccttgaagccttggagcccgggccagccctgggatcttgaggggattggaaggaacaccccagtggcagtcagaagagctgggttctaatctcagatctggctccggggttgctgtgtggcttgaagcacagacctttcccatatctgggccctttcccacgagggtgttgggccctctgcttgattcacgatctttacattctaaaatactccggttcggttttgttttcaggcaaggtgaccccatggcaaggcgcaagccagaagggtccagcttcaacatgacccacctgtccatggctatggccttttcctttcccccagttgccagtgggcaactccaccctcagctgggcaacacccagcaccagacagagttaggaaaggtacaggggcaggcctagcaaagggaagttgggcgtaagagagagctggggaccagaagtgccccagggcctgctgggtgtggggcaggggaggtagggaacatttccctgacctccaggagaggggccctggtcatcgggagatgatgggaaaccctagctaactagtccttcccctctgtttcctgtatccag

SEQ ID NO: 15 - DNA, 2675 bp, Homo sapiens
gtgagtgcttgtgtagatatttgcacttctgattcaaggattgtaagaagaaaaggttagatatgttctcagcttttgttccttataaaaggttaagtccaaccaccagtttatttacttacgacaggagtagtagcatgcttaaaaatgtatacgtggaaatgtgttagagttgacttaattatcctagacatttcaaattggaattttgtcaacagctgtggctttctcaaatgttgagtcaaactcactgcattaaatatttatacctaatttcagatgaacagattattccatgattctaaagttgaacaaataatgttgctataaatacacagaaactttggcaagaagttttcaaaggcttttaggtgtacctgtagggacaaatctccataatcaagggtaaataagacagacctgcaaaacatagtaattgaagtgagtacttgaaaagtagcataaagagccttctgcttactttaaatcactttgaatgggtattgggttttttttttttttaaacctttggagagttttggctggcttggaaaatgaaaaaatttcactagtttaaggttaagtggggggaagtgtgacaaggttggcagctgctcattaaataatttttagattggcttccagtgatttgggaagagaaaactgttaacataggatgagtactgatgagtactgcatagtgaactttctccctaagtatcatttagggaaaatgtttgcacattcaggaattaagattattactaatgtaaaccactgccaagtgtatgttaatacttcaaatttagatatgctgtaggctacattgaatgtacttatcctcacagatacgaaatgagtaggaaaaaatatgtttgtgctatcagttttctcactaaggcttttgaaatattgtcaacttgtattaaaattagagaccataactacttttttgtactataatccagaaatcaaaagacctaaaagttaatatccatactcacatttaattagatactagcttttatggcagcatgtttcataggaagggctgtcataggttggtgctttgtagctcactgaagtgtcctacacagtatttgatgacaaggaggaagctatatcaatttctagtacacccaaacccaatcaggtagttcagggtcaaatgtaattatggaatcatttaaattctaaatgtaaataaatacaggactacttcagacaaaatgttgttttaaagtagatgttttataaaattggtattcttgtttggaagaacacagctcaactgttccttactccccttctgcatgttcaaaagaatgtgtagacttaggaacaagagacagcaaagtttctcggtcttgttgcctgttggcaacccttaagggttggggttggggcaggggtggagatgttgataaaaataaagagttgggattttaattatccataattctgctatcccctatgactgcaggtaattgatagttggctatatctgaaaggtatgaaaactatgtgacagtttagatgacagatgagatcaattatttgactcaactcacccagttgtactgattcacaaggtgccaccaatatgctttcatttttttggaagtggtttcaacatgtagttgtttcttgggactttggctgctcaggtcttagtggtgtatcagggagactgcagttctgggcagcttcacttaattggcctcagattcgttgattgtcctgtctcctctgcaatggaaatgcatgagtgatcctgtaatggatatagctgagatttggagtgaacatgggatttggagacccaatacctgggtttagaaagtctccttactaactgtgtgaccttgggcaagtcacctaactttaaagcctgttacattatctgtgtaatgggattaacaatactttcacatgtcctgtctcataatgagattgaatagatagtaagtgaaagaatgttgcaaaaaaatttcatagaaacgtgagatgttactcttcctcccttctcttaacagtctggagaaagtagaagcaacaagtctctaatacaagatttattgaattctggggaggtgataactaacaattccatgcagtcagatcatataaagaatgacatagttaatacacatgaattgcatttccactgatgtattatcagattggaagggattttggttgacatttgtaattgagtttgatacggtaacacaggggcaaagttggtttggtcagctttgaagcatgcaaaaccaaactcgcctgtcatctaaactgacgcagtgtggggccaccttctcctattaagtgcagtcctcaggggatagatggatatcctcatttgttaaaagttatatataatgcttttgcaaaaagcagtttttaaaatgatgtgtctgtcttgagtgttaaattacttgttttagttttgtaaatagtggtacaatgctggggccttgtatgattggagtgtttttgttgaagttggggatagtgtggtccccgctttcctaaatttattgatggtttggtcttatttaaagcaaatcttaagtgtatatccaagctaaaataatattgactctggtacattttaaatgaaaaagcacattattctcccctgttaatctatttttggaatgtgatattactag

SEQ ID NO: 16 - DNA, 3518 bp, Homo sapiens
gtatggtccacgtaaacatgtttctattaaaatttcctcaaacccgtgttaggactcagcaatatcatgcctctcctgcagactcatttttctgttaacacttcacgttttctttcatcagatgcctaagaggtgaacctattaaaaacagcttgtatagatgaggtctgctttatacataaatttttttagaaaatgatgagggtttggaatttcaaattttctgtccttgaacaggtaagataatattttaaaccacaccttttaaacctgcctccttggtagggagaaaggaaagcattaggagtatctgaattgtaccatggcattagtgaagaatcaacagtgttatcacttgaaatggcatacattatcttccactaaacacttctcagtttagatatgtacccccttttttttggagttgggagttgtcagctcaaagttgcaaaatgaaattgctccaccaagaatttacattctgcggggggaaatccagtctgtcgttagacaactatctacttgagtgggtattagaaatcttattaatagatgtcattattagagtaatcgaggaaaaattccatgaaccaagcatattttagtccatgccgtagtatactttgaggcagtgttcccagtcccacggtggtaggataggatggtgtaatagttgtttctctaagggtttgatatcaaagcattgagttttcataggtgatccccaaaagctgacaggtacctctgaagaatcaaaccaatacagtaataacgcatcttaactgatcacttaaatcgcaacttttaaatccccaaatttttaagtgtcaagtttattattctagaagtatccaacatatccaaaatatactgtgagtctataagagggggtttctgtatataggtttttaaattttaccaaagttggagcacttagtgaagttttaggaactagggaatctagtcgaggttgtattacatgtatttatcagcacatgtacattgagagttttgcaagagcagctgatgtcatcatattttttgtattaatatgtcttgtcctgagttttcagttaaaagagctgaggtcctaactgatcactaccacaattgaagatctctgattcagttttctaccaggagattgtgttagccagatgctggatgttgaagatagatgggtttttatttttcagagtaggtctaaaatagcctctcatggcagtccatctgttaagtccatgggttccagcagcagaatctttggtttaccttttgttacactgaagagcttaaatgttttgcctagtttcattgatgtccttaaaaatagcataattttgtgtttgtattcagtgcttcagttcaccgttgagtaaattgtttgtctttatattgacaaacaagcttgcggttgatttgtctcttagaaaggtatctttgtccattgttacccctccagtgcagaggtttttgccttttggtttaagttagacttaatgtctacgtgtttcaatgtttagaatgctcagtcctgccagatagttgcagaagtaacataaaattgcttggaagttatcaatgaatgctagactttccaaactgtgcaggaatcttttgttcaaatgagagtaaatgaaacatgctgcccctaatgcatggaatggtgtgaaattctgactggtgtgggttcaagagcaaactgaaagggaaatcttaagacagagtatagtacaagtgttgctgaaaatgagactaatagtagtaaggggaaaatggggggtcaaaaagggaggttttttgttgagaaataacatttataggctgaaatgcaatccaggataacagttgatggcaggaaagtgactgctggaactgttttctttctcagagagggtagggttttgcccacaggtgaaaatgttgaccttttgttaggaacctgccgttttcctgtgtaagaaacaagaatacatgggcacgcatgtgttaatgggaagacatggaagcctttggttgctaaattgagacgaaagtaaagaggaagtttgaaaagtttggggcaaagaaccctctagaacagaaaatagagatggtaaatttataagtaataggataacgatcataaattagaagtgagttcacccagcttgactctgtgtttctagccatttgggcatcaggctcaggttgggcagagagttggatttatccagatttggtttatccagatctggtttgatttatccagatctggtttatccagatgaagacactggggtagggattggtcatatgattagtggtagactttatacttggataaagaggggagaaaaggggaaaatgagggttgtttttttttttttaaattgttttgtagaaatggagttgcactgtgttactcaagcagtcttgaactcctgccctcaagtgatcctccccaagtgctaggattacaggtgtgagccactgtgcccaaccttggagtgagatgaataggtgatagaatcgctgtattacaggtcctagattaagaatgagaaaacaataaggaattacctaagaaaaataggaaggtacataattggaaagttgagtggtataattgaaaaaacccaggtagaaaacagctggactgggaggtcagtctgaaagcccaggttattagaagctgttacactcgaaccttagtaataactgcagtaagaaaaagatagtaaatggagcagaatccaaaataagatggaaagcctagtttttagaaactgggagagttgatttagaagccacaataaggttagaacagtcaccactgtagggttgcagagaaaagtcagaacaattttgttgaagattcatcatgagttccaaaagatgtggggaaagagttttagaatgtacaaaggtaaatgtgaatttattcccatgatgactcatggatactaaaggcctggggctttctggagtgctcagagtatgggcagaggaaaggccaggaatccatcatttctgagcactgggggaaagaaaatcccaaaatttagtatctttatcttggtgattaaaagactcaagcctgtaatcccagcactctttgggaggccaaggagggaggatagcttgaggtcaggaattcaagaccagccttggaaacatagcaacacatcgctactaaaaatcagacattaatgagacgtggggggcttgcctatagtcccagttactcaggagtctgaggtgggaagatcacttgagcctgggagattgaggccacagtgagctgtcatcattccatggcactttagtctgggcaacaaagcaagactcttccccctactcagcatccccccccccccaaaaaaaaaaacccatctctttacacaaattgcctacagaccacttgatacagaatgggccagacatggtgagttagttgtgaaccaactggttacactggtgggtgtgttgaagagagaaggttttatcattcttaagtttgacaccgttcattgcag

SEQ ID NO: 17 - DNA, 1909 bp, Homo sapiens
gtaaagtgagccatctccaaggatctcttccaccctcttaaactttgctttttttttttttttttggcgacagtctcactcttgcccaagctggagtgcagtggcgcagtctcggctcactgcaacctctgcctcccgggctcaagggattctggtgcctcagtctcccgagtagctggtactacaggtgcgcagccaccacgcctagctaattttttgtgttttagtagagacagggtttcaccatgttgcccagggtggtctcaaactcctgggctcaggcgatttgcctgcctcgccctcccaaagtgttgggattacaggcgtgagcccccacctgcctatctttgcctttttttttttttttctcggagacagtttcgcttgttccccaggctgaagtacaatggcgtgatctcggctgaccacaacctccgcctcctgggttcaagcagttctcctgcctcagcctcccaagtagctgggattagaggcatgcgccaccatgcccggttttgtatttttagtagagacagggtttctccatgttggtcaggctggtcttgaactcccaacctcaggtgatctgcccgcctcagcctcccaaagtgctgggattataagcgtgagccaccgtcccccgctttctttgcctttttaaagtcttgtctgggctgggctcacgcctgtaatcctagctctctggtaggccaaggtgggaggactgcttgaggacaggagagcagcctggccaacatagtgagaccccatctatttaaacaaaattttatttattttttttcttttttgagatggattctgggctagatcgcagatttgggatcgcagattgggggctggatcggggatttggggatgggtggggggcggtgaaaaggtgaaagggagttgccgcagctcagaagccgatggttgggtgtctgagaagattacaggcgtgtgccatcatgcccagctaatttttgtatttttagtagagatggggtttcaccatgttggccaggctggtctcaaactcctgacctcaggtgatccgcccgcctcagcctcccaaactgctgtgataacaggcgtgagtcaccgtgcccggccttatcactactattttgttttgttttgagacagtctcattctgtcgcccaggctggagtgcagtagtgcgatctcggctcactgcaagctctgcttcccgggttcacgccattctcctgcctcagcctcccgagtcgctgggactacaggcgcctgccatcaggtctggctaattttttgtatttttagtagagacagggtttcaccatgttagccaagatggtcttgatctcctgacctcatgatccatccaccttggcttcctaaagtgctgggattacaggcgtgagccaccacaccaggccctattattattattatattttttttggagatgaagtttcacttgttcgttgcccaggctggagtgcaatggcacaatcttggctcattgcacccttcgcttcctgggtttaagcagtgttcctgcctgagcctccccagtagctgggattacaggcacccgccgccacacctggttaatttttgtatttttggtagagacagggtttcaccattttggccaggctggtctcaaactcctgacctcaggtgatccacttacctcgccctccctgggattagaggcgtgagcttctgcgtccagccaccatctctaaatttttaaaaaattttcttgtctggaatctaaagggattctattgagcttaaaggtttgggttcttggtttttgaaaggtgctgccagccatcatcctactgaggggtagggctgggaggtctagcatttcatgggataggactgtttgccctaatttcctttccatcttctgtag

SEQ ID NO: 18 - DNA, 2062 bp, Homo sapiens
gtgagcctctggggactgggttcggagaacgacggcgccgcgcgggtcgagggcttctcggggtgccaccgcgaggcctgcagctccgtggcctgccctggaagcggcctaggcttcctggcgttcctgcgggccgcggatggcggtggattgagtgagaggccccagccaggagggcgctgtgtggccgggttccaagtgaagcgcagagagatgggcaccaggcgccccttctgtcccggagaaggcgtttgtgagcatttgtcggtcaacggaattagtaaacgtggtattttgagggcgtgtgtcatttccctcatgtcttatcccgtggttttgggaagagaaggcagcagtgtattcttgccttaactggtcgtttctgggtacagtccgctggccccaggacctcaacttgaagggttgctgacggttgcttgctccgtctggggaccccgggaggaagctgcctcggatttaacgccttgtcattacatgggatagaactgtgtaaaaatcattagctttaaaaatagacagtttgtcatttcgtatatacacttcgatgtcaaaattaaatccacaacttctcgagagttaggatttgccagctctgttggtaccgtaaagccaaataacatggctgggattctttagacggtgcaggttgtgtctacaagtgagagagcttatatgttaagtctactatctattccttaacagtgacaagtacagtgtctttgctgtgctgaatgaagtgtaaataggccattagagcagtttcttgttttgcatgtggaactgttttaaacccttcgatgaagagatgatgacgagtctgacttggggatgttctctttgcccaggtggcctactctgtgctgcgttctgtggcacagtttaaagagccctggttgaagtaatttcctaaagatgacttagaggcatttgtctgagaagggttgctgcactcctgttggaacaagggtttggaaccattggttggaggtatttagttttggtgagggatgcattgactaataagactagactggctcaggcgaaagtgagcctacaccgatctcctaattttgaactttgctttgtttggattagtttgtcgctgatggcatcttcaaagctgaactgaatgagtttcttactcgggagctggctgaagatggctactctggagttgaggtgcgagttacaccaaccaggacagaaatcattatcttagccaccaggtaaaactcatttgactggccatcacctataattgttataaatgctaagttggtttatttatttatttatttttttgagacggagtcttgctctgtccccaaagctggagtgcagtggcgcgatctcggctcactgcaagctccgcctcccgggttcacgccattctcctgcgtcagcctcccgagtagctgggactataggcgcccgccaccataccttgctaattttttgtgtttttagtagagttggggtttcaccgtgttagccaggacggtctggatctcctgacctcgtgatccgcctgcctcagcctcccaaagtgctgggattacaggcgtgagccaccgcgcccggcaggtttatcctttatgaacttaaggggacagacggaacagtgaatctgggcttagtgaatgtaattgcttcttaaaagctagattttaaggccaggcgtaggatcacttaagctcaggaattcaagatcagccggggcaacatggggaaacccccgctctacaaaaaataaaaaagggtgtggtggtgcatgcatgtggtcccacctattccagaggctgaagcaggcgatggcttgagctcaagaggttcaggctgcagtgagccattggtgtgatccagcctgggtgacagagtgagaccctgtctcaaaaaaaaaagctgcgttttaaaaattttagtttttgtcaatgaaaattatatgctttgtggcagagattttgctaccacacatatatgcaagatttaaagttacatctagcatttgtgagaatcttgaattgtcacttttcccccccag

SEQ ID NO: 19 - DNA, 3256 bp, Homo sapiens
gtaagtgtcaaatctgtgcaggggggcaccaagtaagaggaacagactttatactaacctttaggaagtataggtgggctttgggggctgggcagccagttttcacttaatttttcctgatttacatattagaaaatcctgggaagggctctttagggtccctcaccccccacccccttggatctttggtgcaacaaatactactgatgaaagcaccagtgtttgtgacaaacatctccctatcccaagaaaatcgtgcctggttgcaggggacgcttgacagcacttggccccgtgacagcccctctccgggccaggtccctgccacagtcttcggacatcactgcagtgtcttcaacgtggctttggccgccccctcgtccggcccacgctgatcactgcaaattcaccccaccccacctcacgcagatttcaatgtaatgaaccaacacaggatgttttaaactattagggaaacattgtttccataatattctcaccagaaacagatgtggaagtggcagcccgtgtttacagtttctcattttttttgtccccctttaaattaccattatgggtaaatcattgtttccctttaaaacaaaaaacaaatcagtacccctgcctacagaaaagaagagggaacatttatttaaaggatggaaagactgggagttaggcactcccagcctcactgaggggcatttttgcctttgagaagtcctcccaggtatcctggtatagcacaatgtggcaatccgtagtgggaatgaagattaaacaggagagactttgtattagacctgtgagacgagttaggaagccctgcctggcatggggaatgcaaacagggaacattcaataaatcggggaacttgcttttctccccaagaccccagatggggcctacccatcgttctcgtgtaacaaaacgtgtaaaaaaaaaaaaagaaaaagaaaaaacaaaacaaaaaaactaagttgaccagtctgttttcactggtatgagagcattaaccgcactagatttcaagcacctgaaggcactagtttgtaattactagctggttggcacagtcctgttccctttcttaaacttggattcgattgaaaatttaaccttgagttgcttcagcttccatttcaaaaggggagttggagtcaaggtggtagcgtttaattataccaaccaccccccaaaaaaacaaacaaaacaaacaaaaaaacctttactagtcactaagcatccaggaaacaaaggtgaactttttcacctttcagagcaaggcaggcagctccttatagagaattctagaatttgttagatcaaataattgtttagagaaggatgacatcacccatacgaatagtcctgttagttgaggatgctttttctactttaaccctgaattcagaaggattggttgggatgcacagggggcttcttcactcatctgattatgactctcaatgggttatcaacatgcacatacgtgctattgagtaagtcagcatcacttgtcaccaaccaattccgaaattaattcttgacacacccctcgataaagccctgccagcggagactgccatcctgacaataaataaataaccctgagacagacttccaaccatgaatctgtcatttgaagaaaaatcttgcataaattttccccctgcaacttgcaggagtacttacttattcatgagagtagttaagcaaaaataatttgaccatgtatcctctagataaatattttgatgaaaactggacatggctagcatttttcaaagatgcagcagccactgtttaccttagcttgagtcggatggaagctaagtcacctttgaaataaattactgacagcttcattacagattgccctaaaatttaaaaatttttatagaaaggattctcctccctaagcgagccagcctgtaacaccaggcaagggttttaaaccagaacttctcttttctgttttctttttggaaagaggaagcaacattcccagtttgccttctttgcagacaagggcacagatacgttaaatttagcctgtgagagggtttacaggggtattcctcatgggagcctagataccttatttttatctcatccaactgcctccttttttctttttcctcactgcggtattgcaacaaaagctatttccttcttaactaggaattccctatgagacagtgacatctgtccctaaagggtggtctgggtggtacagggttcttcctccctgatcttgcgttttgttagggctcggctgagctattaaatttgccttaactctctgcgggctgccatgctgattacatttttaagagtaagataatactgatttgaccttcagaaatacagaggttggatgcggattttctaactcaaaactaactctgtttcttacatttagtgccaggaacaaaacaaaaacaaaacataaacaccattaacattggttgaactaagctaatcaattaccaaactgtagaatgctgcccttcggccttcaaggaggaattcctactgtttatgaagatcgggtttgggtttttggttttttttttctttttcttttttccgtggtggtggtgggtgggcttttgttctttttgttttttctgtggtggtggtgggtgggcttttgttctttgaaattttttttgctacatcacagtccagacatagtttaatgacaatttatggggaacaattatttataaataaaaccattttataggtagtttccttccactgcaagaagtgaggtcattagttttgcaggaaatcctgggcagaaatctcccccgtttatgcctttatgccttaagttacaaccttgggaagattctgacctgggagaattaggagccttatgcgctgccatattctccaaagaattgtgagataaatcactgctttagctccctaatgcgtgtacgtttagtgaaagcacaagaaaacaattatccatccctcctgtctccgaccagagtctccagagagctctgttaatactgcagcaaaggtgtgggattcttcccccatggcctgcagtctcagtggatgttccaatttagccagaaaggcgacctaagaattgccgggaggatggatggctggcgcgcgaattgttgcttttgctcttggctgatggttgaggaatgtagagagactgtgtggggtttgtgtgacactgcggtgccttgcagattaggtgagctttcaatctctctttaaaag

SEQ ID NO: 20 - DNA, 3681 bp, Homo sapiens
gtaagtaaccgccacccaacccatcagcacataaaacagacaaaaacaagaaaacatgaaaacctgtgatcctgctttgaaagttacttgttgatgattcctttcttagaagccaaccagttaccccactggcagaaagttctaatctgattcaattgtttatttataaagtccttaattgcattttttttcttttagtgcagcgacattattttacataaacatttaaagcctgttagagcaaaatgatgatgagtttcatagtattcctaattagtaggaagtaattaattgaattgaccctaagcatttagggaaatagggctgtgctcacattttgaataatgtaaaaatactatattttattgctgtaattttaaaacataacaaagaggaatggtgaagacatagctacgcctcctctctccttggtagttgttgattgatctatttctgctctcaggtaagcgactctagtagctgcctagtgttactagatcctagtgactctaggtagagtttgtttttgtgttgtcttgacttttgaactctatttcatatccttatttattccttctcaccgggtcttgattcaaggatggtaagtggtcaatggattttaaatcctaaactgtacacctaagcagtacaataccgtggtggggttattttcaaataaaacaaattactacctgcgaggaggacaggttaaggcctgcagtagcaatgggaaaagcatctttctccaaagtcactcataaaggtgggctcacctggttggtgtgttggcaaatgaagaagggggaacttaaaacacagactttattgcacaactctacctgatgcccaggctgcttcccttgctgcctaacacctgacttaccattgcctcagggatcgccctctgtccctgtcacccaccatggtctctcccagaggacatcgtacccgatagctagaaagttgcctttgaaatagtttatggcttccctcatcttgcacatgatatagcaagcatgtggtggtcagggttatttaaaaaatatgtaaacattattattatttttttgaaacggagtctcactctgtcacccaggctggagtgcagtggtgcgaccttggctcactgcaacctctgcctcctgggttcaagtgattctcctgcctcagcctcccgagtagctgggactacaggtgcctgccgccacaaccagctaatttttgtatttttagtagagatggggtttcaccatattggccaggctggtctcgaactcctgaccttgtgatccacctgcctcggcctcccaaagtgctgggattacaggcatgagccaccgtgcccagccaaaaatgtaaagatatcttctgtttgttgatgctgcagaaatgcacaaagcagtctcagttaatgatattctgaagtaggagtgtcctgtgacttggggcaggggagtctagtagaggtttgatgttttagccatccttattacaagtagctgaaactcggaactgattttgctataggaagtattagctaatcagaaccaaggtgccaggctgactcaccaagggctaagattgctttacttagtagcctcaagcccaaggaactgattgtgaaaaccacctgaataaacaggagggaggaagaggtaatactgttcatctataaatcatataagcctctgttagggtctgcgcaatctatcaaccccagccctgccttcccataggaaattcctttattttcaattgccacatacatagatattccacggtttaatatacaagcaaatgtgtatattttttcaaggaacagaaaaaaacagtccatcttggctggtccctatggcccccagcccccactccttcttcaacaaagtccctgattttctcaaaagttcgaaccaaaagctggaagcgctaattatagtaacgcaaaacaaatggaaatcctggaattgtttgcattttgtattttggatccaagtcagaagttaagtacaggtacaagattaagagagtttaaccaccccagttttgaatgctgggggggcagggagacccagtttcgttaattaacagtagcttagccagattgttgaattttgtcgggtttcgttttctctctcaaatcatttagaagtttttggggtttttttaagcaacacttaattactcctgaaactttgtctgaaaacgcaccatttgtatagatcatgaaaagttttaaggaaactcagagaaaaagagaacaacgcagcttaaaacttttaaaatgtcctccctcacccgtggctcaaacagccctgcatctgccgtggccggcacgtttctggttgaactgcctttatgttaaagttcagatactggtagtgtgcccatttcttaagctgtctatttttatttgttgagctggggtttggctggctccactccagatgtctctctcacaagatttggtgctgatgatctatttatagaactgtggttctgttgccatggtaacatgctggaggccagggcggctggggagctatttctggactggtgctgtaatgtaagattgattgggcaagttagtatatcctctaagccagactaactctgaactagtaaaaaggaagaggggaacagaaaacttaggcagtttctttaaataaacttttctctctttatgattttcttttctcgttagcccgctttaaaacaattccaatctctacatgcccctccctccaaaaaataactggttttaacattaattttccatattaattaccccaatctttcaaaagtaaattttcctgtgtgtctagtcaagcaacacaaacaagatgctttttttaatgaaaagcgtaatatctggagtgttctatttcatggaccaaacagaacggaagagaactttgtgtttgtttcctgttcaggatggctagggcgagaagggcccctttgtgccaacctcttttgttctctttaatcaatgagattcttaaaaagtaaaaaggaagggatacagattctcagtaactaaaacaatctcgtgtgcccttgaggggaaagtccttgatgtttttaagaatgtcactttattgttttttaactgaatgatatagaggtatacaattttcaaactgtttgccattttaatcaagcaatttgaaaattaaaatgtttttgtcaggcattaccaaatggcacagaatgtgataggccagcctggttttggggtcctttctctactggttgatatgcataaaaccttctaaaaatcaagaatattgccagagagcaacaggaataaagaagctaagtaaagaataaaaaagaaaaatagaaaaaataaaaataaacacgaagaacaaagccccaccaccgtgctgtgctgtttgtgtggccccactgcgtcgaggccacaggctagctgctagacgcatctagagttccctgattcctaaaattatttatcttaaatcctgtttgccctaaccttcttaaggcatcagctttgagttacaaatgtaaccaacacacaagcaaatgtgccattgacttagtgctgcataactgtgggacggtcacttccgttgagcctgaccttgtagagagacacaaatagttggcaaattgatgtgagcgctgtgaacaccccacgtgtctttctttttctcccag

SEQ ID NO: 21 - DNA, 2782 bp, Homo sapiens
gtgagggacgccccgaagcgccgggcgggcgggggcggggactccgtgcgtccttccatctccccgggcggacccctccaaaccacccccttccgggccgcagcgcaactcccgaggcctccggctgctctgctccacgcgggcctgcagaacaaaggcgcgcgggctggctggctggccggccgggcgtgcgaaccgctacgcgcccccgcccgctgcgccccggctggccggcgcccaggctgaactcacatcctccgcaggccgcgcctccgcggctgtgctcgcggtgggagtgggcctggggggtcgggcactgggcaggctcctggccctgcgccccgtccccgaggaggaggaggcccgggctctccccccacagctgtttagagagggccctctggccttcagaggcccgttggttctgaagtgtgggttggagctcgtgcgtcccattgtctggggctctgggctctttgtgtccctggaggatgggtcgcccatcggagatggctccaaccaccagtttagatctacccccccacacacacacaggcaacctgggggccagaaagaagcatagttagcaaaactgcacccggggtgagttgggcgtggttgactgagagacctggcttgtagactacttggacgccaacttttctcttcatcttgggccaaactcttgcttccatgtaggccttgcttttctgcgtgtagcacgcaggagttggttggtagatgctccagcgtgcattgtgccagcccagtgtctgtgtcactggactggagaggctggcattgaccaggcctgctgcagcagcccaattcgttctcaagataggggctgcatgagagggcaggccccagaatgggtgactcgggggtgatgtggaacagggagaggagagggatgatgagtcttctcttcttcagaattgagaggccttccagttaccctgcagccctggaatggcccttgctgaacccaagaaagtgagggaccaagacttgggttcccccagccactcagagggcagatcccaagaaccaaagttctttctttggaatgccagttgaggcgaggggcaggcccacccccacaagtcccagcctcagtctagccaagctgagtcatctcagtcctggatggtagggggggcgaccggatgtggggaggcgggccacaccccagctctttagggggggtctcctccctcctcccagctcccctcctccttgtgagctggctggagcaggcctagttcccagagctttgccatataaggcaaaaaaatgttggaaagcagcagtgattaggggagggaggggttggctggcccaggggctggggaaaggggggtgggatggggcggggccatccagccagacagtctcccagccctgggccctggtccttccctctctcttttgggagggggatggggacggagtaggctggcctaacatggacctggtgggcctggttgctccccgtgtgggggtcgttgaggggtctgaggctggactggggtgggggcctgatgggaagccctggctggccttgttccctggccctgcacccttggtcctctccgagtccccctccctgctgccatttggggagagtgaaaggcagcctcgtggggccaggaccaacctgggaactggggaggccactctgagcggctcacctggccctccaggtgctgcctgctgggctgggaagcagctgccatggacagcccagcccctcctcataggcccgccccctagttgactcaccagagaagtattttgtctgattatgggggaggctgacagggcttctaaagatgagccatcctgccttgagcctgagacccagaagtagccttgggtggggcccagagcccagcagggttgccatggtgatggggggtgtcagggacccgccctaaccacttcccctgtggcgagcctgccgaggcccagaggcccgttaggtggcaagtgggtcaggcagtgggtggcaagggggttcccagtgtctcctgctgccaccccggcatctcccgatgggccccagtgagtcagggccatgtggcccaactgctggttcctttgggctctgggcccctcccctggctaggcctgtggcctcccaactccaagcttgagtcttcgaggtggtagaggaagtgggggtggtggcgggggtcacaatccctcacccaaagggcgtttgcccaactcccccacttcccctgcctcaccccacccaccctggaacaacagttgtatttcccaaccacccagggccacccaccttcctcttcctcagccccagccccactccaggtcatgggccctggattctgggccagctggagtcagcctcctcctcctggttccccataggcccctgaggtgtgtggcggacccctgctctggcagctgtggacactgtccagctgttctccagagctgcccatctgcaggctgggtctgccctctgcccttcctctgggcccagggcttctggcagtaatcataccctgctggcatcgtgtgtgtgtgtctgtgtgtatctgccctcaccccaaagtctccttcacctctgaacaccccctgggaccctgggcagagagggcctcatccacatcctaccactggccaagagggggtgtctacctcctccttggcccctgctaccccgtgctgccagaccctgaccccaaagcctgggatcggcccctcccgcacagccgtgtgcccagcgtggccactcttctactcacag

SEQ ID NO: 22 - DNA, 3144 bp, Homo sapiens
gtgaggtctccgcggcccggcttcgcgccgtagggtcgccgcgctcctcgtcggccgggggcggggttggagaaggcgggcagagaggccggaaaacgcaggcgccagctcgcgcccaggtccgggccaggttcagctgggatgcgtgagccgatggaggctcccctggcatctgggagtgagtgtccagagaatagcttccctagttgacccacgagccatgtctgtgcccacgaatgtgggagcctgaggccatggggggtccccgagagagagagggtgtctgggcacttgcccatgaacggaccactgcatgcctgagagagaagggccctgacaccaggaacttgtgtggactcagggcgagtggccacctggaaggacacttctcagccagtgtgcccactgggagggggacagcgtgtcccttgctggctcgagtccacagggcaccagctgctttgggcacaggggagctggttctggggctgctctactccatgggggccatggtgaggatgagaccaggccaggttccttggttgggccctgggccctatgccaggttggctctgagttggatgcgcactgccagccaggatataaccaacaatgtccccagattcccgtgccacctggggccaggcaaaataggttaaaatccactggcagacccatttgtaagtgctccgagtgcaccgtcaaggtgggagcttgggaatgggcgacctcacctgccgcacctggacttgccagcacaggtctcttctcaccttgccagtctgcggcttccttccctcctgcgccccctggctacgcaggcctggaaacagctccttagggatcccagctgaacagctcctgggcttgggctatacctcccagtgggagggtctggcctggggtctgagggccaggggtggtgtggtgggcggggtgtgcctaacatccaagaatctactccaaataagggaaaatatgaaagagctggattttggcttcccaggactgcccgatctggtggctttgggatcgggcgacatctggcggttgatactatgttctagggacaagaaccctctacacgcccattccttgtttcctctaaatagggaaaagctagggctggaagacacggcacccacctcctgacactctttctgctggaattgaccactggtcactctgactcagtttccctgagctctgaagattaaggaatgaacccagacaacccagctcaatggcccttagtggagagagagtcaattattgatggaattccgtgcctgggaacttgctttccagtggggcagggggaccagaaggacaacttcaagcccgtcttgaatggggcccgtgcagggaggggtctgtatgggttcccccttcccagccctccctcccacctccaccccagctcccagacgcccaggcgcacttggtgcaggtgtgtctcaaagtggtcaggagaggggacaatatacaggggttggcctcagtactggaaaatgccatttgcaccccctaacaccactactagtgggaaaaaccagtggggccacaggctgccccgaagtgaatgtgctgggcggatcacagccccaccacgtgtcatgtagacacccagggctacaggagagtcaaccatatttgggcatgacgtgctggccaacccagggcctccatctctcactggtctttagcagataaattaattattactagaattgaacaggagggacagatacctgccttcctagaaaaaaaaaaaaaacactcagaggaggaaacattctggagaggggaggtaccgaggactgagagggaggggagtgcacaatggggaggaagccataggcaggctggcttctgactgaagatagtagcagtgagcgtccccagccgtcccctggtcccagtctacatctcggcacagcagccactgcaccatctccagagttatctaccagaaaattttgagtgaagagcccccacagctctggggacacaaccccccaggcccctgatccctgtcctcaggcctcctctgcacctgccaccacacctgaagcccacccagacccaggtgctcagagcctgacaagtcttatgggaacctctgagcaggcccctctgggtctggaatgccactcccgtgtttgccccctgccctggccacgctgggctgaggggactttccgacaggagaggaccccccgcttcagctccccccagtgccccgggtgaactggcatgaagggttaggcgggcacacgtctctgcatgcatcgcacggcgccctctgcccgcgcccactggcgtgcccgtgggcatgcccggaccccttcgaaggctgcttgctcctggagtgccggcggcctcctggcccagtgccccatatcctcccgggtggagggaagcagagagggaggcggtgggaagcagcgggcacaggtgcgcgggcgagggccctacggactgggaaggcgggcccgtggcagggggtctgcggggaccgcgtaggcgcaggccttgccctgcagccgcttcccaggtgatgagcacacaaagcgccctcggcagtgcctggtccggggctgccgctctccaggggttcgctcttagggttggccccttcccgccgctgccgggccggcggacggggatgccgccggggaaccctaggccctgggggcccggggaagggggatgacggcggggggtagggatgtgcagggacggggcagagcccaagtgacttagggaagagagccaccccaccccccgcaggggccctggggaaggaaagtcccttgtaaggcgcgccggctgcaggccaagcagtcccgcgccagacactgctcagcctttcctcgctctagggcgcgtttcctgcagcgccaacgcgaggctgcggggctgggagcatccttgcccaccccactgcccagcgacccaccctggagcccgggtttcgcgtctgatcagccgaacccgggggcctagtgggggcattccaacgggcggggcggggtggggcaggcgcgccctcgaagccccaccccacggcccctccccgcaggaggcccgccctctggccccgccctcctcccgcatttaaagggctcgctctctccgcag

SEQ ID NO: 23 - DNA, 2334 bp, Homo sapiens
gtaagtatccctttcattagctgtgtatgaattcaggtaaacttttttgagatggagttttgctcttgtcgcccaggctggagtgcaatggcacgatctcggctcactgcaacctctgtctcccagattcaagcgattctcctgccttagcctcctgagtagctgggattacaggtgctcactaccacacccagctaattttttgtatttttagtagagacagggcttcactatgttggccaggatggtctcgaactcctgacctcaggcgatctgcctgcctcggcctgggattacagatgtgggccactgtgcctggccagataaactactttgaagtggaagaaagcttttttttttttttgagacagggtctcactgtattgcccaggctagagtgcagtggcacaatcttggctcactgcagccttgacctcctgggctcaggtgatcctcccacttcagccacctggctaattttttttgtagagatggggttttgccatgttgcccaggctggtgtcaaactcctgagttcaagcattctatgtcagctgcccaaaatgctgcggttacaggcatgagccattgccctcagcctgtatcttaaccttcctttaaatagtctgtcaagttacacagtgagcacaattgcttgtctagaacagtgggtagttctcagtgtggcccccagatgagtagcattaggaactgttacgaatgcaaactgtcatgtctaccccagacctttgagtcagaaatgggagtgttggtctaaaaactgggctttttgggctgggcgcagtggtgcacgcctgtaatcccagcacttgggaggccaaggcaggcggatcacctgaggtcaggagttcgagaccagcctggccaacatggtgaaaccccgtctctactaaaaataccaaaattagccgggtgtggtggcgagtacctgtaatcccagctactcaggaggctgaggtaggagaatcacttgaacccgggaggcagaggttgcagtgagccaagatggcgccattgccctccagcctgggcgacagagcgagactccatctcaaaaaaacaaacaaaaaaactgggctttttttttggcaacccttggggaataaaaacctattattttcttaaggacaaagtatcctgaagcaaaaaaacccaaacaaagaaacaaaaaactttaacaagcactacaggtaattctgatggactaaagttttaggaccataagtctggactatattgaggtgagaagaaactaaactatgccatatagaatggtacttagagagtaattcacatcctgttacgttgtggcatcactgatagaaatattggataatgaaacttctagaagagtttgaatgttcactggcagcagaaaattgacaaaagggtttgaatgttatttaaagtgcagctgtacattcaacaggaatggggtaaaaaagaaagtgcagatgtagctgatgagctgagaatagtgaaatgtctacatgggggaaaaaaggaaagagttacaattaaacctctctaggttagttatttccctgttgtatgtttgccgcagaatgtgctgagtatagcaagcatactatgtatagctctaacctgggtgaaccagaaagttaaacatgaaattgctaatgggggaggctgcacatgtgtgggggcagtggatttatgggacctctaggtacctttctcttaattttgctgctgaacctaaaactggtctgatttttttttttttttttttttttttttttgagatggagtctcgctttgtcacccaggctggagtgcagtggcgccatcccagctcactgcaagctccgcctcccgggttcatgccattctcctgcctcagcctcccgagtagctgggactacaggtgcccgccaccacacccggctaattttttgtatttttagtagagacggggtttcactgtgccagccaagatggtctcgatctcctgaccttgtgatccacccgtctgggcctcccaaagtcctgggattacaggcgtgagccaccgtgcccggcctaactgctctgatatttttaaaaaaggtgacttggattaaagtatgcaaactcaaggagtagtacgagcccacttgagtgaagttagcttagttgctaaagagcttgataccaaaattacttttgtttacttgatgaatgggatagttgttgcacagggccactgatctagattactgtcttatttgtcaagtacttatagttgtagaagtagcagtgtgaaaattatcggaatggagaaggggcaatgataaaacaatttattttcag

SEQ ID NO: 24 - DNA, 2135 bp, Homo sapiens
gtaagtcttttttcttttaattaaggttggggattgagttaaaaataacacattgggtaaatcagctcttctatgtcaatgttaggttaactattgcatttcacataatttgtggttatggatgttcatttttatagcatttaaaagcagtgctaagatagtaatgtctacagtggtttgatttccctttataaaagttttgggaataggtacagctcttcattttttatttatttatttatttatttattttcgagacagagtctcgctctgttgccccaggctcgagtgcagtggctcagtttcggctcactgcaacctccacctcccaggttcaagcgatttctccctgcctcagcctctggagtagctgggattacaggtgcccgccaccacgcccagctaacttacgcattattagtagagatggggtttgaccacgttggccagggttgtcttgagctcctgatctcaggtcatctgcccacctcagcctcctaaagtgttgagagtacaggcgtgagccatggcacccagccaatttcatattttgtcaggccacagttttaaaggtctcgttccatttaatcatgtatgagaatcagagtcgtggtattttatgtcttgtcctgtggaacagtatgttatattccaagagcacttagagtttatttttttagacagagtctcactctttcttctagtctggagtgcagtggcatggtcttggcttactgcagctttcacctcccgggttcaggtgatcctcctgcctcagcctcccaagtagctgggactacaggcacacgccaccacgcccagctaattttttaaattttttgtagagacgggattttgccctgttggccaggctgggagcacttagaattttatcatgattatgtttcaggtatatttataagtgtgggcggcaaaccacccaggtgccaaggcaagagaccgaggacacgagctgttccaatataataaaatataaaataagaatagttataccagatatagatcttagatatgattatatatgaatatcattaatcattagtttgtagcaattactctttattccaatatcataataatcctcactctataatcataacctaggaaaaaccaggccatacagagataggagctgaggggacatagtgagaagtgaccagaagacaagagtgcgagccttctgttatgcccagacagggccaccaaagggctccttggtctagtggtaacgccagcgtctgggaagacgcccgttgccaggcggaccgaggtctagcagtagcgtaagtgtcaaaggaaaacacccactatttagcagaccgggaaagggagtctgcttttcccctggggagtttagagaagactgctcctccacctcttgtggagggcctgacatcagtcaggcttgcctgcaattatccagaggcctaaccgtctccctatggtgctgtgcttcagtggtcacgctcctagtctgccttcatgttccatcctgtacacctggctctgccttctagatagcagtagtaaattagtgaaagtactaaaagtctctaatatgcagaaacaatggcgtaagctgtctttctctctgtctcctctccctctctgcctcggctgccaggcggggaagggccccctctccagtggacacgtaacccacgtgaccttacctatcattggagatgactcacactctttaccctgccccttgtgttttgtttccaataaataacagtgcagccagacattcagggccactactggtctccgcgtcttggtggtagtggtcccccgtgcccagctgtcttttttttttaaatctctgtgtctcgtgtctttatttctgcactctctcgtcgctgcatacagggagaagcccaccgaccctgtggggctggtcgctacatataagtactcgtccttgtatgaattaatgtttttacttattgatgctctgagtctgggaaaatggcaagtgttaagactcagctttaaaaagttttgtcactttttagtcttttatgcaaacctcagatcgaagaagacagcataaacacttttcactcagtaagttttcccagttaatgtatattcacatctttaaaatgcag

SEQ ID NO: 25 - DNA, 2171 bp, Homo sapiens
gtaagttataaatatctagctaatcatattttgaatcttatttttaataattccaaatccattcattttaagttaatatttgtcagggagaagttagatgcacataagataaaagaataaaaacattttaaagatgaataacaaaaattcatgtagttgtactggagtgaaaattaataagctaatcttgatttttacaattgaattttctacacagtaagtgtaacatacaaataacaggtttaatataattaacattcatgccaaaaggaaaacagaagataagtaaacagaaaaatgtagctttttcaaaatgagtgcattagtgaaaaatcaggaattcagacaaacctaatattcataattcttgaggtactggttaaattaacccattttataggtgaggaaactcaaagtaatttgcttttatgtactgtaaaatgagagacatacattactcacaagtccgggcgcggtggctcatgcctgtaaatcccagcactttgggaggccgaggtgggcggatcacctgaggttgggagtttgagaccagcctgaccaacatggagaaaccgtctctactaaaaatacaaaattagctgggcatggtggcgcatgcctgtaatcctagctactcgggaggctgaggcaggagaatcacttaaacccgggaggcggaggttgcagtgaaccgagatttcgccattgtactccagtctgggcgacaagattgaaactgtctttaaaaaaaataaataaatactcacaaatgcctgttaccagggctttaggaggaaaaaaaagcaagcccaacactggcaggatccagaaaagaatgatatgttgctgttttgagtaaaagtgatataactcttttggaaatttgtaactgtcctaagaagaaatacacaaagtgtctttcataaagtttcagctttactgcagtgtgcctttattcccagccactgggaagctgaggcaggatgcttgagccaaggctgtagtgtgttgtgattgtgcctgtgaacagctattacactccagccagggcagaatagcaagaccttgtctcttaaaaaagttgcagcattatttacaaagcagaaagtgatggtcacaatgaaatacgctccctttgagaacatttatgaaactaatagcatagcgaagaccaaccatattttgtggagagataaggaaattatttttattttactttttattctaaagctgggttagagataaggaaatttagatgactagggtgagattatgtaagatggtttttccttctgctttgctgtgtattataatatgctatgacacatgtgctacctttttttcttcagtgatcgttgttctttttatatttttgagacatggtcttgctctcttgcccaggctggagtgcagtggtgcaatctactcactgccacctctgcctcccgggttctagcaattgtcgtgcttcagcctcccaagtaggtgggattacaggtgtacaccaccacacccaactaatttttgtactttagtagagacagtttcaccatgttggctaggctgaactcgaattcctggcctccagtgatcatcccaccttggccttgcaaagtgctgggattacaggtgtgagccactgcctagcccagtgatccttgttctgaattactactgttatattcagaaaaaattaagttttagtgtcattttgttgaaaatgctgcagttatgtcaggctgttgtgtgttagagtacacgattgggaagtttaaatttttattctaggagatggcaaagaaagcgcaagcgccactttaaaatgacgatgaaagctttttaatttgatagaggtctttttattggcatggtacacatgcacttccactcatttgtattgatgggagtgatccctttaaccctaggttagctagagaagagaagtgtgtattaaaagaagtggttttttttttgtgattgtagttagaggcttattcatatatttcatggtgtcatatcttaaattatggccctatttctattttgtagtgtgctttttattagggattctgaatattctcattaacagtaacaaaaccaaccatcaggcttacttaatctgaactttataaaatagaatctaatgttatagtccttgtatatcttattag

SEQ ID NO: 26 - DNA, 2280 bp, Homo sapiens
gtaatgtgtcttaattaaatttttacacacaaagctctgaattagtgctcagtgttaattttagctattactttatactaaaaaagtcaataaaattagacttacatggtactttaaaatatttgtggttatttgaattaagctgctggccatctattacactacaaagtagtataatgcaaataaatagaattctatccaggttaaaggttaatagcattagattagcacagtaaggtatgactaagtaattagaacagattttaaaatattttactacattaagaggctggagatatatatgtgtgtgtgtgtgtgtgtgtgtgtgtgtgtgtgtgtaattagagatcccatgtcatcagaagcatatatttgacacagccagcctacagtggaatgcagtctcatagaacaataaagctggtcagtctaagtaacatcatgtttgagcagtttaaaacaatgactttcagcgatgaaaccaaaaattcagattttccagtgattcaaagtacttatttcctgcatttaaaaataacttcataaaacttgaaattgatgaagcacaaaccagccttagtttaggtaggggtataacagataaaataacttttaagtgttcatgtgcagatgatttgcttgaccccaccctttgcattttgtgttgaacttagaggtgtcttgctttttttaatatgtcaaataataaagaattaggaaatctaatacttttttttttgactcttgctctgtcacccgggctggagtgcagtcacgtgatctcggctcattgtagcctccgcctcccgggttcaagcagttctcctatctctaccttgagtagctgggattacaggtgtgcgccaccatgcccagctaatttttgtatttttattagagacggggtttcaccgtgatggccaggctggtctccaactcctgacctcaagtgatccagccacctcggcctcccaaagtgctgggattacaggtgtgagccactgtgcctggccaggaaatctaatatcttactgccatgctagcatgtcacatcaagccctccactagagcaaaaaagtaccattttgcacaaagatagatgatataaaacatagaaagtgacatcatgtttacatctcaacccccaaatttagctactgccttttatatatttcaactgtcatgcttcttaatgtcttataattagaagctgtttgtaaaagttgatgttaactacctaccgtatgagtaatgctgcataagttgaccacaaatatgtgaatatgtcatattttcttgactttggcctcaggagagaggcacattcagaagaaaatctgccttggcccatacatcttctaaatttctgtaatcaaatgtgatttttaaccttttctgccccacttgcctgagataccacccgcctcagttacctgtctctcctactttagagggagttaggggaaaacatgtcttcagaaataaatgcttcctcttccaggtagttcacctgtgtctttaaaaagaattttttttccctgtctttcaaattcctgtattacctatgtcttaccaaagagtccaactatgtattccttccactcctgttgaaagtcgggtaggccaaggcttaataaatacttgtttcttaattgatacaaagaaaatcaaattggtcttagcacaatagctcatgcctgtaaccctagcgcttggggaggtcaaagagggaagatacttgaagccacaagttttagactagcccgggcaacatagcaagaccctatctctacaaaaaataaattagccaggcatggtggtatgtgggccagtagtcccagatacttggtaagttgaggcaggaggatcacttgagcccaggagttgagttaaagtgagcttattcattgtgctgctacacttcagcctgggcaatggaatgatatactgtctctatagaaaagggaaaaaatcaaattattgggtaaaaaatgcaactcttttgtgctagagctgagaataaaactttttacagccgtttaaagtgtacctccttaaatggaaggaggaatccttcaggggagggaaggtgtttaccttcttaataataaaaggtctttcaatggaaaagtatacctacctggggcatagaaggctgggagggaggagtcaagttacggagctttgaaatttttttcatggctttgtatgttgaaatttgaaatgtataacgtgaatgttgtatggaatatctttgatttatgtaaaaaaattttttag

SEQ ID NO: 27 - DNA, 2630 bp, Homo sapiens
gtaagaaaacatatggttcatttatttgaggtctagtaaaaattcatacatgtagatatatgtatataaattttgtattattatagccactagagaggtgtttgcttcgttacttctaaaacttaaattcacatttgaactggttgtcatagcctaatggttaacttaaaaatggcaagcactgaactctttgctaaggaaggctgatatcctcacatgatatgcactattccaacaactagtcattttgctaaagcaagcaatttattagggacttataattgttactatagttctgatctgtttattcatttctacaattcattctgggtagtcaagccccagttccctcccctgccccctagcagtaagaatagagatcatattttcttttatttgaaatcagagattaagaaacaaagcagtagtattctagtacaaattctgaaatcttgatttaagaagttctcttctagatgtttttgtctagcatccgtgggccaagagtccccctatatgcaaggttttgtgcatacataattttgctggggttggggaagagtgcatagaaaaaaattacaactcaactgaactggaatgacgctgtggctgatctttctagtcagcttttcctggcaagtccgaggaagcttctgtgagagtgaggaaatgttaggggaaaggtggttgaatacaagtaagatggaagtgccatggttgtcttgtgtttcagattatgctttttatgtaagagtattggagtacgatagaatttattcctgaatgtttttgtcttcagtgtgttccctggatattgtttaaattgagtgttgtgcatcagcatattttgaatgtatgaagcatgcatgtttttctgtttcttgagacaggatctcattatgttgcccaggttggagtgcaggggcatgccacaatcacagctcactgcagcctcagcctttcctgggcttaagccaagcttgtccaacccgctgcttgcaggccacatgcggcccaagaatgcttttaatgcagtgcaacacaaattcttaaacttatgaggtgttttttttttgtcttttttaaagacgagtctcactctcgcctgggctggagtgcggtggtgcaatctcagctcactgcagcctccgcctcccgggttcaagcgattctcctgcctcagcctcctgggtagctgggattacaggcatgtgccactacgcccagctaatttttggtatttttagtagagatggggtttcaccatgttggccaagctggtcttccaactcctgacctcaggtgacctcctaaagtgttgggattacaggcgtgagccaccgtgcctggctggttgtctgtctttttattgattaagggaacatttgttttatttttttgagatggagtgaagtggtgtgatctcagttcactgcaacttccaccccctgggttcaagcagttttcctgtctcagcttcccgagtagctgggattatatgcacctgccaccatgcccggctaatttttgtatttttagtaaagacggggtttcgccacgttggccaggctggtctcgaactcctgacttcaggtgatccacccgccttggcctcccaaagtgctaggattacaggcgtgagccaccgcgcccggccaaacatttgttttatattggaaatattttcttcgaattttccactttatagacctcactttgtgtgtgtgtcatcttagaaaggttttctctctatttctacattctatgttcagattcagaatctacctggcatttaagatttttcaatgtaatgtgaattaagagttctacattttttcatttgtcccagtgtgtcatgtattgagtagcccaaagtcctttttccccagtactttaaaatgttaactttagcacagtgaattcccataattgcttgggtatatttctggcctctgttttattaccgtttgttcctatgccactcgcatactgttaatgtagttttatagcatgcttatatatattgctggcctagcgagtggttctttgcaagtcttcattgctcatttaaaaggtctttgcaatcaattttagaagaaccttcctcttgagtgttctttataccgtaatatttatggatcctctgctcttgaatgtaaaaacttttattttaagatgtgggtattgtaagattcaactacttttttctcaaaggcatagtgaagcatcttgtcagcttgaattgttagaatagtcaaaatttaattgggaaatgcttccagtggtgcccaaaagtcaatttagtaggcctgggatgtatttatttagataattaaatttcaatattgaaaatccaatcaaattcagtatctaaaatctttaaaagtttctgtgcagccttcacttctgtcagttctctgacctggctagggtaatgcatatgtgtagtttgcccttaataattgggtattaaggaaaaattgtgaactcaatgggtaaatacaagatgattaagcattaacagggtttagttatttgattcagtgatttataattgtctaaattgtaacgctctttttttgctcctcattctaccattacgttaactttttttttcttttttttaatag

SEQ ID NO: 28 - DNA, 2251 bp, Homo sapiens
gtgagtagggtaggaggttggggctttgtcgtctcactttcctcctttttctttttttttttttttttgagacggagtttcgctcttgttgcccaggctgtagtgcagtggcgtgatctctggcccaccacaacctctgcctcctgggttcaagcgattctcctgcctcagcctcctgagtagctgggactacaggcatgcgccaccacgcccggctgattttgcattttaagtagagacagggtttctccatgttggtcaggctggtctcgaattcccgacctcaggtgatctgcctgcttcagcctcccaaagtgttggtattacaggcgtgagccaccacacctcacccctcctttttcttgttcccattctttctgtcagagatctttctgcctctgccagaagcccttctttttggcctttctttccctcagcttctgttttttttttttttttttttttttttttgccttaacctctttttagtgctggttgggacagtgtgtctggggtcctcaaaactactctcaagctcaattcacctagagtacttacaggactcagcatatagtcgtgcttgtggctacaatttattacagcaaaaggatacagaccaaaatcagcaaagggaaaaggctggtgcaagtttctaggagtcttctcccagcgggagttaaacaaggcggcctttatttccccagtagcaacagtaactacagaaataacagcaacaaggtgtaacaagacatgaaatgttgccaaccagggaagcttgttagactcagcaccacctcttttacttggggctggtcatgtaggcgcccccttcctggcacataacaaaatttccagactcccagaaggaaaggtgttcagcgtaaactgtattgtttgcatgaacggtttaggcacagtgaaccactgataccaggtagggagcggtgggaaccttcctgaattccaagttcccagacaccagccaaggagccgaccttggaagcaggcctttccaagggttgtagtttcagtctcctaggcttacacttctctgcacatatggaaactgggttggttatgcagggttctctgagaagctttctgtaaggagcagtgtcgtttttgcttacccaggatttattccccctttcatttatttcatttattcaccagatagttttagtactgttttaggtattggggatataccaaggaataaaacaagattcctggagggagaggggaacggacagcaaacaatagacacaatacgatcgtgaggtatatagttaatgttggtgatgagtgctcctcaaaggaaacaactaaaaccagacaagggaagtcggggtgtcagggagaatggtttgtagtattgatgacaatggttaaggaaggcctgagttctgcaaagacctcaaggatatgagggactagagtgccctctgctgtggaagagcctgcgcaaagatcctgaggtgggaggggtcagggagccaactcattttacggtaagtggaggtgggatccaggggaggattctgagcagaggagggacgtggcccaatttgggctttgaaaggatcctgcttgtctgtgtgccagcatcctggtggtggttgggggagtctgcatcaatcctgggtgctgttacctgaaaaaatagcaggtgttagacgaaagaacagatgtctggcccattgtctccagtctgttcctgtccacagcttccctttatgctgagcccctgccacatgcatcatcatcctggccatgtggcattgtcacagaccgtctctcctgctagcctcctaactcagggcctgggtcttccccagcattaagtaaatcagtctcaggcagacagccctttataggtgtttgggccaggtgcggtggctcacgcctgtaatcccagctctttgtggggccgaggtctttgagctcaggagttcaagaccagcctgggcagcatgacaaaaccctgtctctaccaaaaatacaaaaattagccacgcatggtggcacacacctgtggtcccagctactcgggaggctgaggtgggaggatcgctggagcctgggaagttgaggctgcagtgagccgtgatcatgccactgcattccagcctgtgtgatggagagagaccctgtctcaaaaaaaataaagatgtttgttgggtgaatgcacaaataagtgagctttgttcccccatccccaccctggaactgcactttcttaaccctttag

SEQ ID NO: 29 - DNA, 3178 bp, Homo sapiens
gtgataagctgtgggagaagggaaagaggccaggcatctggccaggctgctgcctctccgcccccaggcccttgccttgccttcagatgttgcatgcctaagtgtaagatgtgccttttctccaaacctccctccctcacttcccaccaggcagcctaggtttctagaatgactcattcttttttcacttttattttgatttttttttcaacacatagacaaatattaagaatattatgatgaatactcaaataccattcctcacctaaatttaacaattcttaatgttttgccacattttcttcttagatttatgtagatttttcttctttgtgcttaaatgtgtcacagacatcacagccctttactccctactatttcagtgcctctgccaagtgaggatatgttgctgtagaacagggtttcttagcttcagcactactgacatttggggctggttatttatttgtcatgggagaggggatgccctgtgcactgtaggatgtttctcagcattcctgacttctgcccactagatgccaatagcactctccctccatcatgacatctcaaaatgtctccagacattgccagatgtccccaggggtgccacatcactcccagttgagaactacttctgtctaacagaaacatcattttcatgctcagcaaaattaacaatcattcttaccatctcttctagttatttatttgtctctaatggtccatattcaggttttccccagctgttactcaaaatgagtttaacagctggtttgtcaggatgattctgtgtgtatggtgcatttatctacagaacagtgcatatattatctagctcggggtgtccaatcatttggcttccctgggccacattggaagaagaattgtctcgggccacacataaaatatactaatactaacgatagctgatgagctgaaaaaataaaagaaaaaaagatccgtgcataaatctcataatgtcttaagaaaatttacaaatttgtgttgggcagcattcaaagtcatcctgggctgcatatgggccacaggttagacaagcttgatctagctgaagttttacaaagtgaacacacaggtgaccaccacctgggccaagaaacagaacctagggattaccattgtgctgtcagaataattcttagtgcagtgtttgaggccaaaccatttagaatagtaggagtatgagggtgggaacaaaatagggaagggaaaaaaaaaacagttccatctgtcactttctcccttctgacactgcagttccagagtgggtatgtttgtatttgtgcaccaaccacagtcttgctaggtcatgtgtaaccttgaacttaacctctctgtgccgcagattcctgttctgtaaaatgggaatgacagcatctacctcacaggactattgtgaaccacttggcatttactgtgtgcctggcactgttttaaccaagctttaaatatatatgtatttgctcatttcttcctagcaaccccatgaaggtaggtatcactgttgtaacacctgttttagagacaaggcacagaggtaaagatgttatcctgtgtgcctgaagtcacaaaactaggtaatacgtggcatagctggaatctgaacctgaatttctaacatctgtacactccaaaccacctcgctaatgactgcgaactgtcctaggaattctcagaggccagaagttctgtcttgtttgctattttattttttgagacaggtctcattctgccattcaggctgaagtgtattggcacaatcatagctcactgcaaccttccaactcctgggaagtgtctgtacgtgtgtgtgtgtgtgtgtgtaagtgtctgtgtgtacgtgtaagtgtgtgtacgtgtgtgtgtgtgtgtgtgcgcacgcacgcgtgcccgttacgttgaccaggctggttgtaaactcctgggctcaagtgatccacctgcctcagcctccaaaagtgctgggattacatgtgtaagcctctgtgtccggcctctgtcttgttttctgccaagtcctcagggcctggatagggatcattgttggtttggtctgagttttttgtgttttaaaaaactgtggtaagatatacctaattgaagtttttattttttatggaggcataacatatctagaataaggtacacagatttttttttttttttttttgaggtggagttttgctcatgttgcccaggttggagtgcagtggcgcagtctcggctcactgcagcctccgcctcccaggttcaagcgattctcctgcctcagccttccaaagtgctgggaatacaggcgtgagccacctcgcctggcactcagatcttaatacatttttaggctgggcatggcggctgacacctataatctcagcactcaggaggccaaggagtgaggtttgtttgagcccaggagttcaagaccagcctaggcaacatagcaagaccctgtctctacaacaaatcttaaaaattagccgggtgtggtggcatgtgcctgctgtcccagctattcgggaggctgaggtgggaggatcgtttcaacctgggaggtcaaggctgctgtgagccgtgattgcatcgctgcactccagctcgggtgacagtgtgagactttgtctcaaacaacaaaacaaaacagttttccacatacacagcactattgtaaccactgcccaaatcaacttcattgatttttctttttttgagacagtcttcctctgtcacccaggctggagtgcaatggcatgatctcggctcactgcaacctccgcctcccgggttcaagagatttcctgcctcagcctcctgagtagctgggattacaggcatctgccaccacgcccagctactttttgtatttttagtagagatggggtttcaccatgttggtcaggctggtctcgaacccttgacctcaggtgatccatctgccttagcctcccaaagtgctgggattacaggtgtgagctactgtgcctggcctggtttttcaattgaggtgggggaatggggaaggctagagggcttccccacttccagagtagctgtgcgccttgtacttgctttattcactgatggtccttctaataatgcctgcggaaacccattttgtcaactgtggccacatgtaatctgtttgctctgtctgcag

SEQ ID NO: 30 - DNA, 2243 bp, Homo sapiens
gtttgtgcctcttgatggttgagttgctttcattaatctgtttgtattcagatagaagttttatctagagtaaggctgccaggttggtcattatattcaagctgttgaattaaaagcagaaagtagacttcctaagttgaaagagagcttaatgggttaggtattgcaaagataaacctagaaactggagtgaataactttctttaactttatttatttatttatttatttatttatttttgagatggagtttcgctcttctacccaggctggagggcagtggtgtgatgtcgcctcactgcaacctctgccttccggtttcaagcgattctcttgcctcagactcccgagtagctgggattacaggtgcctgccaccacgcctggctaatttttatatttttagcagagatggtgtttcaccatgttggccaggctggtctcgaactcctgacctcgcgatccgcccacctcaacctcctaaagtgctgggattacaggcgtgagccactgcatctggtcaactttctttttttgagactgagtctcactctagtggccaggctggagtgcaatggctcagtctcggcacactgccacctccacctcctgggttcaagcaattctcctgactcagcctcccgagtagctgggagcacaggtgtgctcacaggtgtgctcacctcacctggctgattttttgtatttttagtagagacggggcttcgccatgttggccaggctggtctcgaactcctgacctcaggtgatccacctgccttcgcctttcgaagtcctggggtttcaggcgggagccactgtgcccagccaagaataactttcatagttgctttgtaactatatttaaaaacaaaagagaaatggaaaactgttgatttgggtaccctgctcttgggtgctagggttgcttcagccataggatccacagctgagtctggacaatctgtgatgtggtgggtagaggactgtggtttgagtcagcatcttgactacatcaagtcagctcactgcttggcctcaatgcttgcaactataaaaagactgaggcccaaaagagatggtaaggatacaagtagtaaggttgtgaggctttaggcctcatgcaaaaatgtacaccctcagacctttgttccttttatctttctagccaagtaaaattatttataatctctacaaagagtggcaattaaaattatttataatcattaatttctactttatttatatacaaatactaatatatgtatatacatatacagaaacaggagaagctggtctgatactgtgaaaacatttcagcaggatcttggcatagtatatgtatatatacatatactaatatatgtttttctgtttgtaaattttttatttttttcctcatagtcgttaaagatgttatgtgaatgtatttttaattgtgtagtcaaacaataagataaaagttttactttaaaaaagttgccctttaggctgggcgtagtggctcacgcctataatcccagcacttgagaggctgaggtgggtggatcacctgaggtcagccattcgagaccagcctggccaacatagtgaaacaccatctctactaaaagcacaaaaattggcctggtatggtggcacacacctgtagtcccagctgcttaggcacgaggctgaggcacaaggatcacttgaacacaggaggcagaagttacagtgaaaaagaagttgccctttagttgttctcctgttggtggccatttaggtggattgtcacttttgctttcagagtcactgctgctgtaataatccttgtacatgtctgggtgtatgttgtgagtatttctctaggtagttatctagaagtgaattgctggttgatgatcatgtgcgttttattttaaatcaccactgaagaagtacgtactgacttatgctcccactctcattagcagttgctaagagtggaggctgttgaatgggccaggaacttgaggggttggtttggaccaaggtcatgggtgacagtagaaattggaagtagttggaatcctgcagtcttagaggcagagtgcgtaaaataatctgtagcatttgttatttattttcctgttgcttatttcaagtttcatgaagttggcatttctctccattgttcatgcaaaatttctcttacggtagattcaccataaacttttgttgatcattatctgaaatgattttctgaaacatttttttttctag

SEQ ID NO: 31 - DNA, 2182 bp, Homo sapiens
gtacggtagggccctgcgcgcacggcgccagagggatgggcgggtagagccaactgcctctggttctgctggcctccgctgctcgcgaagggattcctgctcccgggaggtgtaggagccgctttccagaagcacagcccagagacgtctgggcggcggcccacacaacgcatgtgttcggagctcgccgcgctctgcttttgctctaagcgggaaccatggcttctggccacgctggggaaccgaggaggtggccgcacccaagcaggggtcgaaagcccgggtggatgcggaacaaggatatgataggccttaagggtgggggatacctctgggctcgaaatcggcgggcggtgcaaaactcgaggtccagttctcggagcccatagagccaaaaaagcctcagcttgtccggggcgggttcttgaaagacggaaagcggctgagtaccacgcggcttgcatttttctcttgggacgctcgagaggtgggctccgtgagggcagctgctgcctgcagattatagggagccctttgcgcatttattaagaagctactggtgtatctcgggctgcgctaggcacggcgcatgcaaagatgaagcaggcagcatcccagcccttccgcacctcagacggtcagttgagtaggatccgccggtaccaactcctccttttaacaaatagggagaccgaaagctaggagacagtcagggatctctaagttcccagtgagtaggaggcagaggtgaggtgtagaactcgtttttgcatgtctctcgcctctagacgcacccttccctcatcccatgccctcccacctccgcccctacattaaaggtagcattggatcccggggccgttcagtgaagctagcaggtgtccgcaggaactcccttccccctgccaggctagaaaccttacaaggctgtctagaaatagcagtgatttgtaaggagagacccggctccagcttggtgactctgggctgactgcctgcctagaggtcctctcggatttttgccctttggagtggtgtcaaaactagacgtgatactttggggatgcagcctgtgatatttcctccagcgaatgcagtgcagggttggattaacaaggtggaaagaattcgagggttccaccaagtagctattaactctagggctgcaggcctcaggccttctgcagctatttctacactccctgtactgaaactatttcttcatactgggcctgacaggcctttgcaacaaggatcacggccgaagccacaccgtgcgcctccctcccggttggttaacaggccctggtttctagtattgcgatttaaagtctggcgctggctgcgcgccagacctgggaggctgccagctaggcttcacgttgctggcgtctgcttcggggcattcattaggtctgaagtctgaatcccagctccctccctctcacccactgagctgcatagctccagattgcctctgcttacgggcggggcttctcagccttctgccttctggcccgatgcccgcttcccaacggccggaggccgctagactaatcggcttcgccctgcgcgctgtaatgcgcatgcgcacgcgcacaagttcctgggcccgcccatcttccggacttgggcggggcgtaaaagccgggcgttcggaggacccagcaattagtctgatttccgcccacctttccgagcgggaaggagagccacaaagcgcgcatgcgcgcggatcaccgcaggctcctgtgccttgggcttgagctttgtggcagttaatggcttttctgcacgtatctctggtgtttacttgagaagcctggctgtgtccttgctgtaggagccggagtagctcagagtgatcttgtctgaggaaaggccagccccacttggggttaataaaccgcgatgggtgaaccctcaggaggctatacttacacccaaacgtcgatattccttttccacgctaaggtatgggccttcactcttcacagaccctgtcattaggcctttcaactctcttttggcaaccattaggttttttcccctccctttttagtcatctctagtgatttatagtggcaaatacccccaaaggaagtaaaatagcttaaaaaaatctcttggttaataaacattaaagaagctgtagtgacactaaatgtttttcctcctatag

SEQ ID NO: 32 - DNA, 4 bp, Adeno-associated virus
cagg

SEQ ID NO: 33 - DNA, 6 bp, Adeno-associated virus
cagctg

SEQ ID NO: 34 - DNA, 6 bp, Adeno-associated virus
caggtg

SEQ ID NO: 35 - protein, 738 aa, Adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 36 - protein, 601 aa, Adeno-associated virus
TAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 37 - protein, 535 aa, Adeno-associated virus
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 38 - DNA, 2217 bp, Adeno-associated virus
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 39 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 599 aa, Adeno-associated virus
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 41 - protein, 534 aa, Adeno-associated virus
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - DNA, 2211 bp, Adeno-associated virus
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 43 - DNA, 480 bp, Artificial Sequence
  Adeno-associate virus
atggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggc

SEQ ID NO: 44 - protein, 160 aa, Artificial Sequence
  Adeno-associate virus
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQG